(19) United States
(12) Patent Application Publication
(10) Pub. No.: US 2025/0327076 A1
(43) Pub. Date: Oct. 23, 2025

(54) CHEMICALLY MODIFIED ANTISENSE OLIGONUCLEOTIDES (ASOS) AND COMPOSITIONS FOR RNA EDITING

(71) Applicant: AIRNA Corporation, Cambridge, MA (US)

(72) Inventor: Tobias Merkle, Tübingen (DE)

(73) Assignee: AIRNA Corporation, Cambridge, MA (US)

(21) Appl. No.: 19/254,867

(22) Filed: Jun. 30, 2025

Related U.S. Application Data
(63) Continuation of application No. PCT/EP2024/073031, filed on Aug. 15, 2024.

(30) Foreign Application Priority Data
Aug. 15, 2023 (WO) PCT/EP2023/072474

(51) Int. Cl. C12N 15/113; A61P 11/00

(52) U.S. Cl. CPC C12N 15/113; A61P 11/00; C12N 2310/11; C12N 2310/315; C12N 2310/321; C12N 2310/322; C12N 2310/351; C12N 2320/32

(57) ABSTRACT

The invention relates to chemically modified oligonucleotides for use in site-directed A-to-I editing of a target RNA inside a cell with endogenous adenosine deaminase acting on RNA (ADAR), the oligonucleotide comprising a sequence capable of binding to a target sequence in a target RNA and a central base triplet (CBT) of 3 nucleotides (N₋₁N₀N₊₁), wherein N₀ is the central nucleotide directly opposite to a target adenosine in the target RNA that is to be edited, wherein the oligonucleotide comprises at least one internucleoside linkage that is a methanesulfonyl (mesyl) linkage.

### FIELD OF THE INVENTION

The present invention relates to the field of site-directed RNA editing, whereby an RNA sequence is targeted by an antisense oligonucleotide (ASO) for RNA editing of a genetic mutation (“compensatory editing”) or for editing of an RNA derived from a wildtype allele (“beneficial editing”).

### BACKGROUND OF THE INVENTION

RNA editing is a natural process through which some cells can make discrete changes to specific nucleotide seq within an RNA molecule in a site-specific way. Unlike DNA editing, the advantage of site-directed RNA editing is that it allows modification of the genetic information that leads to a modified protein in a more precise, efficient and safe manner. Contrary to DNA, RNA is generally quickly degraded and any errors introduced by off-target modifications to other RNAs will be washed out rather than permanently introduced into the modified DNA of a subject. RNA editing may also be less likely to cause an immune reaction since it is an editing mechanism naturally found in humans. Moreover, RNA editing might provide a more natural response than introducing an external, engineered gene.

Over the years, oligonucleotide therapeutics have been developed to silence, restore or modify the expression of disease-causing or disease-associated genes in, e.g., cancer and (other) genetic disorders. Such therapeutics include, e.g., antisense oligonucleotides (ASOs), small interfering RNA (SiRNA) and microRNA (miRNA) that interfere with coding and noncoding RNAs in a sequence specific manner. The relative ease and accuracy with which ASOs can be customized allows virtually any gene to be targeted. As a result, ASOs are the most clinically developed, with several drugs already approved by the U.S. Food and Drug Administration (FDA) and in clinical trials (Cideciyan et al., 2019; Gagliardi and Ashizawa, 2021).

Site-Directed RNA Editing (SDRE) describes the alteration of an RNA sequence by introducing or removing nucleotides from an RNA or by changing the character of a nucleobase by deamination. RNA editing enzymes are known in the art. The first RNA editing process discovered in mammals was the deamination of cytidine (C) by APOBEC proteins to form uridine (U) (Zinshteyn and Nishikura, 2009). To date, the two most useful and most studied types of RNA editing are cytidine (C) to uridine (U) (“C-to-U”) and adenosine (A) to inosine (I) (“A-to-I”) conversions. Notably, for therapeutic purposes in higher eukaryotes the most prevalent type of RNA editing is the “A-to-I” conversion, which is catalysed by the adenosine deaminases acting on RNA (ADARs) family.

Over the years, three vertebrate ADAR genes have been identified, which give rise to several ADAR proteins through alternative promoters or splicing (Wulff and Nishikura, 2010). ADAR proteins are expressed across various types of human tissues and can alter, inter alia, splicing and translation machineries, double-stranded RNA (dsRNA) structures as well as the binding affinity between RNA and RNA-binding proteins (Tomaselli et al., 2014; Zinshteyn and Nishikura, 2009). Of the three known ADAR genes, hADAR1 and KADAR2 are expressed in most tissues and encode active deaminases. Human ADAR3 (hADAR3) has been described to only be expressed in the central nervous system and reportedly has no deaminase activity in vitro. While all ADARs are multidomain proteins, comprising a targeting or dsRNA-binding domain (dsRBD) and a catalytic domain, ADAR1 proteins additionally comprise one or more Z binding domains, while splice variant ADAR2R and ADAR3 comprises an R domain (Zinshteyn and Nishikura, 2009; Wulff and Nishikura, 2010). Accordingly, the ADAR may be hADAR1, hADAR2 or hADAR3, or any variant thereof. The ability of ADARs to alter the sequence of RNAs has also been used to artificially target RNAs in vitro in cells for RNA editing

“A-to-I” editing was initially identified in Xenopus eggs (Bass and Weintraub, 1987; Rebagliati and Melton, 1987). Human cDNA encoding “double stranded RNA adenosine deaminase” was first cloned by Kim et al. (1994) and “A-to-I” conversion activity of the protein confirmed by recombinant expression in insect cells. Specifically, “A-to-I” editing changes the informational content of the RNA molecule, as inosine preferentially basepairs with cytidine and is therefore interpreted as guanosine (G) by the translational and splicing machinery. Therefore, ADARs have the effect of introducing a functional adenosine to guanosine mutation on the RNA level. Potentially, this approach may be used to repair genetic defects and alter genetic information at the RNA level.

ASOs are generally short (approx. 18 to 25 nucleobases in length) single-stranded synthetic RNA or DNA molecules, which use Watson-Crick base pairing to bind sequence specifically to the target RNA. They can be broadly classified into 1^{st} (Gen 1), 2^{nd} (Gen 2), and 3^{rd} (Gen 3) generation ASOs. Notably, ASO sequence and design are the primary drivers that determine the pharmacological and toxicological properties of the oligonucleotide.

Gen 1 ASOs were initially employed to inhibit translation of Rous sarcoma virus ribosomal RNA (Stephenson and Zamecnik, 1978). They are characterised in having a modified backbone, wherein the nucleotide linkages are modified by sulphur, methyl or amine groups to generate phosphorothioates (PS), methyl-phosphonates (MP), and phosphoramidates, respectively. Hence, ASOs can be chemically modified to improve their properties. For instance, ASOs can be modified to protect them against nucleases and to increase their effectiveness. While PS modifications seem to have a positive effect on ASOs stability and pharmacokinetics, the difference in chirality of PS linkages may have a substantial influence on the ASO's overall property (Iwamoto et al., 2017; Crooke et al., 2020).

Gen 2 ASOs show increased nuclease stability and affinity for their RNA targets, which has translated to improved potency and therapeutic index in the clinic. Gen 2 ASOs are typically modified using PS backbone modification and additionally carry alkyl modifications at the 2′ position of the ribose. Such 2′-sugar modifications may include 2′-O-methyl (2′-OMe), 2′-fluoro (2′-F), 2′-O-methoxyethyl (2′-MOE) modifications. Hence, these Gen 2 ASOs tend to be less toxic than PS-modified ASOs and have a slightly higher affinity for their target.

In comparison, Gen 3 ASOs tend to be even more heterogenous as they include a large number of chemical modifications that aim to further improve binding-affinity, stability, and pharmacokinetics (Quemener et al., 2019). Hence, the diversity of chemical modifications, together with the sequence of the ASO, offers considerable flexibility as relates to the therapeutic approach. That is, depending on their mechanism of action, ASOs can be used to degrade target mRNA, decrease protein levels, modify or correct splicing events, modulate RNA translation or target pathological coding or non-coding RNAs (Quemener et al., 2019).

ASOs can work through many mechanisms depending, in part, on the region in the RNA sequences that is targeted and ASO design/chemical properties. To ensure specificity, their sequences are generally complementary or at least partially complementary to the target RNA. However, in the case of site-directed mutagenesis, i.e., “A-to-I” RNA editing, the ASO targeting domain contains a mismatch opposite the targeted adenosine. It is to be noted that several endogenous substrates of ADAR contain mismatches and/or bulges (Thomas and Beal, 2017) and therefore could alter or even improve substrate recognition, if these features are mimicked in the ASO/resulting dsRNA.

Furthermore, ASOs can be chemically modified to improve their properties For instance, ASOs can be modified to protect them against nucleases and to increase their effectiveness. While phosphorothioate (PS) modifications seem to have a positive effect on ASOs stability and pharmacokinetics, the difference in chirality of PS linkages may have a substantial influence on the ASO's overall property. PS linkages can be found in two stereoisomers, Rp and Sp, and it is known from the art, that Rp and Sp linkages can influence properties such as, e.g., thermal stability, binding affinity, pharmacologic properties, etc., of the ASO. However, the benefit of Rp and Sp stereoisomers has been controversial (Iwamoto et al., 2017; Crooke et al., 2020).

The use of antisense oligonucleotides for site-directed RNA editing has previously been described (Vogel et al., 2014; Merkle et al., 2019) and ASO-based therapies have been gaining more and more traction over the past years for use in the treatment of different genetic disorders.

Loop-hairpin structured oligonucleotides have previously been described (WO 2020/001793) and have been used successfully to harness ADARs with chemically modified oligonucleotides. However, they are comparably large and-without being bound by any theory-the inventors believe that a more intelligent design of the ASO can form a substrate duplex that is also very well and quickly recognized by endogenous ADAR so that the large recruitment motifs can be omitted. For the delivery and manufacture this is a clear advantage as much shorter ASOs can be designed.

New designs for nucleoside analogues are constantly being investigated. These oligonucleotides typically are very rich in 2′-F-modifications within the 5′ half, which are generally present as blocks of 2′-F-modifications and uniform block of 2′-O-Methyl-modifications within the 3′ terminus on either side of the central base triplet (CBT), wherein the CBT has the general structure (5′-N₊₁N₀N₋₁-3′) and N₀ is the central nucleotide (N₀) directly opposite the target adenosine (A) to be edited, when the oligonucleotide is hybridized to the target RNA sequence. Further, some of these oligonucleotides contain almost complete stereopure PS-modified backbones and additional charge-neutral PN linkages (also stereopure), the latter of which is not yet applied in the clinics. That precise, site-specific RNA editing can be achieved by recruiting endogenous ADARs with antisense oligonucleotides has previously been shown by Merkle et al. (2019). They were able to demonstrate that chemically optimized ASOs can be used to recruit endogenous human ADARs to edit endogenous transcripts in a simple and programmable way with almost no off-target editing.

In WO 2020/001793, an artificial nucleic acid for site-directed “A-to-I” editing was provided, wherein the artificial nucleic acid comprised a targeting sequence and recruiting moiety. Similarly, WO 2018/041973 relates to ASOs that do not form an intramolecular hairpin or stem-loop structure. WO 2018/041973 specifically relates to chemically modified single-stranded RNA-editing oligonucleotides for the deamination of a target adenosine by an ADAR enzyme whereby the central base triplet (CBT) of three sequential nucleotides comprises a sugar modification and/or a base modification. It was found that deoxyribose at all three positions of the CBT is well tolerated and provides substantial stabilization against nuclease digestion.

Other prior art, such as WO 2021/071858, relates to oligonucleotides comprising a first and second domain, wherein the first domain comprises one or more 2′-F modifications and the second domain comprises one or more sugars that do not have a 2′-F modification, WO 2022/099159 relates to oligonucleotides with a first and second domain, wherein the domains comprise specific percentages of 2′-F modifications and aliphatic substitutions.

Research in the field of ASO optimisation for A-to-I editing has led not only to the identification of the CBT but also to a more thorough investigation of the region immediate 5′ and 3′ to the CBT. In addition to specifically looking at CBT modifications (e.g. 2′-F and 2′-FANA), WO 2021/243023 also mentions guide of targeting domain modifications 3′ to the nucleobase just outside the CET (at position +2 of an oligonucleotide comprising the structure [Am]-X¹-X²-X³-X⁴-[Bn], wherein X⁴ corresponds to the +2 position). It was found that editing the +2 position can affect the editing rate of the target. Improved editing was observed with a 2′-F modification at the +2 position.

However, despite being a promising technology, few ASOs have been marketed. This is due to difficulties pertaining to stability, cellular delivery, clinical efficacy, as well as off-target effects and/or preclinical toxicologic challenges. Hence, to translate ASO-based therapies into a widespread clinical success, it is crucial to overcome these different challenges. Accordingly, there is currently an unmet need for improved ASOs and effective therapies for the treatment of genetic disorders involving these improved ASOs. One aim of the invention is to provide ASOs with improved properties, including stability to aid in vivo delivery, and improved A-to-I editing.

### SUMMARY OF THE INVENTION

The inventors found that chemically modified antisense oligonucleotides comprising one or more mesyl phosphoramidate (or mesyl) linkages can be synthesised and used as alternatives to oligonucleotides comprising traditional internucleoside linkage modifications such as, e.g., phosphonothioate linkages (PS) and/or methylphosphonate (MP) linkages. It was observed that mesyl linkages have a beneficial effect and that by placing mesyl linkages at specific positions within the oligonucleotide, oligonucleotide stability and A-to-I target editing can be improved. For example, placement of mesyl linkages in the 5′ and 3′ flanking regions of the individual oligonucleotide enhanced editing. The inventors further identified key internal positions (e.g., position −2 and +13), where the mesyl linkage can be placed to improve oligonucleotide stability and activity.

The present invention provides oligonucleotides (or antisense oligonucleotides, ASOs) with desirable properties for in vitro and in vivo use. The problem solved by the instant invention lies in the provision of improved chemically modified ASOs capable of mediating a functional change from an adenosine (A) to a guanosine (G). Specifically, the invention relates to chemically modified oligonucleotides for use in site-directed A-to-I editing, comprising at least one linkage that is a methanesulfonyl (mesyl) linkage. To date, no prior art has been identified that teaches or suggests the oligonucleotides, compositions, and methods as provided herein, which are particularly effective in providing stable and less hydrophobic ASOs and compositions comprising the same for use in site-directed A-to-I editing of a target RNA.

The solution to the technical problem is achieved by the embodiments described herein and defined by the appended claims.

The present invention generally provides for chemically modified oligonucleotides for use in site-directed A-to-I editing of a target RNA inside a cell with endogenous adenosine deaminase acting on RNA (ADAR).

In a first aspect, the present invention provides a chemically modified oligonucleotide for use in site-directed A-to-I editing of a target RNA inside a cell with endogenous adenosine deaminase acting on RNA (ADAR), the oligonucleotide comprising a sequence capable of binding to a target sequence in a target RNA and a central base triplet (CBT) of 3 nucleotides (5′-N₊₁N₀N₋₁-3′), wherein N₀ is the central nucleotide directly opposite to a target adenosine in the target RNA that is to be edited, wherein the oligonucleotide comprises at least one linkage that is a methanesulfonyl (mesyl) linkage.

In a second aspect provided herein is a composition comprising the chemically modified oligonucleotide of the invention.

In a third aspect provided herein is a chemically modified oligonucleotide for therapeutic use.

In a fourth aspect provided herein is a chemically modified oligonucleotide of the invention or a composition of the invention for use in the treatment of a disease or disorder, where in the disease or disorder is selected form the group consisting of liver, metabolic, neurodegenerative, and/or cardiac or cardiovascular diseases associated with a gain-of-function (GOF) or loss-of-function (LOF) mutation

In a fifth aspect provided herein is a method for treating a subject suffering from a genetic disease or genetic disorder, comprising administering an effective amount of the chemically modified oligonucleotide of the invention or the composition of the invention to the subject.

In a sixth aspect provided herein is an in vitro method for site-directed A-to-I editing of a target RNA, the method comprising a step of contacting a target RNA with the chemically modified oligonucleotide of the invention or the composition of the invention.

The inventors found that chemically modified antisense oligonucleotides comprising one or more mesyl phosphoramidate (or mesyl) linkages can be synthesised and used as alternatives to oligonucleotides comprising traditional internucleoside linkage modifications such as, e.g., phosphonothioate linkage (PS), It was observed that by placing mesyl linkages at specific positions within the oligonucleotide A-to-I target editing could be improved. Specially, placement of mesyl linkages in the flanking regions of the individual oligonucleotide improved editing. The inventors further identified key internal positions in the ASO where the mesyl linkage should be placed to improve ASO activity.

### BRIEF DESCRIPTION OF DRAWINGS

The figures shown in the following are merely illustrative and shall describe the present invention in a further way. The figures shall not be construed to limit the present invention thereto.

FIG. 1 represents graphs showing (A) different types of internucleoside linkage modifications and positioning of individual PO, mesyl and PN internucleoside linkages within the oligonucleotide. (B) Mesyl Walk: respective editing (in %) depending on the specific positioning of the mesyl linkage at 4 nM and 20 nM.

FIG. 2 presents a bar graph showing the editing efficacy (in %) of different chemically modified oligonucleotides having a length of 30 nt to 38 nt and comprising a combination of different chemical modifications at the 2′-position of the sugar residue.

FIG. 3 presents a bar graph showing the editing (in %) of ASO candidates of varying asymmetries and lengths (30 nt, 34 nt, 36 nt, 38 nt) carrying different combinations of 2′F, PN, mesyl and 2′MOE modifications.

FIG. 4 presents a bar graph showing the editing (in %) of ASO candidates of varying asymmetries and lengths (30 nt, 34 nt, 36 nt, 38 nt) comprising different combinations of 2′F and 2′MOE modifications.

FIG. 5 presents a graph showing the editing (in %) of various GalNAc-conjugated SERPINA1 targeting oligonucleotides.

FIG. 6 presents a bar graph showing the editing (in %) of GalNAc-conjugated SERPINA1 targeting oligonucleotides in ASO transfected Piz mouse hepatocytes at concentrations 0.8 nM, 4 nM and 20 nM.

FIG. 7 shows (A) a layout of the in vivo study design and bar graphs showing (B) the editing (%) of the ABO candidates and (C) M-AAT (μM) levels.

FIG. 8 shows a bar graph depicting the results of a mesyl walk. The graph displays the editing (%) of various ASOs (“25-1-8” asymmetry) and a base mesyl-modified backbone (“+24, −2, −8”) and 1 additional moving mesyl linkage.

FIG. 9 shows bar graphs comparing the in vitro (A) and (B) and in vivo (C) and (D)) editing (in %) of ASO candidates comprising or 2 MP or 2 mesyl linkage modifications located at positions +24 and-8 (5′ and 3′ terminal position respectively) or 3 methylphosphonate (MP) or 3 mesyl linkage modifications located at positions +24, −2, and −8.

FIG. 10 shows (A) a layout of the in vivo study design and presents bar graphs (B) and (C) showing target editing (in %).

FIG. 11 shows the in vivo editing (in %) in liver tissue of ASO candidates comprising 2′MOE modifications or mesyl linkage or PN linkage modifications,

FIG. 12 presents a bar graph showing the in vivo editing (in %) of ASO candidates of different asymmetries comprising mesyl linkages in the 5′ and 3′ flanking regions.

FIG. 13 presents bar graphs showing (A) the editing (in %) of ASOs of various asymmetries, and (B) the editing (in %) of ASOs carrying an additional mesyl linkage modification at position +13.

FIG. 14 presents a bar graph showing the editing efficacy of surrogate ASO candidates comprising mesyl linkages in the 5′ and 3′ flanking regions.

FIG. 15 presents a bar graph showing the editing efficacy (in %) of various hACTB targeting oligonucleotides at 4 nM and 20 nM.

### DETAILED DESCRIPTION

### Terminology

In order that the present invention may be more readily understood, certain terms are first defined.

The articles “a” and “an” are used herein to refer to one or to more than one (I.e., to at least one) of the grammatical object of the article. By way of example. “an element” means one element or more than one element, e.g., a plurality of elements.

The terms “about” and “approximately” may be understood to permit standard variation as would be understood by those of ordinary skill in the art.

The term “Including” is used herein to mean, and is used interchangeably with, the phrase “including, but not limited to”. Likewise, the term “comprising” is used herein to mean, and is used interchangeably with, the phrase “comprising, but not limited to”.

As used herein, the expressions “mesyl phosphoramidate”, “methansulfonyl phosphoramidate” and “methanesulfonyl (mesyl)” internucleoside linkage (modification) have the same meaning and can be used interchangeably. The oligonucleotides of the invention contain at least one mesyl linkage, which means that at least one mesyl phosphoramidate linkage is incorporated into the ASO backbone instead of the natural phosphate (PO) linkage (i.e., phosphodiester group).

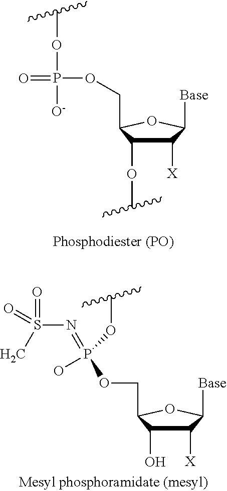

As used herein, a mesyl linkage at position +24 (N₊₂₄) indicates that the nucleotide at position 24 is linked via its phosphate to a (NSO₂CH₃) group and that the mesyl linkage is located between nucleotide 25 (N₊₂₅) and nucleotide 24 (Na₊₂₄). Likewise, if it is indicated the mesyl position is located at “−8” (N₋₈), this means that the mesyl linkage is between the nucleotides located at position −7 (N₋₇) and −8 (N₋₈). As used herein, nucleotide positions that are underlined indicate the terminal or penultimate positions containing a mesyl linkage modification, e.g. mesyl modification pattern “+24, −2, −8”.

As used herein the term “flanking region” refers to the 5′ and/or 3′ region on the oligonucleotide is adjacent or directly adjacent to the N₀ on the 5′ and/or 3 portion of the oligonucleotide. In one embodiment, the flanking region is located directly adjacent to N₀. Alternatively, in one embodiment, a flanking region is located anywhere upstream and/or anywhere downstream of N₀. In one embodiment, the flanking region is located at the far end of the 5′ terminus and/or at the far end of the 3′ terminus. The flanking region may comprise one or more nucleotide, i.e., a range of nucleotides. For instance, the flanking region my comprise 1, 2, 3, 4, 5, 6, 7, 8, 9 or more nucleotides 5′ and/or 3′ to N₀. That is, in some embodiments, the flanking region comprises the entire region 5′ and/or 3′ to N₀. In other embodiments the flanking region comprises the outermost 1, 2, or 3 nucleotides at the 5′ and/or 3′ terminus.

As used herein, the term “nucleic acid” is intended to include any DNA molecules (e.g., cDNA or genomic DNA) and any RNA molecules (e.g. mRNA) and analogues of the DNA or RNA generated using nucleotide analogues. Oligonucleotides can be single-stranded (ss) or double-stranded (ds). A single-stranded oligonucleotide can have double-stranded regions (formed by portions of the single-stranded oligonucleotide). A double-stranded oligonucleotide can have single-stranded regions, for example, at regions where the two oligonucleotide chains are not complementary to each other. Each component of the DNA or RNA can be modified and categorized by modification of (1) the internucleoside linkage, (2) the deoxyribose/ribose, and/or (3) the nucleobase.

The term “nucleobase” or “base” refers to biological building blocks that can form nucleosides, which, in tum, may be components of nucleotides. Naturally occurring bases are generally guanine, (B), adenine, (A), cytosine, (C), thymine, (T), and uracil (U), which are derivatives of purine or pyrimidine. Cytosine, thymine, and uracil are pyrimidine bases that are generally linked to the backbone through their 1-nitrogen. Adenine and guanine are purine bases and generally linked to the backbone through their 9-nitrogen. It should be understood that naturally and non-naturally occurring base analogues are also included and that the term “nucleobase” also includes “modified nucleobases”.

Within the context of this invention, the term “modified nucleobase” and “modified base” may be used interchangeably with the term “nucleobase” A nucleobase may be a nucleobase, which comprises a modification. In some embodiments, a modified nucleobase is capable of at least one function of a nucleobase, e.g., forming a moiety in a polymer capable of base-pairing to a nucleic acid comprising an at least complementary sequence of bases. In one embodiment, the modified nucleobase is capable of increasing hydrogen bonding, base pair stacking interactions and/or stabilizing a nucleic acid complex. The modified nucleobase (e.g., Benner's base) may be capable of mimicking the N3 protonated cytosine base. In some embodiments, a modified nucleobase is substituted A, T, C, G, or U, or a substituted tautomer of A, T, C, G, or U. In some embodiments, a modified nucleobase in the context of oligonucleotides refer to a nucleobase that is not A, T, C, G or U. Modifications include but are not limited to nonstandard nucleobases 5-methyl-2′-deoxycytidine (m⁵C), pseudouridine (DU), dihydrouridine, inosine (I), and 7-methylguanosine. In some embodiments, the modification is iso-uridine (SbU). Other modifications may include nucleobase replacement by (N) heterocycles (e.g., nebularine) or aromatic rings that stack well in the RNA duplex, such as, e.g., a Benner's base Z (and/or analogues) or 8-oxo-adenosine (B-oxo-A). As used herein, the term “Benner's base Z” refers to the pyrimidine analogue 6-amino-5-nitro-3-(1′,β-D-2′-deoxyribofuranosyl)-2(1H)-pyridone (dZ). In one embodiment, a modification includes the introduction of nucleobase analogues or simple heterocycles that boost editing. As used herein, and as commonly understood by the skilled person in the art, the expression “derivative thereof” refers to a derivative of a (modified) nucleobase, nucleoside of nucleotide. For example, a derivative may be a corresponding nucleobase, nucleoside or nucleotide that has been chemically derived from said nucleobase, nucleoside or nucleotide. For instance, a derivative of deoxycytidine may include fluoro-modified deoxycytidine, S-methyl-2′-deoxycytidine (m⁵C), or ribocytidine.

The term “nucleoside(s)” refers to a moiety wherein a nucleobase or a modified nucleobase is covalently bound to a sugar or a modified sugar. In some embodiments, a “nucleoside” refers to a nucleoside unit in an oligonucleotide or a nucleic acid. The term “nucleoside(s)” encompasses all modified versions and derivatives “modified nucleobases”.

The term “nucleotide(s)” as used herein refers to a monomeric unit of a polynucleotide that consists of a nucleobase, a sugar, and one or more linkages (e.g., phosphate linkages in natural DNA and RNA). In some cases, the linkage may be a non-naturally occurring and/or modified linkage. In some embodiments, the linkage may be an internucleoside linkage as described herein. In one specific embodiment, the modified linkage is a PS linkage. In some embodiments, a “nucleotide” refers to a nucleotide unit in an oligonucleotide or a nucleic acid. The term “nucleotide(s)” encompasses all modified versions and derivatives of “nucleosides” and “modified nucleobases”.

The term “oligonucleotide(s)” as used herein is defined as is generally understood by the skilled person as a molecule including two or more covalently linked nucleosides. They can comprise DNA and/or RNA. The oligonucleotides may have a backbone comprising deoxyribonucleotides and/or ribonucleotides.

The term “internucleoside linkage” refers to a linkage between adjacent nucleosides. “Internucleoside linkage” and “linkage” may be used interchangeably. Linkages may be continuous (consecutive) or discontinuous (interrupted). As used herein, the term “discontinuous” or “interrupted” means that there are not more than, e.g., 4, 5, 6, 7 or more consecutive internucleoside linkage modifications of the same modification. In some embodiments, the naturally occurring PO linkages are replaced by modified internucleoside linkages. Hence, in some embodiments, the linkage is a non-natural internucleoside linkage.

As used herein the term “stereopure” or “stereorandom” refers to chemically modified oligonucleotides. Specifically, the term “stereopure” refers to oligonucleotides that are chirally pure (or “stereochemically pure”). The term “stereorandom” refers to racemic (or “stereorandom”, “non-chirally controlled”) oligonucleotides. Hence, the oligonucleotides of the invention comprise 1, 2, 3, 4, 5, 6, 7, 8, 9, 10 or more stereorandom internucleoside linkages (mixture of Rp and Sp linkage phosphorus at the internucleoside linkage, e.g., from traditional non-chirally controlled oligonucleotide synthesis). In one embodiment, an internucleoside linkage is a phosphorothioate (PS) linkage. In one embodiment, an internucleoside linkage is a stereorandom PS linkage. In one embodiment, an internucleoside linkage is a chirally controlled PS linkage, In one embodiment, an internucleoside linkage is not chirally controlled. In one embodiment, an internucleoside linkage is not a chirally controlled PS linkage.

As used herein the term “antisense oligonucleotide” or “ASO” refers to a strand of nucleotide analogue that hybridizes with the complementary (target) RNA in a sequence-specific manner via Watson-Crick base pairing. The ASO may be chemically modified. The terms “antisense oligonucleotide” and “oligonucleotide” may be used interchangeably.

As used herein, the term “target RNA” refers to an RNA, which is subject to the editing process, and “targeted” by the respective ASOs of the invention.

As used herein, the term “off-target” or “off-targeting” refers to non-specific and/or unintended genetic modification(s) of the target. Off-target editing may include unintended point mutations, deletions, insertions, inversions, and translocations. For instance, off-target editing may arise from the promiscuous reactivity of the deaminase enzymes.

The term “modified sugar” refers to a moiety that can replace a naturally occurring sugar. A modified sugar may mimic the spatial arrangement, electronic properties, or some other physicochemical property of a sugar. The naturally occurring sugar is generally the pentose deoxyribose or ribose, though it should be understood that naturally and non-naturally occurring sugar analogues are also included. For example, sugars may comprise C4 sugars, C5 sugars and/or C6 sugars. In some embodiments, a modified sugar is substituted. In some embodiments, a modified sugar is a sugar that is not ribose or deoxyribose as typically found in natural RNA or DNA (e.g., arabinose). In some embodiments, a modified sugar comprises a 2′-modification. Examples of useful 2′-sugar modifications include. e.g., 2′-ribose (RNA), 2′-deoxyribose (DNA), 2′-arabinose etc. Those skilled in the art, will appreciate that various types of 2′-sugar modifications are known that can be used in accordance with the present disclosure. In one embodiment, the 2′-sugar modification is 2′-ribose. In one embodiment, the 2′-sugar modification is 2′-deoxyribose. The term “locked nucleic acid” (LNA) or “locked nucleic acids” (LNAs) are also known as bridged nucleic acid (BNA) and refers to modified RNA nucleotide in which the ribose moiety is modified with an extra bridge connecting the 2′ oxygen and 4′ carbon. In some embodiments, a modified sugar is a bicyclic sugar, e.g., a sugar used in locked nucleic acid (LNA), BNA, etc. In some embodiments, a modified sugar is an LNA sugar. In some embodiments, a modified sugar is an BNA sugar. In some embodiments, a sugar modification is 2′-OMe, 2′-O-methoxyethyl (2′-MOE), 2′-F. 5′-vinyl, or S-constrained ethyl (S-cEt). In one embodiment, a 2′-modification is a C2-stereoisomer of 2′-F-ribose. In one embodiment, a 2′-modification is 2′-F. In one embodiment, a 2′-modification is 2′-FANA. In one embodiment, a modified sugar is a sugar of morpholino. In one embodiment, the oligonucleotide comprises, e.g., an UNA (unlocked nucleic acid), a PMO (phosphorodiamidate linked morpholino) or a PNA (peptide nucleic acid). In one embodiment, the nucleic acid analogue is a PNA (peptide nucleic acid). In one embodiment, the nucleic acid analogue is PMO (phosphorodiamidate linked morpholino).

The term “FANA” or “FANA-modified” refers to 2′-fluoroarabinoside modified nucleobases and/or oligonucleotides comprising such nucleobases. For example, the expression “FANA-cytidine” refers to a cytidine that comprises a 2′-fluoro-beta-D-arabinonucleic acid sugar modification. Within the context of this invention, the expression “a derivate thereof” refers to a corresponding nucleotide(s) or oligonucleotide(s) that has been chemically derived from said nucleotide or oligonucleotide(s).

As used herein, the term “complementary”, “partially complementary” or “substantially complementary” refer to nucleic acid sequences, which, due to their complementary nucleotides, are capable of specific intermolecular base-pairing. The oligonucleotide may comprise a nucleic acid sequence complementary to a target sequence, e.g., SERPINA1, or any other target sequence. The ASO may be self-complementary. The ASO may be complementary to a coding or non-coding sequence. As those skilled in the art appreciate, perfect (e.g., 100%) complementarity or pairing is not required and one or more wobbles (wobble base pairing), bulges, mismatches, etc. may be tolerated. The one or more wobbles, bulges, mismatches, etc. may be within or outside the CBT. Hence, in one embodiment, the ASOs comprise a wobble base outside the CBT. In one embodiment, the ASO comprises a mismatch outside the CBT. For example, the ASOs may include a mismatch opposite the target adenosine. Hence, the complementarity of the ASOs may be 100%, except at the nucleoside opposite to a target nucleoside to be edited. In one embodiment, complementarity is at least 80%, 85%, 90%, 95% In one embodiment complementarity is 85%-898%, In one embodiment, the ASO comprises 1, 2, 3, 4, 5 or more mismatches when aligned with the target nucleic acid. In one embodiment, one or more mismatches are independently a wobble base paring. In one embodiment, the ASOs comprise up to 4 mismatches or wobble bases outside the CBT. In one embodiment, the ASOs comprise up to 3 mismatches or wobble bases outside the CBT.

The term “mutation” as used herein, refers to a substitution of a residue with another residue within a sequence, a.g., a nucleic acid sequence or amino acid sequence, or to a deletion or insertion of one or more residues within a sequence, e.g., point mutation. Mutations are typically described herein by identifying the original residue followed by the position of the residue within the sequence and by the identity of the newly substituted residue. Notably, the invention is not limited to correcting mutations, as it may instead be useful to change a wildtype sequence into a mutated sequence using the ASOs of the invention. Various methods for making amino acid substitutions are well known in the art and are provided by, e.g., Green and Sambrook, Molecular Cloning: A Laboratory Manual (4th ed., Cold Spring Harbor Laboratory Press. Cold Spring Harbor. N.Y. (2012).

As used herein, the term “beneficial editing” refers to the editing of a target sequence (or base) derived from a wildtype allele (not a mutated allele) in order to. e.g. modulate the function of a wildtype protein in a useful way to prevent or treat a disease. For example, beneficial editing may include sites, such as STAT1 Y701, NLRP3 Y166 and CTNNB1 T41 that are not causes for genetic diseases but rather represent wildtype protein sites. These sites may be changed (no underlying G-to-A mutation) to alter the function of the wildtype protein.

The term “compensatory editing” refers to the modification of RNA nucleotides to change and correct one or more detrimental or unfavourable changes in the RNA sequence when compared to wildtype, e.g., a compensatory A-to-I change could help to functionally compensate for an otherwise non-editable mutation to ameliorate a disease phenotype.

The term “adenosine deaminase(s)” or “adenosine deaminase(s) acting on RNA”[ADAR(S)], as used herein, refers to any (poly)peptide, protein or protein domain or fragment thereof capable of catalysing the hydrolytic deamination of adenosine to inosine. The term thus not only refers to full-length and wild type ADARs but also to a functional fragment or a functional variant of an ADAR. In some embodiments, the ADAR is an (endogenous) adenosine deaminase catalysing the deamination of adenosine to inosine or deoxy-adenosine to deoxyinosine. In some embodiments, the ADAR catalyses the deamination of adenine or adenosine in deoxyribonucleic acid (DNA) or in ribonucleic acid (RNA). The ADAR may be a human ADAR. The ADAR may be an endogenous ADAR. Accordingly, in some embodiments, the ADAR is an endogenous human ADAR1, ADAR2 or ADAR3 (hADAR1, hADAR2 or hADAR3), or any fragment or isoform(s) thereof (e.g., hADAR1 p110 and p150).

The term “guide RNA” (gRNA) or “guide oligonucleotide” refers to a piece of RNA or oligonucleotide (comprising RNA and/or DNA) that functions as a guide for enzymes, with which it forms complexes. The guide RNA or guide oligonucleotide may comprise endogenous and/or exogenous sequences. Guide RNAs bind to their target in a sequence-specific manner. Guides can be used in vitro and in vivo. For example, the guide RNA or guide oligonucleotide directs the base-modifying activity/editing function (e.g., ADAR) to the target to be edited in trans.

As used herein, the terms “disease” or “disorder” are used interchangeably to refer to a condition in a subject. In certain embodiments, the condition is a disease in a subject, the severity of which is decreased by inducing an immune response in the subject through the administration of a pharmaceutical composition.

As used herein, the term “effective amount” in the context of administering a therapy to a subject refers to the amount of a therapy which has a prophylactic and/or therapeutic effect(s).

As used herein, the term “in combination” in the context of the administration of two or more therapies to a subject, refers to the use of more than one therapy (e.g., more than one prophylactic agent and/or therapeutic agent). The use of the term “in combination” does not restrict the order in which therapies are administered to a subject. For instance, one or more ASOs may be used in combination.

As used herein, the terms “prevent”, “preventing” and “prevention” refer to the inhibition of the development or onset of a disease or symptoms thereof. In one embodiment, it relates to the administration of the compound to a patient who is known to have an increased risk of developing a certain condition, disorder, or disease.

As used herein, the terms “treat”, “treatment”, and “treating” refer to the halting, ceasing the progression of, or (partially) reversing particular symptoms of a disease or disorder. Beneficial or desired clinical results include, but are not limited to, alleviation of symptoms; diminishment of the extent of a condition, disorder, or disease stabilized (i.e., not worsening) state of condition, disorder, or disease; delay in onset or slowing of condition, disorder, or disease progression; amelioration of the condition, disorder, or disease state or remission (whether partial or total), whether detectable or undetectable an amelioration of at least one measurable physical parameter, not necessarily discernible by the patient; or enhancement of improvement of condition, disorder, or disease. Treatment includes eliciting a clinically significant response without excessive levels of side effects. Treatment also includes prolonging survival as compared to expected survival if not receiving treatment.

The terms “subject” or “patient” are used interchangeable and relate to an animal (e.g., mammals) that may need administration of the compound of the invention in the field of human or veterinary medicine. In specific embodiments. the subject is a human. The subject may be administered the oligonucleotide of the invention for beneficial editing. The subject may be administered the oligonucleotide of the invention for compensatory editing.

As used herein, the term “pharmaceutically acceptable” means approved by a regulatory agency. The term “carrier” refers to a diluent, adjuvant, excipient, or vehicle with which the pharmaceutical composition is administered. Saline solutions and aqueous dextrose and glycerol solutions can also be employed as liquid carriers. particularly for injectable solutions. Suitable excipients include starch, glucose, lactose, sucrose, gelatine, malt, rice, flour, chalk, silica gel, sodium stearate, glycerol monostearate, talc, sodium chloride, dried skim milk, glycerol, propylene, glycol. water, ethanol and the like. The formulation should suit the mode of administration.

Unless otherwise defined, all technical and scientific terms used herein have the same meaning as commonly understood by one of ordinary skill in the art to which this invention belongs. Methods and materials are described herein for use in the present disclosure; other, suitable methods and materials known in the art can also be used.

### Oligonucleotides

Described inter alia, chemically modified antisense oligonucleotides (ASOs). While not intending to be bound by any particular theory of operation, it is believed that nucleobase and backbone linkage modifications are useful in stabilising ASOs, improving their editing efficacy, reducing their off-target editing and/or hydrophobicity. Since the one or more modifications can be synthetically transferred to various oligonucleotide sequences, such modifications have the potential to improve the editing efficacy of oligonucleotides with different target specificities. The ASOs of the invention can be used for several purposes associated with “A-to-I” conversions. That is, the ASOs of the invention are not just limited to correcting G-to-A mutations but are also useful in changing a wildtype sequence into a mutated sequence in order to modulate protein expression and/or function (“beneficial editing”). Thus, the oligonucleotides may be used as active agents to treat genetic disorders or diseases associated with one or more G-to-A mutations or to change wildtype sequences.

While the oligonucleotides of the invention comprise different types of internucleoside linkages, the inventors have shown oligonucleotides comprising at least one linkage that is a methanesulfonyl (mesyl) linkage have enhanced RNA editing. That is, the inventors h t the oligonucleotides of the invention do not require all of the internucleoside linkages to carry a mesyl linkage, provided that a minimum level of internucleoside modification is incorporated. Accordingly, oligonucleotides of the invention comprise at least one methanesulfonyl (mesyl) linkage modification,

The inventors have also realised that to provide shorter oligonucleotides for RNA editing, and to achieve a beneficial balance of high editing efficacy and low hydrophobicity, it is desirable to incorporate certain backbone linkage and nucleobase modifications and/or mixtures thereof into the oligonucleotides. In particular, depending on the length of the ASO, it is desirable that the ASOs have a mixture of different modifications at the 2′-position of the sugar residue. The inventors have specifically realised that introducing mesyl modifications into the core oligonucleotide backbone reduces overall hydrophobicity of the ASO as well as immune activation Hence, according to the invention, the oligonucleotide comprises at least one internucleoside linkage that is a methanesulfonyl (mesyl) linkage.

Provided herein is a chemically modified oligonucleotide for use in site. directed A-to-I editing of a target RNA inside a cell with endogenous adenosine deaminase acting on RNA (ADAR), the oligonucleotide comprising a sequence capable of binding to a target sequence in a target RNA and a central base triplet (CBT) of 3 nucleotides (5′-N₊₁N₀N₋₁-3′), wherein N₀ is the central nucleotide directly opposite to a target adenosine in the target RNA that is to be edited, and wherein the oligonucleotide comprises at least one linkage that is a methanesulfonyl (mesyl) linkage.

The oligonucleotides of the invention benefit from having a base level of internucleoside linkage modifications, i.e., at least one linkage that is a methanesulfonyl (mesyl) linkage. This will have a positive effect on, inter alia, the pharmacokinetics as well as stability, protein binding, intracellular localization. hydrophobicity and cytotoxicity of ASOs. The oligonucleotide of the invention may in addition to the mesyl linkage(s) comprise further internucleoside linkage modifications.

The chemically modified oligonucleotides of the invention comprise at least one linkage that is a methanesulfonyl (mesyl) linkage. In one embodiment, the mesyl linkage content is at least 10% or 15%, that is at least 10% or 15% of the internucleoside linkages are methanesulfonyl (mesyl) linkages. In one embodiment, the mesyl linkage content is at least 20%, 25%, 308%, 353%, 40%, 46%, 50%, 55%, 60%, 65%, 70%, 80%, or 90%. In some embodiments, at least 10% of linkages are mesyl modified internucleoside linkages, optionally at least 20%, 30%. 40% or 503%. In one embodiment, no more than 95%. 90%, 86%, 80%, 70%, 60%, 50%, 40%, or 30% of the linkages are mesyl linkages. In one embodiment, at least 5% of the internucleoside linkages are methanesulfonyl (mesyl) linkages. In one embodiment, at least 8% of the internucleoside linkages are methanesulfonyl (mesyl) linkages.

In one embodiment, the mesyl linkage content is 15-90%, 15-80%. 15-70%. 15-60%, 20-90%, 10-80%, 20-80%, 25-80%, 30-80%, 30-90%, 40-90%, 40-80%, 40-70%, 45-90%, 45-85%, 45-75%, 45-70%, 45-60% or 45-55%. In one embodiment, 15-90% of the linkages are mesyl linkages. In one embodiment. 40-80% of the linkages are mesyl linkages. In one embodiment, 45-60% of the linkages mesyl linkages. In one embodiment, the mesyl linkages content is 20%, 30%, 40%, 45%, 50%, 60%, 70%, 80%, or 90%. In one embodiment, the mesyl linkages content is 30%. In one embodiment, the mesyl linkages content is 15%.

In one embodiment, no more than 95%, 90%, 85%, 80%, 70%, 60%, 50%, 40%, 30% or 20% of the linkages outside the CBT are mesyl linkages, or 15-90% of the linkages are mesyl linkages, preferably wherein 40-80%, most preferably 45-60%. of the linkages are mesyl linkages. In one embodiment: 15-90% of the linkages are mesyl linkages, preferably wherein 40-80%, most preferably 45-60%, of the linkages are mesyl linkages. In one embodiment, the mesyl linkages content is at least 15%, 20%, 30%, 40%, 50%, 60%, 70%, 80% or 80%. In one embodiment, the mesyl linkages content is no more than 95%, 90%, 85%, 80%, 75%, 70%, 65%, 60%, 55%, 50%, 45%, 40%, 35%, 30%, 25% or 20%. In one embodiment, 100% of internucleoside linkages are mesylated. In one embodiment, the oligonucleotide is fully mesylated, i.e., all backbone linkages are mesyl linkages.

In one embodiment, the between 2 and 33 mesyl linkages are methanesulfonyl (mesyl) linkages. In one embodiment, the between 2 and 30, between 5 and 25, between 50 and 20, or between 2 and 20 mesyl linkages are methanesulfonyl (mesyl) linkages. In one embodiment, the oligonucleotide comprises no more than 8, 7, 6, 5, 4, or 3 mesyl linkages. In one embodiment, a) at least 5%, 8%, 10%, 20%, 30%, 40%, 50%, 60%, 70%, 80%, or 90% the internucleoside linkages are methanesulfonyl (mesyl) linkages; b) between 2 and 20 mesyl linkages are methanesulfonyl (mesyl) linkages, preferably between 2 to 8; or c) the chemically modified oligonucleotide is fully mesylated. In one embodiment, the oligonucleotide contains 4 mesyl linkages (e.g., AI-3059). In one embodiment, the oligonucleotide contains 5 mesyl linkages (e.g., AI-3008, AI-2693). In one embodiment, the oligonucleotide contains 6 mesyl linkages (e.g., AI-1068, AI-1686, AI-1701). In one embodiment, the oligonucleotide contains 8 mesyl linkages (e.g., AI-1691). The specificity sequence of the ASOs of the invention may be described as a 5′ to 3′ (antisense) oligonucleotide or polynucleotide sequence. The specificity sequence and target region will be described with reference to the target “A” (adenosine to be edited). The target A is located at the “zero position” within the target sequence. The specificity sequence site within the ASO that is directly opposite the target “A” to be edited is referred to as the zero position (N₀). The downstream positions (i.e., 3′ to the N₀ position) are marked −1, −2, −3, etc (N₋₁, N₋₂, N⁻³, etc.), while the upstream (i.e., 5′ to the N₀ position) positions are numbered +1, +2, +3 (N₊₁, N₊₂, N₊₃, etc.). Accordingly, an oligonucleotide of the invention may have a general sequence of 5′- . . . N₊₅^{a}N₊₄^{b}N_{+c}^{c}N₊₂^{d}N₊₁^{e}N₀^{f}N₋₁^{g}N₋₂^{h}N₋₃ⁱN₋₄^{j} N₋₅, . . . , −3′.

Mesyl linkages may be located at any nucleotide position within the oligonucleotides of the invention. For instance, one or more mesyl linkage modifications may be located at internal positions anywhere along the entire length of the oligonucleotide or (only) at the 5′ and/or 3′ terminal ends of the oligonucleotide. Alternatively, in one embodiment, the mesyl linkage is located within a 5′ and/or a 3′ terminus flanking region(s) outside of the CBT (5-N⁺¹N₀N⁻¹-3), i.e., upstream of N₊₁ and/or downstream of N₋₁. In one embodiment, the mesyl linkage is located within the CBT, i.e., between position +1 and 0 and/or between positions 0 and −1. In one embodiment, the mesyl linkage is directly (i.e., adjacent to) upstream of N⁺¹ (at position +2). In one embodiment, the mesyl linkage is directly downstream (i.e., adjacent to) of N₋₁ (at position −2). In one embodiment, the oligonucleotide comprises a mesyl linkage within the flanking region 3′ to N₀. In one embodiment, the oligonucleotide comprises a mesyl linkage within the flanking region 5′ to N₀. In one embodiment, the oligonucleotide comprises a mesyl linkage within each of the 6′ and 3′ flanking regions. In one embodiment, the oligonucleotide comprises 1, 2, 3, 4, 5, 6, 7, or 8 mesyl linkages within the flanking regions 3′ and/or 5′ to N₀. In one embodiment, the oligonucleotide comprises between 1-20 mesyl linkages 5′ to N₀. In one embodiment, the oligonucleotide comprises between 1-10 mesyl linkages 3′ to N₀.

In one embodiment, the mesyl linkage is located within the 3′ and/or 5′ flanking region(s) outside of the CBT. Since oligonucleotides may vary in overall length, the length of the 3′ and/or 5′ terminal flanking regions of each oligonucleotide may vary in length accordingly. In one embodiment, the flanking regions at the 5′ and 3′ termini have the same length. In one embodiment, the flanking regions at the 6′ and 3′ termini have different lengths.

In one embodiment, the oligonucleotide comprises 2, 3, 4, 5, 6 or 7 mesyl modifications within a 3′ and/or 5′ flanking region(s) outside of the CBT. In one embodiment, the oligonucleotide comprises at least 2, 3, 4, 5, 6, or 7 mesyl modifications within a 3′ and/or 6′ flanking region(s) outside of the CBT. In one embodiment, the 5′ terminus flanking region comprises the terminal 10, 9, 8, 7, 8, 5, 4, 3, 2 or 1 nucleotide (s) of the oligonucleotide, preferably wherein the 6′ terminus flanking region comprises the outermost 6, 5, 4, 3, 2, or 1 nucleotide(s), In one embodiment, the 3′ terminus flanking region comprises the terminal 7, 6, 5, 4, 3, 2 or 1 nucleotide(s) of the oligonucleotide, preferably wherein the 3′ terminus flanking region comprises the outermost 4, 3, 2, or 1 nucleotide(s).

In one embodiment, the oligonucleotide comprises 1, 2, or 3 mesyl linkages within the 3′ and/or 5′ terminus flanking region(s). In one embodiment, the 1, 2, or 3 mesyl linkages within the 3′ and/or 5′ terminus flanking region(s) are between the terminal 1, 2, 3 and 4 nucleotides of the 5′ and/or 3′ terminus. That is, in one embodiment, the 1, 2, or 3 mesyl linkages are located between the outermost 5 nucleotides of the 5′ and/or outermost 4 nucleotides of the 3′ terminus of the oligonucleotide. In one embodiment, the 1 or 2 mesyl linkages are located between the outermost 2 or 3 nucleotides of the 5′ and/or outermost 2 or 3 nucleotides of the 3′ terminus of the oligonucleotide.

In other embodiments, the oligonucleotide comprises a mesyl linkage between the terminal and penultimate nucleotide of the 5′ terminus and a mesyl linkage between the terminal and penultimate nucleotide of the 3′ terminus In other embodiments, the oligonucleotide comprises 2 mesyl linkages at the 6′ terminus. which are placed between the terminal 3 nucleotides of the 5′ terminus. In other embodiments, the oligonucleotide comprises 2 mesyl linkages at the 3′ terminus, which are placed between the terminal 3 nucleotides of the 3′ terminus. In one embodiment, the oligonucleotide comprises 2 mesyl linkages at the 5′ terminus, which are placed between the terminal 3 nucleotides of the 5′ terminus and 2 mesyl linkages at the 3′ terminus, which are placed between the terminal 3 nucleotides of the 3′ terminus.

In one embodiment, a mesyl linkage is located between any two of the nucleotide positions of the oligonucleotide. That is, in an oligonucleotide with a length of 34 nt, a mesyl linkage may be located at any of the 34 positions of the oligonucleotide (e.g., at position 5′-+24, +23, +22, [ . . . ], . . . 0, . . . [ . . . ], −3, −4, −5, −6, −7, −8 -3′). In one embodiment, a mesyl linkage is located between the outermost 1-5, 1-6, 1-7, 1-8, 1-9 or 1-10 nucleotides. In one embodiment, the mesyl linkage is located at one or more of positions+positions +28, +27, +26, +25, +24, +23, +22, +21, +20, +19, +14, +13, +12, +11, +10, +5, +4, +3, −2, −5, −7, −8, −9, −11, −12, −13, −14, −15, −16, −17, −18, and/or −19. In one embodiment, the mesyl linkage is positioned at one or more of the following positions +27, +26, +25, +24, +23, +22, +21, +20, +19, +13, +12, +11, +8, +5, +4, −2, −6, 7, and −8. In one embodiment, the mesyl linkage is located at one or more of the following positions selected from: +24, +23, +21, +13, +4, −2, −6, −7, and −8.

In some embodiments, there is no mesyl linkage modification at one or more of the following positions: +19, +18, +17, +16, +16, +14, +10, +9, +8, +7, +6, +3, +2, +1, 0, −1, −2, −3, −4, −5, −6, −7, −8, −9, and −10. In one embodiment, there is no mesyl linkage modification at one or more of the following positions: +18, +17, +16, +15, +9, +8, +7, +6, +2, +1, 0, −1, −3, −4, −6, −7, −8, and −10. In one embodiment, there is no mesyl linkage modification at one or more of the following positions: +18, +17, +15, +8, +5, +1, −3, and −6.

The inventors have found that oligonucleotides comprising mesyl linkages show improved editing compared to those oligonucleotides that do not (e.g., Example 5). The oligonucleotides of the invention may thus contain internal mesyl linkages or mesyl linkages at the 5′ and/or 3′ terminal ends. As used herein, “internal mesyl linkages” are those linkages that are not located between the terminal two nucleotides of the 5′ or 3′ terminus. In one embodiment, the mesyl linkage is located at position −6. In one embodiment, the mesyl linkage is located at position−5. In one embodiment, the mesyl linkage is located at position +18. In one embodiment, the mesyl linkage is located at position +19. In one preferred embodiment, the mesyl linkage is located at position −2. In one preferred embodiment, the mesyl linkage is located at position −7. In one preferred embodiment, the mesyl linkage is located at position +4. In one preferred embodiment, the mesyl linkage is located at position +13. In one preferred embodiment, the mesyl linkage is located at position +21. In one preferred embodiment, the mesyl linkage is located at position +23.

Accordingly, in one embodiment, the oligonucleotide comprises mesyl linkages at positions +24, +23, +13, −2, −7 and 8. In one embodiment, the oligonucleotide comprises mesyl linkages at positions +24 +21, +13, +4, −7 and −8. In one embodiment the oligonucleotide comprises mesyl linkages at positions +24, +23, +21, +13, +4, −2, −7 and −8. In one embodiment, the oligonucleotide comprises mesyl linkages at positions +24, +23, −2, −7 and −8. In one embodiment, the oligonucleotide comprises mesyl linkages at positions +24, +23, −7 and −8. In one embodiment, the oligonucleotide comprises mesyl linkages at positions +24, −2, and −8. In one embodiment, the oligonucleotide comprises mesyl linkages at positions +24 and −8.

Alternatively, or additionally mesyl linkages may be located at the terminal nucleotides of the ASO of the invention, i.e., between the terminal and penultimate nucleotide of the 5′ and/or 3′ end of the ASO. In one embodiment, a mesyl linkage is located in the 5′ and/or 3′ flanking regions of the ASO. In one embodiment, a mesyl linkage is located at position +24 and/or at position −8.

The chemically modified oligonucleotides of the invention may be symmetrical, which means that the two nucleotide sequences adjacent to the CBT have the same length, or not symmetrical (asymmetrical or asymmetric design), which means that the two sequences flanking the CBT, i.e., the regions 5′ and 3′ to the CBT and/or position N₀, have different lengths. The asymmetric design enables a more flexible use of the sequence space around the target. Hence, in one embodiment, the oligonucleotide comprises an asymmetric design. In one embodiment, the oligonucleotide has: (i) a length of 20 to 29 nt located 5′ to N₀, and (ii) a length of 5 to 20 nt located 3′ to N₀.

The oligonucleotides of the invention may be of any length suitable to achieve an edit. The oligonucleotides of the invention are preferably at least 22, more preferably at least 26 nucleotides (nt) long, at least 27 nucleotides long, at least 30 nucleotides long, at least 35 nucleotides long. In some instances, the oligonucleotides may range from about 25-80 nt in length, e.g., about 25-39 nt, about 40-60 nt or about 61-80 nt in length. In one embodiment, the oligonucleotide has a length of 25-80 nt. In one embodiment, the oligonucleotide has a length of 25-50 nt. In one embodiment, the oligonucleotide has a length of 25, 26, 27, 28, 29, 30, 31, 32, 33, 34, 36, 36, 37, 38, 39, 40, 41, 42, 43, 44, 45, 46, 47, 48, 49, 50, 51, 52, 53, 54, 55, 66, 57, 58, 59, 60, 61, 62, 63, 64, 65, 66, 67, 68, 69, 70, 71, 72, 73, 74, 75, 76, 77, 78, 79, or 80 nt. In one embodiment, the oligonucleotide has a length of 27 nt. In one embodiment, the oligonucleotide has a length of 30 nt. In one embodiment, the oligonucleotide has a length of 33 nt. In one embodiment, the oligonucleotide has a length of 34 at In one embodiment, the oligonucleotide has a length of 38 nt. In one embodiment, the oligonucleotide has a length of 44 nt. In one embodiment, the oligonucleotide has a length of 45,nt. In certain embodiments, the oligonucleotide has a length of 27, 30, 32, 34, 38, or 38, nt. In one embodiment, the oligonucleotide has a length of 30-40 nt. In some embodiments, the oligonucleotide has a length of 30-38 nt. In some embodiments, the oligonucleotide has a length of 30-34 nt. In some embodiments, the oligonucleotide has a length of 34-38 nt or 38 nt. In one embodiment, the oligonucleotide has a length of no more than 30, 31, 32, 33, 34, 35, 36, 37, or 38 nt, in one embodiment, the oligonucleotide has a length of no more than 38, 39, 40, 41, 42, 43, 44, or 45nt. Ranges and lengths intermediate to the above recited ranges and lengths are also contemplated to be part of the invention.

Without being bound by any theory, inventors submit that the ideal asymmetry for each target might depend on the length and the specific underlying sequence of the particular oligonucleotide. The inventors previously showed that for asymmetric ASOs, a shorter overall oligoribonucleotide is sufficient for high editing efficacy compared to the symmetric design (WO 2022/253810). It is known that ADAR works as an asymmetric dimer with a footprint of up to 50 bp. While some substrates are more efficiently edited by the deaminase domain alone rather than by the full-length protein, the opposite holds true for other substrates. This suggests that depending on the size of the target/drug RNA helix, ADAR might bind in different ways. This leads to a situation, wherein, depending on the length of the ASO, specific (a) symmetries on the target adenosine and specific modifications patterns (e.g., sugar and internucleoside linkage modifications) are preferred. For an optimal binding of the deaminase, a short 3′ terminus seems to be sufficient (at least 5 nt beside the CBT). On the other hand, the 5′ terminus may provide binding space for the dsRBDs and thus typically requires more nucleotides (at least 19 nt beside the CBT).

Some well-working embodiments of asymmetries provided herein and identified by the inventors of the instant application are listed in Table A below As previously described, the oligonucleotides of the invention have the following structural scheme: (length of 5 terminus)-(1)-(length of 3 terminus), wherein 1 corresponds to the central nucleotide of the CBT opposite of the target A. For example, an ASO of the invention with a length of 38 nt and an asymmetry of “29-1-8”, has a 5′ terminus that is 29 nt long and a 3′ terminus that is ant long. In one embodiment, the oligonucleotide is asymmetric. In one embodiment, the oligonucleotide has any one of the asymmetries listed in Table A.

TABLE A
Asymmetries of exemplary ASO designs according to the invention

| Exemplary | Length | Structural scheme/Asymmetry | SEQ |
|---|---|---|---|
| Construct | (nt) | (5′ terminus−1−3′ terminus) | ID NO: |
| AI-0067 | 38 | 29−1−8 | 40 |
| AI-0083 | 36 | 23−1−12 | 36 |
| AI-0949 | 34 | 25−1−8 | 146 |
| AI-0099 | 32 | 25−1−6 | 14 |
| AI-0107 | 30 | 23−1−6 | 8 |
| AI-1685 | 27 | 20−1−6 | 204 |

According to the invention, the AGO may be asymmetric. Hence, in one embodiment, the chemically modified oligonucleotide of the invention comprises an asymmetric design, wherein there is a different number of nucleotides 5′ and 3′ of N₀. For instance, there may be 20-30 nt at the 5′ terminus (5′ to N₀) and 5-20 nt at the 3 terminus (3′ to N₀). Alternatively, in one embodiment, there are 26 nt 5′ to N₀, and 6 nt 3′ to N₀. In some embodiments, there are up to 29 nt 3′ of the CBT. In some embodiments, there are no more than 29 nt 3′ of the CBT. In some embodiments. the 3′ terminus is shortened to a length of 5 nt 3′ of the CBT. In some embodiments, the 3′ terminus is shortened to a length of 4 nt 3′ of the CBT. In one embodiment, the region 3′ to the CBT contains 4, 5, or 6 nt. In some embodiments, there are 4-30 nt 5′ of the CBT. In one embodiment, there are no more than 30 nt 5 of the CBT, In one embodiment, the 5′ terminus is shortened to a length of 28 nt 5′ of the CBT. In one embodiment, the region 5′ to the CBT contains 22, 23, 24, 25, or 26 nt.

The oligonucleotides of the invention may have specific asymmetries In some embodiments, the oligonucleotide has an asymmetry as listed in Table A. In a preferred embodiment, the oligonucleotide has an asymmetry of 25-1-8 in a 5′ to 3′ direction.

The inventors have further realised that the length of the oligonucleotide can be shortened without losing its editing efficacy provided the oligonucleotide comprises additional 2′-sugar and internucleoside linkage modifications. The oligonucleotide may have a length of 26 nt to 38 nt. In one embodiment, the oligonucleotide comprises a length of 27 nt to 35 nt. In a preferred embodiment, the oligonucleotide has a length of 27 nt, 30 nt, 33 nt, or 34 nt. In one embodiment, the oligonucleotide comprises an asymmetric design, wherein at least 20 nt are 5′ to N₀, and wherein at least 5 nt are 3′ to N₀. In one embodiment, the oligonucleotide comprises an asymmetry of: a) 25-1-8; b) 29-1-8; c) 27-1-6; 0) 26-1-6; e) 23-1-6; or f) 20-1.6 and wherein the oligonucleotide comprises at least four 2′-F modifications. In one embodiment, the oligonucleotide comprises an asymmetric design, wherein at least 20 nt are 5′ to N₀, and wherein at least 5 nt are 3′ to N₀. In one embodiment, the oligonucleotide has an asymmetry of 25-1-8 and comprises between 5 and 20 2′-F modifications.

In one preferred embodiment, the oligonucleotide has an asymmetry of 25-1-8 in a 5 to 3′ direction. In one preferred embodiment, the oligonucleotide has an asymmetry of 25-1-8 in a 5′ to 3′ direction, and wherein the mesyl linkage is located at positions +24,-2, and-8. In one preferred embodiment, a further mesyl linkage is located at position +4 and/or position +13. In one preferred embodiment, a further mesyl linkage is located at position +21, and optionally at position −7 and −23.

Furthermore, the ASO of the invention may comprise 2″-fluoro (2′-F) and/or 2′Ome modifications. In one embodiment, at least 20%, 30%, 40%, 50% or 60% nucleotides are fluoro (F)-modified at the 2′ position of the sugar residue. In one embodiment, the oligonucleotide comprises 5 to 20 2′-F modifications. In one embodiment, the oligonucleotide comprises 12 2′-F modifications. In one embodiment, a 2′F modification is located at one or more of the following positions selected from he group consisting of: +22, +21, +19, +17, +16, +15, +14, +13, +11, +9, +8, +7, +6, +5, +3, +2, +1, and −8. In one embodiment, the 2′-F modification is located at position +22, +21, +19, +16, +15, +13, +11, +9, +7, +5, +2, and −3.

In one embodiment, at least 20%, preferably 30-70%, more preferably 40-60% of the chemical modifications outside the CBT are 2′-O-methyl (2′-OMe) substituents.

In one embodiment, each RNA nucleoside is replaced by either a 2′-modified RNA or DNA. In addition to the at least one linkage that is a methanesulfonyl (mesyl) linkage the oligonucleotide may comprise a phosphodiester (PO) linkage and/or internucleoside linkage modifications such as phosphorothioate (PS) or phosphoryl guanidine (PN) linkages. In one embodiment, the oligonucleotide comprises one or more internucleoside linkages selected from the group consisting of PN, PO and PS. In one embodiment, the further Internucleoside linkage is a PS linkage. In one embodiment, at least 40% of linkages are PS linkages. In one embodiment, between 40% and 65% of linkages are PS linkages. In one embodiment, the internucleoside linkage modification is a 3-3′ or 5′-5′ phosphate ester bonds (3′-P-3′ and 5′P-5′). In one embodiment, the natural 3′-5′ phosphodiester linkage is replaced by modified internucleoside linkages. In some embodiments, the naturally occurring one or more PO linkages are replaced by modified internucleoside linkages to introduce one or more PS linkages of non-phosphorus derived internucleoside linkages. In one embodiment, an internucleoside linkage is a PS linkage. In one embodiment, an internucleoside linkage is a stereorandom PS linkage. In one embodiment, an internucleoside linkage is a chirally controlled PS linkage. In one embodiment, an internucleoside linkage is not a chirally controlled PS linkage.

In one embodiment, less than 60%, 50%, 45%, 40% of the internucleoside linkages are PO linkages. In one embodiment, less than 30% of the internucleoside linkages are PO linkages. In one embodiment, no more than 90%, 80%, 70%, 60%, 50%, 40%, 30%, or 20% of the internucleoside linkages are PS linkages. In one embodiment, (I) less than 60%, 50%, 45%, 40%, preferably less than 30% of the internucleoside linkages are PO linkages; and/or (ii) no more than 90%, 80%, 70%, 60%, 50%, 40%, 30%, or 20% of the internucleoside linkages are PS linkages. In one embodiment, the oligonucleotide comprises a PO linkage at position +22, +17, +16, +11, +8, +5, +3, +2 and/or −3. In one embodiment, there is a) no mesyl linkage at position −3; and/or b) no PN linkage at position +24, +21, +13, +4, −2, and/or −8.

In addition to the design features mentioned before, the chemically modified oligonucleotides may comprise at least one nucleotide of the CBT modified at the 2′-position of the sugar base or being deoxyribonucleosides, which permits added stabilization against nuclease digestion. Hence, in certain embodiments, the CBT is chemically modified. The CBT (5′ . . . -N₊₁-N₀-N₋₁- . . . 3′) may carry different modifications and permutations of the various modifications. That is, positions N₊₁, N₀ and/or N₋₁ may carry modifications at the 2′ position. In one embodiment, only one position within the CBT is chemically modified. In one embodiment, two positions within the CBT are chemically modified. In one embodiment, all positions within the CBT are chemically modified.

In one embodiment, at least one of the three oligonucleotides of the CBT is a deoxyribonucleotide. In one embodiment, at least one of the three nucleotides of the CBT is chemically modified at the 2′ position of the sugar residue. in one embodiment, at least one of the three oligonucleotides is 2′-FANA-modified. In one embodiment, at least one of the three oligonucleotides is-O-methyl-modified. In one embodiment, at least one of the three oligonucleotides is 2′-F-modified. In some embodiments, at least one of the three nucleotides of the CBT is chemically modified at the 2′-position of the sugar residue, a deoxyribonucleoside, or a combination thereof. In one embodiment, the chemical modification at the 2′ position is one or more of the following: (i) N₊₁ is 2′fluoro (2′-F), 2′ fluoroarabinoside (2′-FANA). deoxyribonucleic acid (DNA), 2′-O-Methoxyethyl (2′-MOE) or 2′-O-Methyl (2′-OMe): and/or (ii) N₀ is 2′-FANA or DNA; and/or (iii) N₋₁ is 2′-FANA, DNA or 2′-OMe. In one embodiment, N₋₁ is 2′-OMe. In one embodiment, (i) N₊₁ is 2′-O-Methoxyethyl (2′-MOE); (ii) N₀ is DNA; and (iii) N₋₁ is DNA.

In one embodiment, at least two of the three nucleotides of the CBT are chemically modified at the 2′ position of the sugar residue, a deoxyribonucleoside, or a combination thereof. In some embodiments, N₊₁ is 2′-F. 2′-FANA, DNA, or 2′-OMe; and/or N₀ is 2′-FANA or DNA; and/or N₋₁ is 2′-FANA, ONA, or 2′-O-methyl. In one embodiment, N₊₁ is DNA. In one embodiment, N₊₁ is 2′-F. In one embodiment, N₊₁ is 2𝔍-FANA. In one embodiment, N₀ is 2′-FANA. In one embodiment, N₀ is DNA. In one embodiment, N₋₁ is 2′-FANA. In one embodiment, N₋₁ is DNA.

According to one embodiment, each of the three nucleosides of the CBT is either singularly or a combination of: (a) a deoxyribonucleotide; and/or (b) 2′-fluoroarabinoside (2′-FANA) modification; and/or (c) 2′O-methyl (2′-OMe) modification; and/or (d) 2′-fluoro (2′-F) modification.

In one embodiment, the middle or centre nucleotide (Na) of the CBT does not comprise a 2′-sugar modification, although it may be a deoxyribonucleotide. In one embodiment, N₀ does not comprise a 2′-alkyl modification. In one embodiment, N₀ does not comprise a 2′-OMe modification.

In some embodiments, the CBT comprises no cytosine analogues. In one embodiment, the CBT does not comprise pseudoisocytidine (PIC) or 6-amino-5-nitro-2(1H)-pyridone. In one embodiment, the CBT does not comprise a Benner's base Z (dZ). In other embodiments, the CBT does not comprise a cytidine analogue such as, for example, 5-hydroxyC—H+, 5-aminoC—H+ and 8-oxoA (syn), Hence, in one embodiment, (i) N₀ comprises no 2′-sugar modification, preferably wherein N₀ comprises no 2′-alkyl modification (e.g., no 2′OMe modification), and/or (ii) the CBT comprises no cytosine analogues.

The oligonucleotides of the invention may also comprise modifications to the nucleotides positioned outside of the CBT. For example, the sugar or base of the one or more nucleotides may be modified. This is typically to provide greater resistance to nuclease attack in vivo. In one embodiment, the oligonucleotide incorporates modifications at one or more of the 2′-position of the nucleotides and these modifications are composed of different groups. In one embodiment, the oligonucleotide comprises a mixture of 2′-O-alkyl, 2′-F, 2′-MOE, 2′-FANA and/or LNA modifications. The oligonucleotides may comprise any permutation of these 2-sugar modifications.

In one embodiment, at least 50%, more preferably at least 80% of the nucleotides outside the CBT are modified independently from another at the 2′ position of the sugar residue. In one embodiment, the 2′-sugar modification is selected from 2′-F, 2′-FANA, 2′-O-alkyl, 2′-O-methoxyethyl (2′-MOE), and/or locked nucleic acid (LNA). In one embodiment, the 2′-O-alkyl modification is a 2′-OMe modification However, the oligonucleotides of the invention preferably do not contain blocks of more than 6 continuous nucleotides modified in the same way. In one embodiment, the oligonucleotides preferably do not contain blocks of more than 6 continuous 2′-OMe- or 2′-F-modified nucleotides. In one embodiment, the oligonucleotides preferably do not contain blocks of more than 5, 4, or 3 continuous 2′-OMe- or 2′-F-modified nucleotides. In one embodiment, the oligonucleotides preferably do not contain blocks of more than 4 continuous 2′-OMe- or 2′-F-modified nucleotides

In one embodiment, a 2′-sugar modification is a 2′-O-alkyl modification. In one embodiment, a 2′-O-alkyl modification is a 2′-OMe, 2′-O-ethyl, or 2′-O-propyl modification. In some embodiment, a 2-sugar modification is a 2′-MOE modification In one embodiment, a 2′-sugar modification is 2′-OMe. In some embodiments, a 2′-sugar modification is 2′-MOE. In some embodiments, a 2′-sugar modification is 2′-OR, wherein R is substituted C1-10 aliphatic. In some embodiments, a 2′-sugar modification is 2′-F. In some embodiments, a 2′-sugar modification is 2′-FANA.

In a preferred embodiment, a mixture of 2′-F- and 2′-O-alkyl-modifications is beneficial to editing and that a minimum of 10% of each is desirable. In some embodiments, the oligonucleotide comprises a mixture of 2′-F- and 2′-O-alkyl-modifications and a minimum of 15% of each 2′-F-and 2′-O-alkyl-modifications. In some embodiments, the oligonucleotide comprises a mixture of 2′-F- and 2′-O-alkyl-modifications and a minimum of 20% of each 2′-F-and 2′-O-alkyl-modifications In some embodiments, the oligonucleotide comprises a mixture of 2′-F- and 2′-O-alkyl-modifications and a combined minimum of 15%-20%, 20-30%, 30%-40%, 40-50% or 40-60% of 2′-F- and 2′-O-alkyl-modifications.

In some embodiments, the oligonucleotide comprises at least 10% of 2′-F, 2′-OMe, 2′-MOE and/or 2′-FANA modifications. In some embodiments, the oligonucleotide comprises at least 15%, 20%, 25%, 30%, 35%, 40% of 2 F, 2′-OMe, 2′-MOE or 2′-FANA modifications. In some embodiments, the oligonucleotide comprises at least 15%, 20%, 25%, 30%, 353%, 40% of 2′-F, 2′-OMe, 2′-MOE and 2′-FANA modifications.

The oligonucleotides of the invention may not carry a 2-sugar modification in some of the positions. In one embodiment, not all nucleotides comprise a 2′-alkyl modification. In some instances, the 2′-O-alkyl modification is not a 2′-MOE. In some instances, the 2′-modification is not a 2′-OMe, 2′-F or 2′-LNA modification. In some embodiments, not all 2′-sugar modifications are 2′-O-alkyl modifications. In some embodiments, not all 2′-sugar modifications are 2′-F modifications. In some embodiments, not all 2′-sugar modifications are 2′-MOE modifications.

The oligonucleotides of the invention may comprise RNA and/or DNA. Also, the oligonucleotides may comprise modifications at the 2′-position of the sugar residue. In one embodiment, 20-100%, 30-100%, 40-100%, 50-100%, 60-100%, 70-100%, 80-100%, or 90-100% of nucleotides are DNA or 2′-modified. In one embodiment, 20-100% of nucleotides are DNA or Z′-modified. In one embodiment, 50-100% of nucleotides are DNA or 2′-modified nucleotides. In one embodiment, 100% of nucleotides are DNA or 2′-modified nucleotides. In one embodiment, 30-95%, 40-95%, 40-90%, 60-95%, 50-90%, 60-95% or 60-90% of nucleotides are DNA or 2′-modified nucleotides. In some embodiments, the DNA content of the oligonucleotide is between 0-10%. In one embodiment, the DNA content is between 1-9%, preferably between 1-78%. In one embodiment, the DNA content is between 1-6%, Preferably between 1-5%. In one embodiment, the DNA content is between 1-4%, optionally between 1-3%. In one embodiment, the DNA content is less than 10%, 9%, 8%, 7%, 6%, 5%, 4%, or 3%.

In one embodiment, no more than 80%, 80%. 70%, 60%, 50%, 40%, 30%, 20% or 10% of nucleotides outside the CBT are deoxynucleotides. In some embodiment, no more 10%, optionally no more than 8%, optionally no more than 6% of nucleotides outside the CBT are deoxynucleotides. In one embodiment the above percentages are satisfied with only 2′-modified nucleotides and no ONA. In some embodiments, the oligonucleotide comprises no DNA. In one embodiment, only 1 nucleotide outside the CBT is deoxynucleotide. In one embodiment, no more than 2 nucleotides outside the CBT are deoxynucleotides. In one embodiment, no more than 4 nucleotides outside the CBT are deoxynucleotides. In one embodiment, no more than 3 nucleotides outside the CBT are deoxynucleotides. In one embodiment, no more than 5 nucleotides outside the CBT are deoxynucleotides. In one embodiment. no more than 6 nucleotides outside the CBT are deoxynucleotides. In some embodiment, no more than 7 nucleotides outside the CBT are deoxynucleotides.

The oligonucleotides of the invention may specifically comprise 2′-F and/or 2′-OMe modifications. In one embodiment, the oligonucleotide comprises one or more 2′-F modifications. In one embodiment, no more than 5%, 10%, 20%, 30%, 40%, 50%, 80%, or 70% of nucleotides are 2′-F-modified. In one embodiment, at least 5%, 10%, 20%, 30%, 40%, 50%, or 60% of nucleotides are 2′ F-modified. In one embodiment, no more than 35% of nucleotides are 2′-F modified. In one embodiment, 30-60% of nucleotides are 2′-F-modified. In one embodiment, 20-70%, preferably 30-45%, of nucleotides are 2′-F-modified. In one embodiment, 35-65% of nucleotides are 2′-F-modified.

Oligonucleotides may also comprise 2′-O-methyl (2′-OMe) modifications. In one embodiment, the oligonucleotide comprises one or more 2′-OMe modifications. In one embodiment, no more than 5%, 10%, 20%, 30%, 40%, 50%, 60%, or 70% of nucleotides are 2′-OMe-modified. In one embodiment, 20-60% of nucleotides are 2′-OMe-modified. In one embodiment, 5-55%, preferably 25-55% of nucleotides are 2′-OMe-modified. In one embodiment, at least 20%, preferably 30-70%, more preferably 40-60% of the chemical modifications outside the CBT are 2′-O-methyl substituents.

The chemically modified oligoribonucleotide according to the invention may comprise a general core sequence of formula I: 5′- . . . N₊₅^{a}N₊₄^{b}N₊₃^{c}N₊₂^{d}N₊₁^{e}N₀^{f}N₋₁^{g}N₋₂^{h}N₋₃ⁱN₋₄^{j}N₋₅ . . . -3′ (formula 1). In this formula i there is a Central Base Triplet (CBT) of three nucleotides, whereby the central nucleotide is designated by “0”. The nucleotide designated as “0” and the two nucleotides directly adjacent to nucleotide “0” having the number −1 and +1 are designated as a Central Base Triplet, whereby the central nucleotide designated as “0” is directly opposite to the target adenosine in the target RNA. The nucleotide of formula I is flanked 5′- and (adjacent to nucleotide +5) and at the 3′-end (adjacent to nucleotide-4) with further oligonucleotide sequences, which may have either the same length of different lengths.

In one embodiment, the oligonucleotide comprises the following core sequence: 5′- . . . N₊₅^{a}N₊₄^{b}N₊₃^{c}N₊₂^{d}NN₊₁^{e}N₀^{f}N₋₁^{g}N₋₂^{h}N₋₃ⁱN₋₄^{j}N₋₅ . . . -3′, wherein at least linkages d and e are modified, optionally wherein (i) d and e are phosphorothioate (PS) linkages and whereby at least 2 linkages are phosphate (PO) linkages; and/or (ii) linkage h is not a PS linkage; and/or (iii) f and j are a PS linkage, and/or (iv) b is a PO or a PS linkage. It is also preferred that linkage g is a mesyl linkage and/or that linkage a is a mesyl linkage.

The positioning of additional, chemically distinct internucleoside linkages within the oligonucleotide of the invention plays an important role when determining a balance between high editing yields, a long half-life and cytotoxicity. To obtain improved stabilization and editing, oligonucleotide linkages may be modified at particular positions within the oligonucleotide sequence (formula I). In one embodiment, linkage a is a mesyl or PS linkage. In one embodiment, d and e are PS linkage modifications, optionally wherein f is an internucleoside linkage modification. In one embodiment, d and e are PS linkage modifications. In one embodiment, f is a PS linkage.

The inventors have found that placement of PO linkages at specific positions within the oligonucleotide stabilises the oligonucleotide and contributes to enhanced target editing. Accordingly, in one embodiment, there is a PO linkage at position +3 and position −3 of the oligonucleotide, i.e., linkages b and h are PO linkages. In one embodiment, linkage h is not chemically modified, i.e., linkage h is a PO. In one embodiment, linkage h is a PO linkage. In one embodiment, linkage i is chemically modified. In some embodiments, linkage i is a PS linkage. In one preferred embodiment, h is a PO linkage and i is a PS linkage. In one embodiment, linkage i is a PS linkage. In one embodiment, up to three linkages selected from the group consisting of linkages b, c, f, g and) are also PS linkages. It is, however, preferable that not all linkages a to j are PS linkages. In especially preferred embodiments, the linkage f is a PS linkage. In especially preferred embodiments, linkages d and e are PS linkages whereas linkage h is a PO linkage. In one embodiment, b is a PO or a PS linkage. In one embodiment, b and h are PO linkages In a preferred embodiment, linkage a is a mesyl linkage.

In one embodiment, linkage d and e are PS linkages, linkage h is a PO linkage, linkage i is a PS linkage, and linkage g is a mesyl linkage.

Also, due to cytotoxicity and non-specific protein binding, it is desirable to reduce the overall PS content of the oligonucleotide. In one embodiment, at least linkages d and e are PS linkages and whereby at least 2 linkages are phosphate (PO) linkages. Also, PS linkages should be avoided at position h of the oligonucleotide sequence. PS linkages at such positions were found to impair editing strongly. In one embodiment, linkages h and i are not PS linkages, optionally wherein h and i are PO linkages. In another embodiment, linkage f, j, g and/or c are/is a PS linkage(s). In one embodiment, linkage g is a phosphate (PO) linkage. In one embodiment, linkage g is a 3′,5′-phosphodiester linkage. In one preferred embodiment, linkage g is a mesyl linkage.

Oligonucleotides of different lengths may require a different mixture of particular 2′-modifications and internucleoside linkage modifications in order to provide optimal RNA editing. The shorter the oligonucleotide, the better may be the endosomal escape. Moreover, cytotoxicity of the particular oligonucleotide may also depend on its length. Also, shorter oligonucleotides may experience higher specificity. On the other hand, while longer oligonucleotides may bind stronger or faster to their respective RNA target, editing-boosting bulges, mismatches and wobbles may also work better in long oligonucleotides. As a result, there is a benefit and/or trade-off for both long and short oligonucleotides of the invention.

The inventors found that in the oligonucleotides of the invention, a higher 2′-F content improved editing and could compensate for shortening of the overall length of the oligonucleotide. In some embodiments, at least 10%, 20%, 30%, 40%, 50% or 60% nucleotides are fluoro (F)-modified at the 2′ position of the sugar residue, optionally wherein the 2′-F modification is at one or more of the following positions: 29, 28, 25, 23, 21, 17, 15, 14, 13, 9, 7, 6, 5, 4, 3, 1, 3, −6, −7, −8, −10, −12, −13, −14, and −15. In one embodiment, the 2′-F modification is at one or more of the following positions: 29, 28, 25, 23, 21, 17, 15, 14, 13, 9, 7, 6, 4, 3, −8, −7, −8, −10, −12, −13, −14, and −16. In a preferred embodiment, the 2′-F modification is at one or more of the following positions: 29, 28, 23, 21, 15, 9, 7, 6, 5, 3, 1, −10, −13, −14, and −15. In one embodiment, the 2′-F modification is at one or more of the following positions: 28, 23, 21, 9, 1, −13 and −14. In some embodiments, about 10%-20%, 20%-30%, 30%-40%, or 50%-60% nucleotides are F-modified at the 2′ position of the sugar residue. In one embodiment, the oligonucleotide has a length of 30-40 nt and 4-20 2′-F modifications. In one embodiment, the oligonucleotide has a length of 30-38 nt and 2-19 2′-F modifications. In a preferred embodiment, the oligonucleotide has a length of 38 nt and 17 2′-F modifications. In a preferred embodiment, the oligonucleotide has a length of 34 nt and 19 2′-F modifications. In a preferred embodiment, the oligonucleotide has a length of 33 nt and 19 2′-F modifications.

In one embodiment, the oligonucleotide comprises an internucleoside linkage modification selected from the group consisting of PS, 3′-methylenephosphonate, 5′-methylenephosphonate, 3′-phosphoroamidate, 2′-5′-phosphodiester, and PN. In a preferred embodiment, the internucleoside linkage modification is a PS linkage. In one embodiment, the internucleoside linkage modification is a 3′-methylenephosphonate linkage. In one embodiment, the internucleoside linkage modification is a 5′-methylenephosphonate linkage. In one embodiment, the internucleoside linkage modification is a 3′-phosphoroamidate linkage. In one embodiment, the internucleoside linkage modification is a 2′5′-phosphodiester linkage. In one embodiment, the internucleoside linkage modification is a PN linkage. In one embodiment, the nucleic acid analogue is a PNA (peptide nucleic acid). In one embodiment, the nucleic acid analogue is PMO (phosphorodiamidate linked morpholino). In one embodiment, the at least one internucleoside linkage modification is PS. In one embodiment, the oligonucleotide contains a continuous stretch of PS linkages. In one embodiment, the continuous stretch of PS linkages is 10, 11, 12, 13, 14, 15, 16, 17, 18, 19, 20, 21, 22, 23, 24 or more linkages long.

2′-MOE residues are used for splice switching oligonucleotides and typically have very low cytotoxicity. However, due to their bulkiness they are not well accepted in larger quantities. The inventors of the invention have realized that 2′-MOE modifications could be placed 5′ and 3′ of the CBT and/or at the termini of the oligonucleotides without reducing the overall editing of the ASO. Specifically, the inventors realised that the amount of 2′-MOE modifications could be limited to about no more than about 6, 7, or 8 nucleotides to still obtain good RNA editing. Therefore. the oligonucleotide may comprise no more than 6, 7, or 8 2′-MOE modifications. In one embodiment, the oligonucleotide comprises 2′-MOE terminal blocks at the 5′ and 3′ termini, wherein at each terminus there are no more than 4 nucleotides with 2′-MOE, preferably no more than 3 nucleotides with 2′-MOE. In one embodiment, at each terminus there are no more than 4 nucleotides with 2′-MOE, preferably no more than 3 nucleotides with 2′-MOE. In one embodiment, the oligonucleotide comprises 2′-MOE terminal blocks at the 5′ and 3′ termini, wherein at each terminus there are no more than 4 nucleotides with 2′-MOE. In one embodiment, the oligonucleotide comprises 2′-MOE terminal blocks at the 5′ and 3′ termini, wherein at each terminus there are no more than 3 nucleotides with 2′-MOE. Notably, 2′-MOE modification may also be located at internal positions within the ASOs of the invention. In one embodiment, there is a 2′-MOE at position +17. In one embodiment, there is a 2″-MOE at position +14 in one embodiment, there is a 2′-MOE at position +12. In one embodiment, there is a 2′-MOE at position +8. In one embodiment, there is a 2′-MOE at position +6. In one embodiment, there is a 2′-MOE at position +3. In one embodiment, there is a 2′-MOE at position +1. In one embodiment, there is a 2′-MOE at position −4. In one embodiment, there is a 2′-MOE at positions +17, +14, +8, +6, +3, +1, and −4.

In one embodiment, the oligonucleotide does not comprise any 2′-O-methoxyethyl (2′-MOE) modifications at the outermost three nucleotides of the 3′ terminus and/or the 5′ terminus.

In one embodiment, the oligonucleotide comprises an iso-uridine (SbU) modification, optionally wherein the SbU modification is at position zero (0; N₀).

In one embodiment, the oligonucleotide does not comprise any PN modifications at the outermost three nucleotides of the 3′ terminus and/or the 5′ terminus.

In one embodiment, the oligonucleotide does not comprise any methylphosphonate (MP) linkage modifications.

Locked nucleic acid (LNA) is a structurally rigid modification that increases the binding affinity of a modified oligonucleotide. In one embodiment, the oligonucleotide comprises terminal LNAs, wherein the oligonucleotide comprises 2 to 5 LNAs at each terminus. In one embodiment, the oligonucleotide comprises 2 LNAs at each terminus. In one embodiment, the oligonucleotide comprises 2 LNAs at the 5′ terminus. In one embodiment, there is no 2′-MOE modification within the CBT. Oligonucleotides may have a general structure of (length of 5′ terminus)-(1)-(length of 3′ terminus), wherein 1 corresponds N₀ or to the central nucleotide of the CBT opposite of the target A. The region 3′ to N₀ is referred to as the “3′ flanking region” or “3′ terminus flanking region”. The region 5′ to N₀ is referred to as the “5′ flanking region” or “5′ terminus flanking region”. In one embodiment, the 3′ terminus flanking region comprises the terminal 6, 5, 4, 3, 2 or 1 nucleotide(s) the 3′ end of the oligonucleotide; and the 5′ terminus flanking region comprises the terminal 10, 9, 8, 7, 6, 5, 4, 3, 2 or 1 nucleotide(s) of the 5′ end of the oligonucleotide. In one embodiment, the 5′ terminal flanking region(s) is the outermost 1-5, 1-6, 1-7, 1-8, 1-9, 1-10, 1-11, or 1-12 nucleotides. In one embodiment, the 5′ terminal flanking region(s) is the outermost 1-4, 1-5, 1-6, 1-7, or 1-8 nucleotides.

Uniform blocks or stretches of large 2′-sugar modifications within the ASO tend to interfere with the binding of ADAR's dsRNA binding proteins (dsRBDs). Hence, the oligonucleotides of the invention may be modified in a way to avoid such interference. For example, the oligonucleotides are modified such that they do not comprise continuous stretches or uniform blocks of nucleotides carrying the same chemical modification (i.e., avoidance of a block-like modification structure). Avoiding uniform blocks of more than 6 nucleotides with the same 2′-modification prevented a strong loss of editing activity with natural ADARs. Hence, in one embodiment, the oligonucleotide is not uniformly modified. In one embodiment, the oligonucleotide contains no uniform blocks and/or no block-like modification structure. In one embodiment, the oligonucleotide does not comprise continuous stretches or uniform blocks of nucleotides carrying the same chemical modification at the 2′ position of the sugar moiety. A “block” or “stretch” may, e.g., not comprise more than 4, 5 or 6 nucleotides with the same 2′-sugar modification. In some instances, the block or stretch may be shorter or longer. In one embodiment, the oligonucleotide contains only 1 block of no more than 6, 5, 4, or 3 nucleotides with the same 2′-sugar modification. In one embodiment, the oligonucleotide contains 2 blocks, separated by one or more oligonucleotides having a different 2′-sugar modification. In order embodiments, the oligonucleotides comprise at least 1 block of nucleotides with the same 2′-sugar modification. In another embodiment, the oligonucleotide comprises 1, 2, 3, or more blocks of nucleotides with the same 2′-sugar modification.

Specifically, stretches of more than 6 nucleotides with the same 2′-modification should be avoided. Avoiding uniform blocks of more than 6 nucleotides with the same 2′-modification prevented a strong loss of editing activity with natural ADARs. Hence, the oligonucleotides of the invention may be modified to not include uniform blocks or a continuous stretch of the same 2′ sugar modification. In one embodiment, the oligonucleotide comprises one or more 2′-sugar modifications. optionally wherein no more than 6 consecutive nucleotides have the same 2′-modification. In one embodiment, no more than 6 consecutive nucleotides have the same modification. In one embodiment, no more than 4 consecutive nucleotides have the same modification. In one embodiment, no more than 3 consecutive nucleotides have the same modification. In one embodiment, no more than 2 consecutive nucleotides have the same modification. In one embodiment, less than 6, 5, 4, or 3 consecutive nucleotides have the same 2′-modification. Hence, in one embodiment, the 2′-sugar modification is 2′-deoxyribose (DNA). In one embodiment, no more than 6 consecutive nucleotides are 2′-H (DNA) modified. In one embodiment, no more than 5 consecutive nucleotides are 2′-H-modified. In one embodiment, no more than 4 consecutive nucleotides are 2′-H-modified. The 2′-sugar modification may be 2′-ribose. In one embodiment, no more than 6 consecutive nucleotides are 2′-H (DNA) modified. In one embodiment, no more than 5 consecutive nucleotides are 2′-H-modified. In one embodiment, no more than 4 consecutive nucleotides are 2′-H-modified. In one embodiment, no more than 6 consecutive nucleotides are 2′-F-modified. In one embodiment, no more than 5 consecutive nucleotides are 2′-F-modified. In one embodiment, no more than 4 consecutive nucleotides are 2′-F-modified. In one embodiment, no more than 6 consecutive nucleotides are 2′-O-alkyl-modified. In one embodiment, no more than 5 consecutive nucleotides are 2′-O-alkyl-modified. In one embodiment, no more than 4 consecutive nucleotides are 2′-O-alkyl-modified, optionally wherein no more than 4 consecutive nucleotides are 2′-OMe-modified. In one embodiment, the oligonucleotide comprises 2, 3, 4, 5, or 6 consecutive nucleotides with the same 2′-modification, e.g., 5 consecutive nucleotides are 2′-F-modified.

The oligonucleotide of the invention may contain some “continuous stretch(es)” or “uniform block(s)” of a certain length. In one embodiment, the size or length of the “continuous stretch(es)” or “uniform block(s)” is 2, 3, 4, 5, or 6 nucleotides long. In one embodiment, the size or length of the “continuous stretch(es)” or “uniform block(s)” is no more than 2, 3, 4, 5, or 6 nucleotides long. In one embodiment, the oligonucleotide comprises no more than 2, 3, 4, 5, or 6 consecutive nucleotides comprising a 2′-F modification. In one embodiment, the oligonucleotide comprises no more than 2, 3, 4, 5, or 6 consecutive nucleotides comprising a 2′-OMe modification. In one embodiment, one or more uniform blocks are interrupted. Interruption can take place by any other chemical modification (e.g., DNA, RNA, 2′-F, 2′-OMe, 2′-MOE, (LNA, etc.). In one embodiment, one or more uniform blocks of 2′-F-modified nucleotides are interrupted, preferably by 2′-OMe-modified nucleotides. In one embodiment, one or more uniform blocks of 2′-OMe-modified nucleotides are interrupted, preferably by 2′-F-modified nucleotides. In some embodiments the blocks are disrupted by DNA.

DNA oligonucleotides are relatively stable molecules, while RNA oligonucleotides are much more unstable due to their chemical structure. It is commonly known that RNA is subject to autocatalysis and degradation by RNases. To achieve the necessary stability of an oligonucleotide, the final oligonucleotide ideally should not contain any unmodified RNA nucleobases. In one embodiment, the oligonucleotide contains no unmodified RNA nucleobases. In one embodiment, the oligonucleotide contains more than 10%, 20%, 30%, 40%, 50%, 60%, 70%, 80%, or more than 90% modified RNA nucleobases. In one embodiment, the oligonucleotide comprises more than 90% modified RNA nucleobases. In one embodiment, the oligonucleotide contains less than 90%, 80%, 70%, 60%, 50%, 40%, 30%, 20%, or less than 10% unmodified RNA nucleobases.

The inventors surprisingly found that depending on the length and symmetry of the ASO, there are preferred combinations of 2′-sugar and internucleoside linkage modifications that improve editing. In particular, the inventors have found that combining 2′-F and/or 2′OMe modifications and mesyl linkage modifications in the 5′ and 3 flanking regions improves overall editing when compared to control and ASOs of the prior art. Accordingly, in one embodiment, the oligonucleotide comprises a mixture of 2′-F, 2′OMe and mesyl modifications. In one embodiment, the oligonucleotide comprises 2′F and/or 2′OMe modifications and mesyl linkages at the outermost 5′ and 3′ terminal ends. In one embodiment, the oligonucleotide comprises: (i) a 23-1-6 asymmetry and mesyl linkages at +22 and −6; (ii) a 25-1-8 asymmetry and mesyl linkages at +24 and −8; (iii) a 27-1-6 asymmetry and mesyl linkages at +26 and −6; (iv) a 23-1-12 asymmetry and mesyl linkages at +22 and −12; or (v) a 29-1-8 asymmetry and mesyl linkages at +28 and−8 Accordingly, respective to symmetry (or asymmetry), the mesyl linkages are positioned at the outermost nucleotide position.

In certain cases, the ASO targeting domain, or nucleobase opposite to the target nucleobase that is to be edited, comprises, one or more wobble bases to compensate for the variability in the target sequence. That is, the less stringent base-pairing requirement of the wobble base (e.g., G-U, I-A, G-A, I-U, I-C, etc.) allows the ASO to pair with more than just one target nucleic acid. Accordingly, in some embodiments, mismatches and/or wobbles enable targeting of different target nucleic acids. In one embodiment, the oligonucleotide comprises one or more additional mismatches, wobble base and/or bulges. In some embodiments, the oligonucleotides of the invention may contain bulges of 1, 2, 3 or more nucleotides. In one embodiment, the oligonucleotide comprises one or more mismatches, wobble base, and/or bulges with respect to its target, and/or a mismatch at N₀. In one embodiment, the oligonucleotide comprises one or more mismatches, wobble base, and/or bulges with respect to its target. In one embodiment, the oligonucleotide comprises a mismatch at N₀.

The targeting sequence of the artificial nucleic acid typically comprises a nucleic acid sequence complementary or at least partially complementary to a nucleic acid sequence in the target RNA. In some embodiments, the targeting sequence comprises a nucleic acid sequence complementary or at least 60%, 70%; 80%, 90%, 95% or 99% of a nucleic acid sequence in the target RNA.

While the oligonucleotides may comprise DNA and/or RNA, they may also comprise additional modifications. LNAs (or “ln”) improve the binding power of ASOs by preserving the nucleoside in a preferred sugar confirmation. However, this preorganisation of the sugar by the additional bridge also reduces flexibility. Double-stranded RNA (dsRNA) structures are strongly perturbed in the active site of ADAR (flip-out mechanism). LNA may interfere with this process and thus it is desirable to place any LNAs in positions that are not inside or too close to the CBT. In one embodiment, the oligonucleotide comprises one or more LNA(s). In one embodiment, the oligonucleotide does not comprise an LNA modification at the outermost position of the 5′ and/or 3′ terminal ends.

The artificial and chemically modified oligonucleotides of the invention are suitable for editing a wide variety of endogenous RNA transcripts, e.g., endogenous mRNAs of housekeeping genes as well as endogenous transcripts of disease-related genes such as, e.g., SERPINA1, ACTB, STAT1, LRRK2, CRB1, NLRP3, CTNNB1, PEX1, and PDE6A. For example, the SERPINA1 gene encodes serine protease inhibitor alpha-I antitrypsin (A1AT), which protects tissues from certain inflammatory enzymes, including neutrophil stastase. A deficiency in A1AT (alpha 1 antitrypsin deficiency, A1AD) can lead to excessive break down of elastin in the lungs by neutrophil elastase. This may lead to reduced elasticity in the lungs and subsequent respiratory complications, including emphysema and chronic obstructive lung disease (COPD). Mutant A1AT can also build up in the liver, resulting in cirrhosis and liver failure. Hence, in one embodiment, the target is SERPINA1. In one embodiment, the target is ACTB.

Oligonucleotides targeting SERPINA1 may have different length, asymmetries and modification pattern. In one embodiment, the oligonucleotide is selected from the group consisting of the sequences listed in Tables 1-14. In one embodiment, the ASO is selected from the group consisting of: a) AI-0731 (SEQ ID NO: 151), AI-0860 (SEQ ID NO: 107), AI-0943 (SEQ ID NO: 150), AI-0945 (SEQ ID NO: 147), AI-0947 (SEQ ID NO: 144), AI-0948 (SEQ ID NO: 148), AI-0949 (SEQ ID NO: 146), AI-0950 (SEQ ID NO: 149), AI-0991 (SEQ ID NO: 145); b) AI-1068 (SEQ ID NO: 192), AI-1071 (SEQ ID NO: 200), AI-1207 (SEQ ID NO: 197), AI-1208 (SEQ ID NO: 198), AI-1209 (SEQ ID NO: 199); c) AI-1442 (SEQ ID NO: 191), AI-1443 (SEQ ID NO: 193), AI-1444 (SEQ ID NO: 194), AI-1445 (SEQ ID NO: 195), AI-1446 (SEQ ID NO: 196); d) AI-1885 (SEQ ID NO: 204), AI-1686 (SEQ ID NO: 212), AI-1687 (SEQ ID NO: 205), AI-1902 (SEQ ID NO: 209), AI-1699 (SEQ ID NO: 201), AI-1700 (SEO) ID NO: 202); e) AI-1849 (SEQ ID NO: 210), AI-1850 (SEQ ID NO: 211), AI-1952 (SEQ ID NO: 214), AI-1953 (SEQ ID NO: 215), AI-1954 (SEQ ID NO: 216), AI 1701 (SEQ ID NO: 217); f) AI-1684 (SEQ ID NO: 203), AI-1689 (SEQ ID NO: 206), A-1690 (SEQ ID NO: 207), AI-1691 (SEQ ID NO: 208), AI-1901 (SEQ ID NO: 213), AI-3163 (SEQ ID NO: 218), AI-3165 (SEQ ID NO: 219); g) AI-2940 (SEO ID NO: 190), AI-2938 (SEQ ID NO: 188), AI-2934 (SEQ ID NO: 184), AI-2936 (SEQ ID NO: 186); and h) AI-2691 (SEQ ID NO: 220), AI-2692 (SEQ ID NO: 221), AI-2693 (SEQ ID NO: 222), AI-2694 (SEQ ID NO: 223), AI-2695 (SEQ ID NO: 224), AI-2696 (SEQ ID NO: 225), AI-2584 (SEQ ID NO: 226), AI-2698 (SEQ ID NO: 227), AI-2699 (SEQ ID NO: 228), AI-2703 (SEQ ID NO: 229), AI-2704 (SEQ ID NO: 230), AI-2705 (SEQ ID NO: 231), AI-2706 (SEQ ID NO: 232), AI-3008 (SEQ ID NO: 233), and AI-3059 (SEQ ID NO: 234).

The inventors have found that some of the modified oligonucleotides show better stability and editing efficacy. Hence, in one preferred embodiment, the oligonucleotide is AI-0849. In one embodiment, the oligonucleotide comprises the following sequence from 5 to 3′-(GalNAc)mG&mC*mCfC&fC*mA*fG*mCmoeAfG*fC*moeT&fU*mCfA*mG*fUmoe(MeC)*C*moe(MeC)fU&mUmoeTfC*moeT*C*dI&mUfC*moeG*mA*mU*mG&mG (SEQ ID NO: 146), wherein m=2′-OMe, &=mesyl linkage, *=phosphorothioate, moe=2′-MOE, f=2′=fluoro, I=inosine, d=2′-H (deoxyribose; DNA). MeC=5′-methylcytidine, and A, C, G, T, U=nucleobase.

In one preferred embodiment, the oligonucleotide is AI-0991. In one embodiment, the oligonucleotide comprises the following sequence from 5′ to 3′: mG&mC*mCfC&fC*mA*fG*mCmoeAfG*fC*moeT&fU*mCfA*mG*fUmoe(MeC)*fC*moe(MeC)fU&mUmoeTfC*moeT*dC*dI&mUfC*moeG*mA*mU*mG&mG (SEQ ID NO: 145), wherein m=2′-OMe, &=mesyl linkage, *=phosphorothioate, moe=2′-MOE, f=2′fluoro, MeC=5′-methylcytidine, I=inosine, d=2-H (deoxyribose: DNA), and A, C, G, T, U=nucleobase.

In one preferred embodiment, the oligonucleotide is AI-1068. in one embodiment, the oligonucleotide comprises the following sequence from 5′ to 3′: mG&mC*mCfC&fC*mA*fG*mCmoeAfG*fC*moeT&fU*mCfA*mG*FUmoe(MeC)*fC*moe(MeC)fU&mUmoeTfC*moeT*dC*dI&mUfC*moeG*mA*mU*mG&mG(GalNac) (SEQ ID NO: 192), wherein m=2′-OMe, &=mesyl linkage, *=phosphorothioate, moe=2′-MOE, f=2′-fluoro, MeC=5′-methylcytidine, I=inosine, d=2′-H (deoxyribose; DNA), and A, C, G, T, U=nucleobase; GalNac=N-acetylgalactosamine.

In one preferred embodiment, the oligonucleotide is AI-1208. In one embodiment, the oligonucleotide comprises the following sequence from 5′ to 3′: mG&mC*mCfC*fC*mA*G*mCmoeAfG*fC*moeT*fU*mCfA*mG*fUmoe(MeC)*fC*mo e(MeC)fU*mUmoeTfC*moeT*dC*dI&mUfC*moeG*mA*mU*mG&mG(GalNac) (SEQ ID NO: 198), wherein m=2′-OMe, &=mesyl linkage, *=phosphorothioate, moe=2′-MOE, f=2′-fluoro, MeC=5′-methylcytidine, i=inosine, d=2′-H (deoxyribose; DNA), and A, C, G, T, U=nucleobase; GalNac=N-acetylgalactosamine.

In one preferred embodiment, the oligonucleotide is AI-1445. In one embodiment, the oligonucleotide comprises the following sequence from 5′ to 3′: mG&mC*mCfC&fC*mA*fG*mCmoeAfG*fC*moeT&fUmCfAmG*fUmoe(MeC)*fCmoe(MeC)*fU&mUmoeTfC*moeT*C*dI&mUIfC*moeG*mA*mU*mG&mG(GalNac) (SEQ ID NO: 195), wherein m=2′-OMe, &=mesyl linkage. *=phosphorothioate, moe=2′-MOE, f=2′-fluoro, MeC=5′-methylcytidine, I=inosine, d=2″H (deoxyribose; DNA), and A, C, G, T, U=nucleobase; GalNac=N-acetylgalactosamine.

In one preferred embodiment, the oligonucleotide is AI-1446. In one embodiment, the oligonucleotide comprises the following sequence from 5′ to 3′: mG&mCmCfC&fC*mA*fG*mCmoeAfG*fC*moeT&fUmCfAmG*fUmoe(MeC)*fCmoe(MeC)fU&mUmoeTfC*moeT*dC*dI&mUfC*moeG*mA*mU*mG&mG(GalNac) (SEQ ID NO: 196), wherein m=2′-OMe, &=mesyl linkage, *=phosphorothioate, moe=2′-MOE, f=2′-fluoro, MeC=5′-methylcytidine, I=inosine, d=2′-H (deoxyribose; DNA), and A, C, G, T, U=nucleobase; GalNac=N-acetylgalactosamine.

In one preferred embodiment, the oligonucleotide is AI-1687. In one embodiment, the oligonucleotide comprises the following sequence from 5 to 3′: mG&mC*mCfC*fC*mA*FC*mCmoeAfG*fC*moeT*fUmCfAmG*Umoe(MeC)*fCmoe(MeC)fU*mUmoeTfC*moeT*dC*dI&mUfC*moeG*mA*mU*mG&mG(GalNac) (SEQ ID NO: 205), wherein m=2′-OMe, &=mesyl linkage, *=phosphorothioate, moe=2′-MOE, f=2′-fluoro, MeC=5′-methylcytidine, I=inosine, d=2′-H (deoxyribose; DNA), and A, C, G, T, U=nucleobase; GalNac=N-acetylgalactosamine.

In one preferred embodiment, the oligonucleotide is AI-1691. In one embodiment, the oligonucleotide comprises the following sequence from &' to 3′: mG&mC&mCfC&fC*mA*fG*mCmoeAfG*fC*moeT&fU*mCfA*mG*fUmoe(MeC)*fC*moe(MeC)fU&mUmoeTfC*moeT*dC*dI&mUfC*moeG*mA*mU&mG&mG(GalNac) (SEQ ID NO: 208), wherein m=2′-OMe, &=mesyl linkage, *=phosphorothioate, moe=2′-MOE, f=2′fluoro, MeC=5′-methylcytidine, I=inosine, d=2′-H (deoxyribose; DNA), and A, C, G, T, U=nucleobase; GalNac=N-acetylgalactosamine.

In one preferred embodiment, the oligonucleotide is AI-1901. In one embodiment, the oligonucleotide comprises the following sequence from 5′ to 3′: mG&mC*mCfC*fC*mA*fG*mCmoeAfG*fC*moeT&fUmCfAmG*fUmoe(MeC)*fCmoe(MeC)fU*mUmoeTfC*moeT*dC*dI&mUfC*moeG*mA*mU*mG&mG(GalNac) (SEQ ID NO: 213), wherein m=2′-OMe, &=mesyl linkage, *=phosphorothioate, moe=2′-MOE, f=2′-fluoro, MeC=5′-methylcytidine, I=inosine, d=2′-H (deoxyribose; DNA), and A, C, G, T, U=nucleobase; GalNac=N-acetylgalactosamine.

In one preferred embodiment, the oligonucleotide is AI-1954. In one embodiment, the oligonucleotide comprises the following sequence from 5′ to 3′: mG&mCmCfC*fC*mA*fG*mCmoeAfG*fC*moeT*fUmCfAmoeG*fUmoe(MeC)*fCmoe (MeC)fU*mUmoeTfC*moeT*dC*dI&mUfC*moeG*mAmU*mG&mG(GalNac) (SEQ ID NO: 216), wherein m=2′-OMe, &=mesyl linkage, *=phosphorothioate, moe=2′-MOE, f=2′-fluoro, MeC=5′-methylcytidine, i=inosine, d=2-H (deoxyribose; DNA). and A, C, G, T, U=nucleobase; GalNac=N-acetylgalactosamine.

Addition of mesyl linkages to the nucleotide backbone has an overall positive effect on the editing efficacy when compared to methylphosphonate (MP) internucleoside linkages. Accordingly, in one preferred embodiment, the oligonucleotide is AI-2840 (SEQ ID NO: 190). In one preferred embodiment, the oligonucleotide is AI-2938 (SEQ ID NO: 188). In one preferred embodiment, the oligonucleotide is AI-2934 (SEQ ID NO: 184). In one preferred embodiment, the oligonucleotide is AI-2936 (SEQ ID NO: 186).

In one preferred embodiment, the oligonucleotide is AI-3059 (SEQ ID NO: 234). In one preferred embodiment, the oligonucleotide is AI-3008 (SEQ ID NO 233). In one preferred embodiment, the oligonucleotide is AI-2705 (SEQ ID NO: 231),

In one preferred embodiment, the oligonucleotide is AI-3163 (SEQ ID NO: 218). In one preferred embodiment, the oligonucleotide is AI-3166 (SEQ ID NO: 219).

The oligonucleotides of the invention may be modified at their 5′ and/or 3′ termini. For instance, targeted delivery of oligonucleotides to liver hepatocytes using N-acetylgalactosamine (GalNAc) conjugates has previously described for, e.g., treating liver diseases, including Hepatitis B virus (HBV), non-alcoholic Fatty Liver Disease and genetic diseases (Debacker et al., 2020).

Hence, oligonucleotides of the invention may comprise a moiety, which enhances cellular uptake of the oligonucleotide, e.g. N-acetylgalactosamine (GalNAc). Hence, in some embodiments, the chemically modified oligonucleotide comprises a moiety or is conjugated to a moiety that enhances cellular uptake of the oligonucleotide. In one embodiment, the moiety enhancing cellular uptake is a N-acetyl galactosamine (GalNAc). In one embodiment, GalNAc is conjugated to the 3′ terminus of the oligonucleotide. In one embodiment, GalNAc is conjugated to the 5′ terminus of the oligonucleotide.

The chemically modified oligonucleotides according to the present invention show increased hydrophobicity, stability against degradation and an optimal chemical modification pattern to bind ADARs. The oligonucleotides according to the invention differ from the nucleic acid oligonucleotides disclosed in the prior art insofar that they do not require a loop-hairpin structured recruiting moiety specifically for recruiting a deaminase. The oligonucleotides of the present invention may or may not comprise a loop-hairpin structure. In one embodiment, the chemically modified oligonucleotide does not comprise a loop-hairpin structured recruiting moiety,

In some embodiments, the present disclosure provides oligonucleotides (and compositions thereof), that do not include chirally controlled oligonucleotides or compositions thereof. In one embodiment, an internucleoside linkage is not chirally controlled. In one embodiment, an internucleoside linkage is not a chirally controlled PS linkage. In one embodiment, the oligonucleotide does not comprise independently controlled chiral phosphates. In some embodiments, one of more internucleoside linkage is not independently chirally controlled. In some embodiments, at least 60%, 70%, 75%, 80%, 85%, 90%, 85%, 99% or all internucleoside linkages are not chiral internucleoside linkages.

### Compositions

The chemically modified oligonucleotides of the invention may be incorporated into compositions. Accordingly, provided herein is a composition containing the oligonucleotide(s) of the invention. In some embodiments, the compositions are pharmaceutical compositions. In the context of the invention, the term composition and pharmaceutic compositions are used interchangeably. Hence. in some embodiments, the present disclosure provides oligonucleotide compositions of oligonucleotides described herein, in one embodiment, the composition contains one or more oligonucleotides of the invention. In some embodiments, the present disclosure provides a composition comprising a plurality of oligonucleotides. As used herein, pharmaceutical composition means a substance or a mixture of substances suitable for administering to an individual. For example, a pharmaceutical composition may comprise one or more active pharmaceutical agents (such as an oligonucleotide) and a sterile aqueous solution. The compositions provided herein can be in any form that allows for the composition to be administered to a subject. The compositions may be used in methods of treating and/or preventing a genetic disorder, condition, or disease.

In one embodiment, the composition comprises an oligonucleotide of the invention or a pharmaceutically acceptable salt thereof. In one embodiment, a composition comprises an oligonucleotide of the invention in an admixture with a pharmaceutically acceptable carrier. In some embodiments, the pharmaceutically acceptable carrier can simply be a saline solution. This can be isotonic or hypotonic.

In one embodiment, the composition is for veterinary and/or human administration. In some embodiments, a pharmaceutical composition comprises one or more other therapies in addition to an oligonucleotide of the invention.

The amount of an oligonucleotide or composition which will be effective in the treatment and/or prevention of a disease or disorder will depend on the nature of the disease and can be determined by standard clinical techniques. Exemplary doses for oligonucleotides range from about 10 ng to 1 g, 100 ng to 100 mg, 1 μg to 10 mg, or 30-300 μg oligonucleotide, e.g., RNA, per patient. In one embodiment, the oligonucleotide is present at a concentration of 4 nM to 100 nM, optionally at 20 nM or 25 nM. In one embodiment, the oligonucleotide is present at a concentration of 0.8 nM. In one embodiment, the oligonucleotide is present at a concentration of 4 nM. In one embodiment, the oligonucleotide is present at a concentration of 20 nM. In one embodiment, the oligonucleotide is present at a concentration of 25 nM.

In certain embodiments, the compositions of the invention include diluents of various buffer content (e.g., Tris-HCl, acetate, phosphate), pH, and ionic strength, and additives such as detergents and solubilizing agents (e.g., Tween 80, Polysorbate 80), anti-oxidants (e.g., ascorbic acid, sodium metabisulfite), preservatives (e.g., Thimersol, benzyl alcohol), and bulking substances (e.g., lactose, mannitol). In some embodiments, the material is incorporated into particulate preparations of polymeric compounds such as polylactic acid, polyglycolic acid, etc. or into liposomes. In some embodiments, hyaluronic acid is used. Such compositions may influence the physical state, stability, rate of in vivo release, and/or rate of in vivo clearance of the oligonucleotides and/or derivatives and/or pharmaceutically acceptable salt thereof. In some embodiments, the compositions are in liquid form or in dried powder, such as lyophilized form.

In certain embodiments, the compositions additionally compose one or more salts, e.g., sodium chloride, calcium chloride, sodium phosphate, monosodium glutamate, and aluminium salts (e.g., aluminium hydroxide, aluminium phosphate, alum (potassium aluminium sulfate), or a mixture of such aluminium salts). In other embodiments, the compositions described herein do not comprise salts.

The pharmaceutical compositions can be included in a container, pack, or dispenser together with instructions for administration.

The oligonucleotides or compositions thereof can be tested for in vivo toxicity in animal models. For example, animal models, described herein and/or others known in the art, used to test the activities of active compounds can also be used to determine the in vivo toxicity of these compounds. For example, animals are administered a range of concentrations of active compounds. Subsequently, the animals are monitored over time for lethality, weight loss or failure to gain weight, and/or levels of serum markers that may be indicative of tissue damage. These in vivo assays may also be adapted to test the toxicity of various administration mode and/or regimen in addition to dosages.

### Prophylactic and Therapeutic Uses

The invention describes the use of chemically modified oligonucleotides and compositions comprising the same in the medical setting. specifically, for site-directed editing of a target RNA (e.g., binding to the target RNA via the targeting sequence and by recruiting to the target site a deaminase). The invention describes chemically modified oligonucleotides and compositions comprising said oligonucleotides for use in the treatment or prevention of a genetic disorder, condition, or disease as well as methods for treating or preventing a genetic disorder, condition, or disease. Site-directed editing may take place in vitro, in vivo or ex vivo.

A chemically modified oligonucleotide or composition comprising the same may be used in the treatment and/or prevention of a medical condition in one aspect provided herein is a chemically modified oligonucleotide of the invention or a composition of the invention for use in therapy. In another aspect provided herein is an oligonucleotide of the invention or a composition comprising the same for use in the treatment or prevention of a genetic disorder, condition, or disease. In some embodiments, the disease or disorder is selected form the group consisting of liver or metabolic diseases and/or cardiac or cardiovascular diseases associated with a gain-of-function (GOF) or loss-of-function (LOF) mutation. In one embodiment, the genetic disorder, condition, or disease is associated with a point mutation. For example, the SERPINA1 gens encodes serine protease inhibitor alpha-I antitrypsin (A1AT). A1AT protects tissues from certain inflammatory enzymes, including neutrophil elastase A deficiency in A1AT (alpha 1 antitrypsin deficiency, A1AD) can lead to excessive break down of elastin in the lungs by neutrophil elastase. This may lead to reduced elasticity in the lungs and subsequent respiratory complications, including emphysema and chronic obstructive lung disease (COPD). Mutant A1AT can also build up in the liver, resulting in cirrhosis and liver failure. Accordingly, in one embodiment, the genetic disorder, condition or disease is associated with a G-to-A mutation in the SERPINA1 gene. In one embodiment, the mutation is selected from SERPINA1 E342K. In one embodiment, the disease or disorder comprises the SERPINA 1 gene or an alpha-1-antitrypsin deficiency (A 1AD or AATD), optionally wherein the target protein is alpha 1 antitrypsin. In one embodiment, the mutation is the PIZ mutation (a1-antitrypsin deficiency).

The chemically modified oligonucleotide of the invention or the (pharmaceutical) composition may be administered, for example, orally in any orally acceptable dosage form including, but not limited to, capsules, tablets, aqueous suspensions, or solutions, or parenterally, e.g., by parenteral injection. In some embodiments, formulations suitable for parenteral administration comprise sterile aqueous preparations of at least one embodiment of the present disclosure, which are approximately isotonic with the blood of the intended recipient. The amount of oligonucleotide or composition to be administered, the dosage and the dosing regimen can vary from cell type to cell type, the disease to be treated, the target population, the mode of administration (e.g., systemic versus local), the severity of disease and the acceptable level of side activity. In some embodiments, the amount of oligonucleotides administered in a pharmaceutical composition is dependent on the subject being treated, the subject's weight, the manner of administration.

Various delivery systems can be used to deliver the oligonucleotides of the invention. An oligonucleotide according to the invention can be delivered as is, i.e., naked and/or in isolated form to an individual, through an organ, e.g., mucosa of the eye, or directly to a cell. Hence, in a preferred embodiment, the oligonucleotide of the invention is administered and delivered ‘as is’, also referred to as ‘naked’ When administering an oligonucleotide of the invention, it is preferred that the oligonucleotide is dissolved in a solution that is compatible with the delivery method. Such delivery may be in vivo, in vitro or ex vivo. Hence, depending on the disease, disorder or infection that needs to be treated, or on the cell, tissue or part of the body that needs to be reached by the oligonucleotides (e.g., in case of beneficial editing), a different administration route or delivery method may be selected. Examples for delivery when an oligonucleotide is not delivered naked, are delivery agents or vehicles such as nanoparticles, like polymeric nanoparticles, microparticles, micelles, liposomes, antibody-conjugated liposomes, cationic lipids, polymers, or call-penetrating peptides.

Use of an excipient or transfection reagents may be used in the delivery of each of the oligonucleotides or compositions to a cell and/or into a cell (preferably a cell affected by a G-to-A mutation or that wherein “beneficial editing” is to be achieved as outlined herein). Preferred are excipients or transfection reagents capable of forming complexes, nanoparticles, micelles, vesicles and/or liposomes that deliver each oligonucleotide or composition as defined herein, complexed or trapped in a vesicle or liposome through a cell membrane. Many of these excipients are known in the art. Suitable excipients or transfection reagents comprise polyethylenimine (PEI; ExGen500 (MBI Fermentas)), LipofectAMINE™ 2000 (Invitrogen), lipofectin™, or derivatives thereof, and/or viral capsid proteins that are capable of self-assembly into particles that can deliver each constituent as defined herein to a target cell.

Oligonucleotides of the invention may be linked to a moiety that enhances uptake of the ASO in cells. Examples of such moieties are cholesterols, carbohydrates, vitamins, biotin, lipids, phospholipids, call-penetrating peptides including but not limited to antennapedia, TAT, transportan and positively charged amino acids such as oligoarginine, poly-arginine, oligolysine or polylysine, antigen-binding domains such as provided by an antibody, a Fab fragment, or a single chain antigen binding domain such as a cameloid single domain antigen-binding domain. Accordingly in some embodiments, the ASO is delivered using drug conjugates with antibodies, nanobodies, cell penetrating peptides and aptamers. In one embodiment the oligonucleotide is conjugated to an antibody, preferably a Fab fragment.

The oligonucleotide or composition may be administered as a monotherapy or in combination with a further different medicament, particularly a medicament suitable for the treatment or prevention of alpha-1-antitrypsin (A1AT) deficiency.

Compared to reference oligonucleotides or compositions, the provided oligonucleotides or compositions are surprisingly effective. In some embodiments, a change is measured by an increase of a desired mRNA and/or protein level compared to a reference sample or condition. In some embodiments, a change is measured by an increase in the editing efficacy (%) mediated by the oligonucleotide or composition comprising the same of the invention. In some embodiments, a change is measured by an increase in stability of the oligonucleotide or composition comprising the same. In some embodiments, a change is measured in the levels of cytotoxicity, viability, apoptosis or immune activation. In some embodiments, a change is detected by means of luminescence and/or gene expression. In some embodiments, toxicity and therapeutic efficacy can be determined by standard pharmaceutical procedures in cell cultures or experimental animals, e.g., for determining the LDo (the dose lethal to 50% of the population) and the LD₅₀ (the dose therapeutically effective in 50% of the population). In some embodiments, data obtained from the cell culture assays or animal studies can be used in formulating a range of dosage for use in humans.

Further provided herein is an in vitro method for site directed A-to-I editing of a target RNA, the method comprising a step of contacting a target RNA with the chemically modified oligonucleotide of the invention or the composition of the invention. For instance, the method may be for beneficial and/or compensatory RNA editing. That is, the method may be for targeting wildtype adenosines for beneficial editing or for targeting wildtype adenosines for compensatory editing.

The compositions of the present disclosure can be administered in a number of ways depending upon whether local or systemic treatment is desired and upon the area to be treated. Administration may be by inhalation (e.g., through nebulization), intranasally, orally, by injection or infusion, intravenously, subcutaneously, intra-dermally, intra-cranially, intramuscularly, intra-tracheally, intra-peritoneally, intra-rectally, by direct injection into a tumour, and the like. Administration may be in solid form, in the form of a powder, a pill, or in any other form compatible with pharmaceutical use in humans. In some embodiments the oligonucleotide construct can be delivered systemically.

### Patient Population

The oligonucleotides of the invention of compositions comprising the same may be administered to various groups of subjects or patients. In certain embodiments, the patient is in need of treatment. In other embodiments, the patient is not in need of treatment (“beneficial editing”). That is, the subject receives the oligonucleotide or composition to edit an RNA derived from a wildtype allele (not a mutated allele) in order to modulate the function of the wildtype protein in a useful way.

In one embodiment, an oligonucleotide or composition containing the same is administered to a subject. In some embodiments, an oligonucleotide or composition containing the same is administered to a mammal, preferably a human. In certain embodiments, an oligonucleotide or composition containing the same is administered to a naive subject, i.e., a subject that does not have a disease or disorder. In one embodiment, an oligonucleotide or composition containing the same is administered to a naive subject that is at risk of developing a disease or disorder In some embodiments, an oligonucleotide or composition containing the same is administered to a patient before symptoms manifest or symptoms become severe. In certain embodiments, an oligonucleotide or composition containing the same is administered to a patient who has been diagnosed with a disease or disorder.

In some embodiments, the subject to be administered an oligonucleotide or composition containing the same is any individual at risk of developing a disease or disorder associated with a G-to-A mutation in genes. In one embodiment, the subject suffers from a disease or disorder associated with a G-to-A mutation in genes. In some embodiments, a symptom of a condition, disorder or disease associated with a G-to-A mutation can be any condition, disorder or disease that can benefit from an A-to-I conversion.

Also provided herein are methods of treating a subject suffering from a genetic disease or genetic disorder, comprising administering an effective amount of the chemically modified oligonucleotide of the invention or the composition of the invention. In one embodiment, the genetic disease of genetic disorder is associated with a G-to-A mutation in a subject. Also provided herein is a method for treating a subject suffering from a genetic disease or genetic disorder, comprising administering an effective amount of the chemically modified oligonucleotide of the invention or the composition of the invention. In one embodiment, the genetic disease or disorder is a liver or metabolic diseases and/or cardiac or cardiovascular diseases associated with a gain-of-function (GOF) or loss-of-function (LOF) mutation, optionally wherein the disease or disorder comprises the SERPINA1 gene.

Also provided herein is the use of an oligonucleotide of the invention in therapy. Also, provided herein is a use of an oligonucleotide of the invention in the manufacture of a medicament for treating conditions, diseases and/or disorders associated with a G-to-A mutation. Also provided herein is the use of an oligonucleotide of the invention in the manufacture of a medicament for treating a condition, disease and/or (genetic) disorder associated with a G-to-A mutation. In certain embodiments, the use of an oligonucleotide of the invention is in the manufacture of a medicament for treating a condition, disease and/or disorder associated with a G-to-A mutation

The composition of the invention comprises the oligonucleotide of the invention. According to a further aspect, the invention relates to a kit or kit of parts comprising an oligonucleotide of the invention and/or the (pharmaceutical) composition of the invention. The kit additionally comprises instructions for use.

### Methods for Editing

The present invention also relates to methods for editing a target adenosine in a target nucleic acid. For example, the present invention provides methods of editing a SERPINA1 polynucleotide, e.g., a SERPINA1 polynucleotide comprising a single nucleotide polymorphism (SNP) associated with alpha I antitrypsin deficiency. Alternatively, the target may be human beta actin (hACTB) or a variant thereof. Hence, the invention provides a method for site-directed A-to-I editing of a target RNA, the method comprising a step of contacting a target RNA with the chemically modified oligonucleotide of the invention of the composition of the invention. In one embodiment, the method comprises, after the step of contacting, the following steps: (a) allowing uptake by the cell of the chemically modified oligonucleotide; (b) allowing annealing of the chemically modified oligonucleotide to the target RNA, and (c) allowing a mammalian ADAR enzyme comprising a natural dsRNA binding domain as found in the wild type enzyme to deaminate the target adenosine in the target RNA sequence to an inosine.

The invention also provides an in vitro method for deaminating at least one specific adenosine present in a target RNA sequence in a cell. Hence, in on aspect provided herein is an in vitro method for deaminating at least one specific adenosine present in a target RNA sequence in a cell, wherein the method comprises the steps of: (a) contacting the target nucleic acid with a chemically modified oligonucleotide of the invention; (b) allowing uptake by the cell of the chemically modified oligonucleotide; (c) allowing annealing of the chemically modified oligonucleotide to the target RNA sequence; and (d) allowing a mammalian ADAR enzyme comprising a natural dsRNA binding domain as found in the wild type enzyme to deaminate the target adenosine in the target RNA sequence to an inosine.

In one embodiment, the method comprises after step (d), a step of identifying the presence of the inosine in the RNA sequence.

The editing reaction is preferably monitored or controlled by sequence analysis of the target RNA.

Also, a chemically modified oligonucleotide of the invention or a (pharmaceutical) composition may be used in the diagnosis of a genetic condition, disease or disorder. Therein, the disease or disorder is preferably selected from the group consisting of infectious diseases, tumour diseases, cardiovascular diseases, autoimmune diseases, allergies and neurological diseases or disorders. In one embodiment, the genetic disorder, condition or disease is associated with a G-to-A mutation.

The invention is used to make desired changes in a target sequence in a cell or a subject by site-directed editing of nucleotides using an oligonucleotide that is capable of effecting an adenosine deaminase acting on RNA (ADAR)-mediated adenosine to inosine. As a result, the target sequence is edited through an adenosine deamination reaction mediated by ADAR, converting adenosines Into inosine. In some embodiments, because I is recognized as G, the deamination correcting the pathogenic mutation in the SERPINA1 gene reverses the E342K mutation back to wild-type, reversing or slowing symptoms associated with A1AD experienced by the patient.

The methods of the present invention can be used with cells from any organ, e.g. skin, lung, heart, kidney, liver, pancreas, gut, muscle, gland, eye, brain, blood and the like. The invention is particularly suitable for modifying sequences in cells, tissues or organs implicated in a diseased state of a (human) subject. For example, such cells may include, but are not limited, to hepatocytes, hepatocyte-like cells, and/or alveolar type II cells, neurons (PNS, CNS), retina, photo receptors cells, Müller Glia cells, RPE, immune cells, B cells, T cells, dendritic cells, macrophages.

### EXAMPLES

The present invention shall be described in more detail by the following Examples. The examples shown in the following are merely illustrative and shall describe the present invention in a further way. These examples shall not be construed to limit the present invention thereto. The sequences disclosed herein are also shown in the enclosed sequence listing. However, the sequence listing shows only the sequence of nucleotides, whereas the modification of the nucleotides and of the bonds between the nucleotides is not shown in the sequence listing. The relevant modifications associated with the sequences are disclosed in the tables below and, to some extent, in the Figures of this application.

For all experiments, target editing efficacy is expressed as the percentage [%] of edited target sites found in all detected target sites in the target transcript.

### Example 1

### Modifications Walks Identify Individual Positions Within the Oligonucleotide Beneficial for Target Editing

To determine the effect of different backbone internucleoside linkage modifications, several SERPINA1-specific targeting constructs were generated and tested in vitro and in vivo for their editing efficacy as described below.

Oligonucleotide synthesis: Oligonucleotides were synthesized DMT-ON on a 200 nmol scale using 1000 Å CPG supports from Glen Research; either standard or universal (loading of ca. 30 μmol/g) on a MerMade48 oligonucleotide synthesizer. Fully protected nucleoside phosphoramidites were incorporated using standard solid-phase oligonucleotide synthesis, i.e. 3% dichloroacetic acid in DCM for deblocking, 0.25 M ETT in acetonitrile as activator for amidite couplings, 20% acetic anhydride in THF and 10% 1-methylimidazole in THF/pyridine for capping, 0.02M iodine in THE/water/pyridine for oxidation and 0.1 M xanthane hydride in pyridine; acetonitrile 1:1 (v:v) for thiolation. The mesyl phosphoramidate linkages were obtained via Staudinger reaction, which was carried out with 0.5 M solution of mesyl azide (Aurum Pharmatech) in dry acetonitrile for 15 min at ambient temperature. The guanidine phosphoramidate linkages were also obtained vis Staudinger reaction, from 0.5 M solution of 2-azido-1,3-dimethylimidazolinium hexafluorophosphate (abcr GmbH) in dry acetonitrile for 15 min at ambient temperature. Amidites were dissolved to 0.1 M in acetonitrile and incorporated using 3 min. coupling time for DNA amidites and 6 min. coupling time for all other amidites. After synthesis, oligonucleotides were cleaved from CPG and deprotected at room temperature in 28%-30% ammonium hydroxide and/or 5091/50% mixture of 28% 0% ammonium hydroxide/40% aqueous methylamine (AMA) for 36 hours or 2 h, respectively. Deprotected oligonucleotides were directly adsorbed on GlenPak cartridges and purified DMT-ON. Purified oligonucleotides were dried down, desalted, quantified by means of UV-Vis spectrophotometry and reconstituted in 1×PBS for use in biological experiments Compound Identity was confirmed by LC-MS (Column: DNA-Pac RP; Total flow: 0.5 mL/min.; Oven temperature: 80° C.; Total run time: 10 min.; Eluent gradient: 15-60% B in A; Mobile Phase A: 8 mM Triethylamine (TEA) and 200 mM HFIP in LC-MS grade water; Mobile Phase B: LC-MS grade MeOH).

For triantennary GalNAc coupled oligonucleotides, GalNAc phosphoramidite (e.g. Hongene, cat. no. OP-042) was used for 5′ functionalization of oligonucleotides in an automated fashion. After synthesis and deprotection, oligonucleotides were purified by means of RP-HPLC (Column: Hypersil Gold Semiprep.; Total flow: 3 mL/min.; Oven temperature: 50° C.; Total run time: 40 min.; Eluent gradient: 0-100% B in A; Mobile Phase A: 100 mM phenylboronic acid (PBA) in [10% MeOH/90% 0.2 M aqueous NaOAc]; Mobile Phase B: 100 mM phenylboronic acid (PBA) in [90% MeOH/10% 0.2 M aqueous NaOAc]) and desalted by precipitation with excess of EtOH. After purification quality control and formulation was performed as for oligonucleotides without GalNAc.

Cell culture transfection and mRNA isolation: Hela cells with genomically integrated SERPINA 1 E342K were cultured in DMEM supplemented with 10% FBS (both Gibco) at 37° C. and 5% CO₂ and passaged every 3-4 days Upon 80% confluence, cells were dissociated with Trypsin-ETDA (0.25%) and seeded at 7,500 cells/well in 96-well plates. After 24 hours, cells were transfected with ASOs at indicated final concentration using 0.3 μl Lipofectamine RNAiMAX (Invitrogen) per well in OptiMEM (Gibco).

Transfection: Transfection mix was prepared by mixing equal volumes of 10× concentrated ASO and transfection reagent, and 20 μl of transfection mix was transferred to cells containing 80 μl fresh culture medium. If not stated otherwise, cells were washed with PBS and harvested 24 hours after transfection in 125 μl/well lysis buffer (Dynabeads mRNA direct kit, Invitrogen). Lysates of 96-well plates were transferred to a 384-plate and mRNA was isolated using the Dynabeads mRNA direct kit and an automated plate washer (Cytena C. Wash).

NGS amplicon sequencing: To avoid biases in reverse transcription (RT) mRNA was heated to 90° C. for 2 min with an excess of a sense primer prior to RT. For target amplification of the editing region, a reverse transcription and cDNA amplification was performed with Luna Universal One-Step RT-qPCR mix (NEB) in a 10 μl reaction in a 384-well plate. Both the forward and reverse primer had an overhang to enable a second PCR with primers that bind to that overhang. As presented in Table B, the following primers were used:

TABLE B
SERPINA1 E342K Primer Sequences.

| | | | SEQ |
|---|---|---|---|
| Target | Primer | Sequence | ID NO |
| SERPINA1 | Forward | TCGTCGGCAGCGTCAGATGTGTATAAG | 238 |
| E342K | primer | AGACAGCACCCCTGAAGCTCTCGAAG | |
| SERPINA1 | Reverse | GTCTCGTGGGCTCGGAGATGTGTATAAGA | 238 |
| E342K | primer | GACAGGGATAGACATGGGTATGGCCTC | |
| SERPINA1 | Sense | GCATAAGGCTGTGCTGACCATCGACCCGA | 240 |
| E342K | Primer | AAGGGACTGAAGCTGCTGGGGCCATGAA | |
| SERPINA1 | Forward | TCGTCGGCAGCGTCAGATGTGTATAAGAG | 3 |
| s1 | primer | ACAGGCATCACTAAGGTCTTCAGCAATG | |
| SERPINA1 | Reverse | GTCTCGTGGGCTCGGAGATGTGTATAAGA | 4 |
| s1 | primer | GACAGGGATAGACATGGGTATGGCCTC | |
| SERPINA1 | Sense | AGCTCTCCCCGGCCGTGCATAAGGCTGTG | 5 |
| s1 | Primer | CTGACATA | |

Subsequently, a second POR was performed on the PCR product of the first POR using OneTaq Hot-Start 2×MM with GC buffer (NEB) and forward and reverse primers containing unique indexes as well as adapters for Illumina sequencing. Afterwards, the samples were pooled, and the DNA library was purified with the NucleoSpin Gel and PCR Clean-up Kit (Macherey-Nagel), diluted and sequenced together with a PhiX library on an iSeq 100 (Illumina). Results were analysed using a Python script. Briefly, demultiplexed reads were filtered by quality, length and position before editing percentages were calculated by dividing the number of G reads by the sum of the number of G reads and A reads at the respective target site. Data are represented as mean percentage of editing standard deviation (SD). Replicates are indicated by individual data points in the graphs.

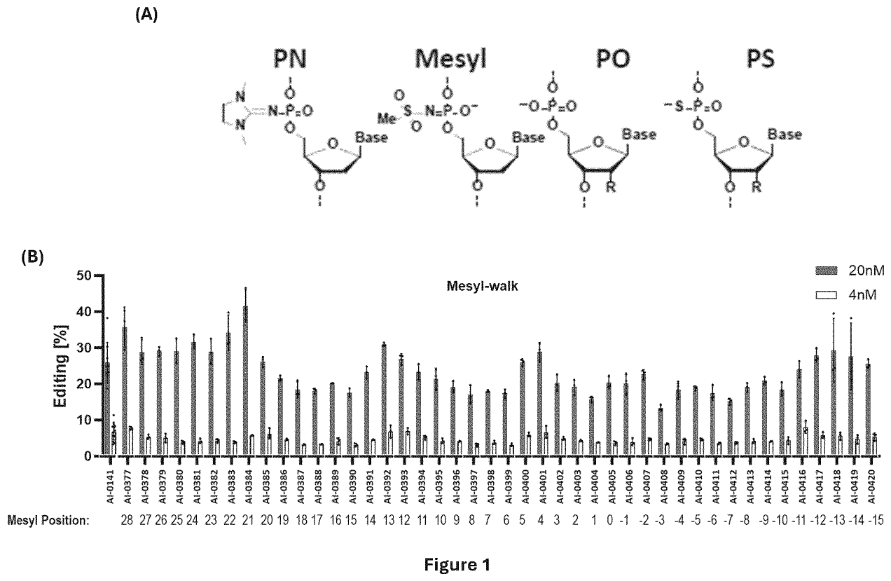

Mesyl linkage modification walk: Oligonucleotides were tested at a concentration of 4 nM and 20 nM. Each ASO candidate differs in the positioning of a single mesyl internucleoside linkage. Different sample oligonucleotides are listed in Table 1A (exemplary sequences of mesyl linkage in a sample construct) and Table 18 (mesyl modification pattern). The results of Example 1 are shown in FIG. 1(B).

TABLE 1A
P-Modifications walks. * = phosphorothioate (PS); & = (mesyl) methanesulfonyl
linkage; # = phosphoryl guanidine (PN); m = 2′-OMe; f = 2′-fluoro; I = Inosine; d = 2′H
(deoxyribose; DNA).

| | | Seq. | Fig. |
|---|---|---|---|
| Construct | Sequence (5′ to 3′ direction) | ID | No. |
| AI-0141 | mC*mA*mU*fG*fG*fC*mC*fC*mC*fA*fG*fC*fA*fG*mC*fU*mU*fC*fA* | 1 | 1B |
| (control; all | fG*mU*fC*mC*fC*mU*mU*mU*fC*dT*dC*dI*mU*fC*fG*fA*mU*fG*fG* | | |
| PS) | fU*mC*fA*fG*mC*mA*mC | | |

Mesyl internucleoside linkage

| | | | |
|---|---|---|---|
| AI-0377 | mC&mA*mU*fG*fG*fC*mC*fC*mC*fA*fG*fC*fA*fG*mC*fU*mU*fC*fA* | 2 | 1B |
| all PS | fG*mU*fC*mC*fC*mU*mU*mU*fC*dT*dC*dI*mU*fC*fG*fA*mU*fG*fG* | | |
| & at 28) | fU*mC*fA*fG*mC*mA*mC | | |

TABLE 1B
Mesyl modifications walks. Summary of the different ASO
constructs generated for the mesyl-walk. The position
of the mesyl linkage is indicated for each construct.

| | |
|---|---|
| | Tye of modification |
| Construct | and position |

Mesyl linkage (&) walks

| | |
|---|---|
| AI-0377 | all PS, & at 28 |
| AI-0378 | all PS, & at 27 |
| AI-0379 | all PS: & at 26 |
| AI-0380 | all PS, & at 25 |
| AI-0381 | all PS, & at 24 |
| AI-0382 | all PS, & at 23 |
| AI-0383 | all PS, & at 22 |
| AI-0384 | all PS, & at 21 |
| AI-0385 | all PS, & at 20 |
| AI-0386 | all PS. & at 19 |
| AI-0387 | all PS, & at 18 |
| AI-0388 | all PS, & at 17 |
| AI-0389 | all PS, & at 16 |
| AI-0380 | all PS, & at 15 |
| AI-0391 | all PS, & at 14 |
| AI-0392 | all PS, & at 13 |
| AI-0393 | all PS, & at 12 |
| AI-0384 | all PS. & at 11 |
| AI-0395 | all PS, & at 10 |
| AI-0386 | all PS, & at 9 |
| AI-0397 | all PS, & at 8 |
| AI-0398 | all PS, & at 7 |
| AI-0399 | all PS, & at 6 |
| AI-0400 | all PS, & at 6 |
| AI-0401 | all PS, & at 4 |
| AI-0402 | all PS, & at 3 |
| AI-0403 | all PS, & at 2 |
| AI-0404 | all PS, & at 1 |
| AI-0405 | all PS, & at 0 |
| AI-0406 | all PS, & at −1 |
| AI-0407 | all PS, & at −2 |
| AI-0408 | all PS, & at −3 |
| AI-0409 | all PS, & at −4 |
| AI-0410 | all PS, & at −5 |
| AI-0411 | all PS, & at −6 |
| AI-0412 | all PS, & at −7 |
| AI-0413 | all PS, & at −8 |
| AI-0414 | all PS, & at −9 |
| AI-0415 | all PS, & at −10 |
| AI-0418 | all PS, & at −11 |
| AI-0417 | all PS, & at −12 |
| AI-0418 | all PS, & at −13 |
| AI-0419 | all PS, & at −14 |
| AI-0420 | all PS, & at −15 |

PS = phosphorothioate linkage; & = mesyl linkage.

The introduction of a single mesyl internucleoside linkage impacted overall in vitro editing (in %) depending on the position of the linkage within the ASO (FIG. 1(B)). For instance, placement of a mesyl internucleoside linkage at, e.g., position +28 to +21, +13, +12, +5, +4, or −11 to −15, resulted in enhanced editing.

Overall, these data show that differential placement of individual internucleoside linkages or linkage modifications at specific positions within the oligonucleotide impacts the overall editing efficacy of the oligonucleotide.

### Example 2

### Identification of Preferred Oligonucleotide Length and Asymmetry for Efficient Editing of SERPINA1 E342K

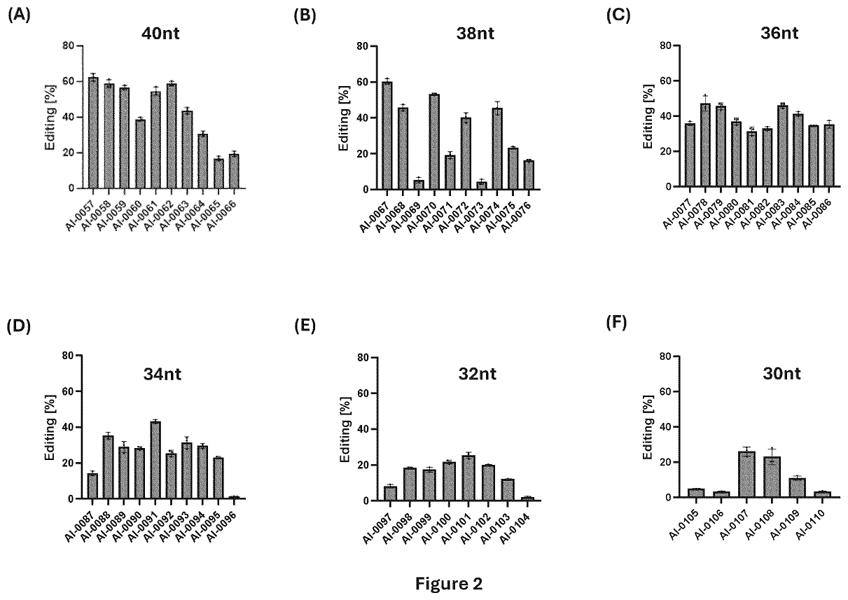

Asymmetry in oligonucleotide sequences can influence their interaction with the target sequences. By introducing asymmetries in the form of varying lengths and sequences, the specificity and efficiency of target binding and editing may be improved. Hence, prior to the introduction of mesyl modifications to the oligonucleotides, it was decided to investigate the combined effect of shorter ASO length and asymmetry, different oligonucleotides were generated and assayed for their in vitro RNA editing efficacy of the SERPINA1 E342K target as described under Example 1 above. Specifically, different SERPINA1-specific oligonucleotide targeting constructs of various lengths, ranging from 30 nt to 38 nt, i.e., 30 nt, 32 nt, 34 nt, 36 nt, or 38 nt, symmetry, and modification pattern were assayed for target editing efficacy (FIG. 2). The different ASO constructs, their asymmetries and modification pattern are listed in Table 2. The ASO constructs have the following structural asymmetry scheme: (length of 5′ terminus)-(1)-(length of 3′ terminus), wherein 1 corresponds to the central nucleotide opposite of the target A. 40 nt long ASOs were used as control. ASOs were tested at a concentration of 25 nM. The results are shown in FIG. 2.

TABLE 2
Candidate oligonucleotides and their asymmetries. * = phosophorothioate (PS);
m = 2′-OMe; f = 2′-fluoro;; I = inosine; d = 2′-H (deoxyribone; DNA).

| | | | Seq. | Fig. |
|---|---|---|---|---|
| Construct | Asymmetry | Sequence (5′ to 3′ direction) | ID | No. |
| AI-0105 | 25-1-4 | mG*mC*mC*fC*mC*fA*fG*fC*fA*fG*mC*fU*mU*fC*fA*fG | 6 | 2 |
| (30 nt) | | *mU*fC*mC*fC*mU*mUmUfC*dT*dC*dImUmCmG | | |
| AI-0106 | 24-1-5 | mC*mC*mC*mC*fA*fG*fC*fA*fG*mC*fU*mU*fC*fA*fG*m | 7 | 2 |
| (30 nt) | | U*fC*mC*fC*mU*mUmUfC*dT*dC*dImUmCmG*mA | | |
| AI-0107 | 23-1-6 | mC*mC*mC*fA*fG*fC*fA*fG*mC*fU*mU*fC*fA*fG*mU*fC | 8 | 2 |
| (30 nt) | | *mC*fC*mU*mUmUfC*dT*dC*dImUfCmG*mA*mU | | |
| AI-0108 | 22-1-7 | mC*mC*mA*fG*fC*fA*fG*mC*fU*mU*fC*fA*fG*mU*fC*m | 9 | 2 |
| (30 nt) | | C*fC*mU*mUmUfC*dT*dC*dImUfCfG*mA*mU*mG | | |
| AI-01089 | 21-1-8 | mC*mA*mG*fC*fA*fG*mC*fU*mU*fC*fA*fG*mU*fC*mC*f | 10 | 2 |
| (30 nt) | | C*mU*mUmUfC*dT*dC*dImUfCfG*mU*mG*mG | | |
| AI-0110 | 20-1-9 | mA*mG*mC*fA*fG*mC*fU*mU*fC*fA*fG*mU*fC*mC*fC* | 11 | 2 |
| (30 nt) | | mU*mUmUfC*dT*dC*dImUfCfG*fA*mU*mG*mG*mU | | |
| AI-0097 | 27-1-4 | mU*mG*mG*fC*mC*fC*mC*fA*fG*fC*fA*fG*mC*fU*mU*f | 12 | 2 |
| (32 nt) | | C*fA*fG*mU*fC*mC*fC*mU*mUmUfC*dT*dC*dImUmCm | | |
| | | G | | |
| AI-0098 | 26-1-5 | mG*mG*mC*mC*fC*mC*fA*fG*fC*fA*fG*mC*fU*mU*fC*f | 13 | 2 |
| (32 nt) | | A*fG*mU*fC*mC*fC*mU*mUmUfC*dT*dC*dImUmCmG* | | |
| | | mA | | |
| AI-009 | 25-1-6 | mG*mC*mC*fC*mC*fA*fG*fC*fA*fG*mC*fU*mU*fC*fA*fG | 14 | 2 |
| (32 nt) | | *mU*fC*mC*fC*mU*mUmUfC*dT*dC*dImUfCmG*mA*m | | |
| | | U | | |
| AI-0100 | 24-1-7 | mC*mC*mC*mC*fA*fG*fC*fA*fG*mC*fU*mU*fC*fA*fG*m | 15 | 2 |
| (32 nt) | | U*fC*mC*fC*mU*mUmUfC*dT*dC*dImUfCfG*mA*mU*m | | |
| | | G | | |
| AI-0101 | 23-1-8 | mC*mC*mC*fA*fG*fC*fA*fG*mC*fU*mU*fC*fA*fG*mU*fC | 16 | 2 |
| (32 nt) | | *mC*fC*mUmUmU*fC*dT*dCdImUfC*fG*fA*mU*mG*mG | | |
| AI-0102 | 22-1-9 | mC*mC*mA*fG*fC*fA*fG*mC*fU*mU*fC*fA*fG*mU*fC*m | 17 | 2 |
| (32 nt) | | C*fC*mU*mUmUfC*dT*dC*dImUfCfG*fA*mU*mG*mG*m | | |
| | | U | | |
| AI-0103 | 21-1-10 | mC*mA*mG*fC*fA*fG*mC*fU*mU*fC*fA*fG*mU*fC*mC*f | 18 | 2 |
| (32 nt) | | C*mU*mUmUfC*dT*dC*dImUfCfG*fA*mU*fG*mG*mU*m | | |
| | | C | | |
| AI-0104 | 20-1-11 | mA*mG*mC*fA*fG*mC*fU*mU*fC*fA*fG*mU*fC*mC*fC* | 19 | 2 |
| (32 nt) | | mU*mUmUfC*dT*dC*dImUfCfG*fA*mU*fG*fG*mU*mC*m | | |
| | | A | | |
| AI-0087 | 29-1-4 | mC*mA*mU*fG*fG*fC*mC*fC*mC*fA*fG*fC*fA*fG*mC*fU | 20 | 2 |
| (34 nt) | | *mU*fC*fA*fG*mU*fC*mC*fC*mU*mUmUfC*dT*dC*dImU | | |
| | | mCmG | | |
| AI-0088 | 28-1-5 | mA*mU*mG*fG*fC*mC*fC*mC*fA*fG*fC*fA*fG*mC*fU*m | 21 | 2 |
| (34 nt) | | U*fC*fA*fG*mU*fC*mC*fC*mU*mUmUfC*dT*dC*dImUm | | |
| | | CmG*mA | | |
| AI-0089 | 27-1-6 | mU*mG*mG*fC*mC*fC*mC*fA*fG*fC*fA*fG*mC*fU*mU*f | 22 | 2 |
| (34 nt) | | C*fA*fG*mU*fC*mC*fC*mU*mUmUfC*dT*dC*dImUfCmG | | |
| | | *mA*mU | | |
| AI-0090 | 26-1-7 | mG*mG*mC*mC*fC*mC*fA*fG*fC*fA*fG*mC*fU*mU*fC*f | 23 | 2 |
| (34 nt) | | A*fG*mU*fC*mC*fC*mU*mUmUfC*dT*dC*dImUfCmG*mA | | |
| | | *mU*mG | | |
| AI-0091 | 25-1-8 | mG*mC*mC*fC*mC*fA*fG*fC*fA*fG*mC*fU*mU*fC*fA*fG | 24 | 2 |
| (34 nt) | | *mU*fC*mC*fC*mU*mUmUfC*dT*dC*dImUfCfG*fA*mU* | | |
| | | mG*mG | | |
| AI-0092 | 24-1-9 | mC*mC*mC*mC*fA*fG*fC*fA*fG*mC*fU*mU*fC*fA*fG*m | 25 | 2 |
| (34 nt) | | U*fC*mC*fC*mU*mUmUfC*dT*dC*dImUfCfG*fA*mU*mG | | |
| | | *mG*mU | | |
| AI-0093 | 23-1-10 | mC*mC*mC*fA*fG*fC*fA*fG*mC*fU*mU*fC*fA*fG*mU*fC | 26 | 2 |
| (34 nt) | | *mC*fC*mU*mUmUfC*dT*dC*dImUfCfG*fA*mU*fG*mG* | | |
| | | mU*mC | | |
| AI-0094 | 22-1-11 | mC*mC*mA*fG*fC*fA*fG*mC*fU*mU*fC*fA*fG*mU*fC*m | 27 | 2 |
| (34 nt) | | C*fC*mU*mUmUfC*dT*dC*dImUfCfG*fA*mU*fG*fG*mU* | | |
| | | mC*mA | | |
| AI-0095 | 21-1-12 | mC*mA*mG*fC*fA*fG*mC*fU*mU*fC*fA*fG*mU*fC*mC*f | 28 | 2 |
| (34 nt) | | C*mU*mUmUfC*dT*dC*dImUfCfG*fA*mU*fG*fG*fU*mC* | | |
| | | mA*mG | | |
| AI-0096 | 20-1-13 | mA*mG*mC*fA*fG*mC*fU*mU*fC*fA*fG*mU*fC*mC*fC* | 29 | 2 |
| (34 nt) | | mU*mUmUfC*dT*dC*dImUfCfG*fA*mU*fG*fG*fU*mC*m | | |
| | | A*mG*mC | | |
| AI-0077 | 29-1-6 | mC*mA*mU*fG*fG*fC*mC*fC*mC*fA*fG*fC*fA*fG*mC*fU | 30 | 2 |
| (34 nt) | | *mU*fC*fA*fG*mU*fC*mC*fC*mU*mUmUfC*dT*dC*dImU | | |
| | | fCmG*mA*mU | | |
| AI-0078 | 28-1-7 | mA*mU*mG*fG*fC*mC*fC*mC*fA*fG*fC*fA*fG*mC*fU*m | 31 | 2 |
| (36 nt) | | U*fC*fA*fG*mU*fC*mC*fC*mU*mUmUfC*dT*dC*dImUfCf | | |
| | | G*mA*mU*mG | | |
| AI0079 | 27-1-8 | mU*mG*mG*fC*mC*fC*mC*fA*fG*fC*fA*fG*mC*fU*mU*f | 32 | 2 |
| (36nt) | | C*fA*fG*mU*fC*mC*fC*mU*mUmUfC*dT*dC*dImUfCfG*f | | |
| | | A*mU*mG*mG | | |
| AI-0080 | 26-1-9 | mG*mG*mC*mC*fC*mC*fA*fG*fC*fA*fG*mC*fU*mU*fC*f | 33 | 2 |
| (36 nt) | | A*fG*mU*fC*mC*fC*mU*mUmUfC*dT*dC*dImUfCfG*fA* | | |
| | | mU*mG*mG*mU | | |
| AI-0081 | 25-1-10 | mG*mC*mC*fC*mC*fA*fG*fC*fA*fG*mC*fU*mU*fC*fA*fG | 34 | 2 |
| (36 nt) | | *mU*fC*mC*fC*mU*mUmUfC*dT*dC*dImUfCfG*fA*mU*f | | |
| | | G*mG*mU*mC | | |
| AI-0082 | 24-1-11 | mC*mC*mC*mC*fA*fG*fC*fA*fG*mC*fU*mU*fC*fA*fG*m | 35 | 2 |
| (36 nt) | | U*fC*mC*fC*mU*mUmUfC*dT*dC*dImUfCfG*fA*mU*fG*f | | |
| | | G*mU*mC*mA | | |
| AI-0083 | 23-1-12 | mC*mC*mC*fA*fG*fC*fA*fG*mC*fU*mU*fC*fA*fG*mU*fC | 36 | 2 |
| (36 nt) | | *mC*fC*mU*mUmUfC*dT*dC*dImUfCfG*fA*mU*fG*fG*fU | | |
| | | *mC*mA*mG | | |
| AI-0084 | 22-1-13 | mC*mC*mA*fG*fC*fA*fG*mC*fU*mU*fC*fA*fG*mU*fC*m | 37 | 2 |
| (36 nt) | | C*fC*mU*mUmUfC*dT*dC*dImUfCfG*fA*mU*fG*fG*fU*m | | |
| | | C*mA*mG*mG*mC | | |
| AI-0085 | 21-1-14 | mC*mA*mG*fC*fA*fG*mC*fU*mU*fC*fA*fG*mU*fC*mC*f | 38 | 2 |
| (36 nt) | | C*mU*mUmUfC*dT*dC*dImUfCfG*fA*mU*fG*fG*fU*mC*f | | |
| | | A*mG*mC*mA | | |
| AI-0086 | 20-1-15 | mA*mG*mC*fA*fG*mC*fU*mU*fC*fA*fG*mU*fC*mC*fC* | 39 | 2 |
| (36 nt) | | mU*mUmUfC*dT*dC*dImUfCfG*fA*mU*fG*fG*fU*mC*fA* | | |
| | | fG*mC*mA*mC | | |
| AI-0067 | 29-1-8 | mC*mA*mU*fG*fG*fC*mC*fC*mC*fA*fG*fC*fA*fG*mC*fU | 40 | 2 |
| (38 nt) | | *mU*fC*fA*fG*mU*fC*mC*fC*mU*mUmUfC*dT*dC*dImU | | |
| | | fCfG*fA*mU*mG*mG | | |
| AI-0068 | 28-1-9 | mA*mU*mG*fG*fC*mC*fC*mC*fA*fG*fC*fA*fG*mC*fU*m | 41 | 2 |
| (38 nt) | | U*fC*fA*fG*mU*fC*mC*fC*mU*mUmUfC*dT*dC*dImUfCf | | |
| | | G*fA*mU*mG*mG*mU | | |
| AI-0069 | 27-1-10 | mU*mG*mG*fC*mC*fC*mC*fA*fG*fC*fA*fG*mC*fU*mU*f | 42 | 2 |
| (38 nt) | | C*fA*fG*mU*fC*mC*fC*mU*mUmUfC*dT*dC*dImUfCfG*f | | |
| | | A*mU*fG*mG*mU*mC | | |
| AI-0070 | 26-1-11 | mG*mG*mC*mC*fC*mC*fA*fG*fC*fA*fG*mC*fU*mU*fC*f | 43 | 2 |
| (38 nt) | | A*fG*mU*fC*mC*fC*mU*mUmUfC*dT*dC*dImUfCfG*fA* | | |
| | | mU*fG*fG*mU*mC*mA | | |
| AI-0071 | 25-1-12 | mG*mC*mC*fC*mC*fA*fG*fC*fA*fG*mC*fU*mU*fC*fA*fG | 44 | 2 |
| (38 nt) | | *mU*fC*mC*fC*mU*mUmUfC*dT*dC*dImUfCfG*fA*mU*f | | |
| | | G*fG*fU*mC*mA*mG | | |
| AI-0072 | 24-1-13 | mC*mC*mC*mC*fA*fG*fC*fA*fG*mC*fU*mU*fC*fA*fG*m | 45 | 2 |
| (38 nt) | | U*fC*mC*fC*mU*mUmUfC*dT*dC*dImUfCfG*fA*mU*fG*f | | |
| | | G*fU*mC*mA*mG*mC | | |
| AI-0073 | 23-1-14 | mC*mC*mC*fA*fG*fC*fA*fG*mC*fU*mU*fC*fA*fG*mU*fC | 46 | 2 |
| (38 nt) | | *mC*fC*mU*mUmUfC*dT*dC*dImUfCfG*fA*mU*fG*fG*fU | | |
| | | *mC*fA*mG*mC*mA | | |
| AI-0074 | 22-1-15 | mC*mC*mA*fG*fC*fA*fG*mC*fU*mU*fC*fA*fG*mU*fC*m | 47 | 2 |
| (38 nt) | | C*fC*mU*mUmUfC*dT*dC*dImUfCfG*fA*mU*fG*fG*fU*m | | |
| | | C*fA*fG*mC*mA*mC | | |
| AI-0075 | 21-1-16 | mC*mA*mG*fC*fA*fG*mC*fU*mU*fC*fA*fG*mU*fC*mC*f | 48 | 2 |
| (38 nt) | | C*mU*mUmUfC*dT*dC*dImUfCfG*fA*mU*fG*fG*fU*mC*f | | |
| | | A*fG*mC*mA*mC*mA | | |
| AI-0076 | 20-1-17 | mA*mG*mC*fA*fG*mC*fU*mU*fC*fA*fG*mU*fC*mC*fC* | 49 | 2 |
| (38 nt) | | mU*mUmUfC*dT*dC*dImUfCfG*fA*mU*fG*fG*fU*mC*fA* | | |
| | | fG*mC*mA*mC*mA*mG | | |
| AI-0057 | 29-1-10 | mC*mA*mU*fG*fG*fC*mC*fC*mC*fA*fG*fC*fA*fG*mC*fU | 50 | 2 |
| (40 nt) | | *mU*fC*fA*fG*mU*fC*mC*fC*mU*mUmUfC*dT*dC*dImU | | |
| | | fCfG*fA*mU*fG*mG*mU*mC | | |
| AI-0058 | 28-1-11 | mA*mU*mG*fG*fC*mC*fC*mC*fA*fG*fC*fA*fG*mC*fU*m | 51 | 2 |
| (40 nt) | | U*fC*fA*fG*mU*fC*mC*fC*mU*mUmUfC*dT*dC*dImUfCf | | |
| | | G*fA*mU*fG*fG*mU*mC*mA | | |
| AI-0059 | 27-1-12 | mU*mG*mG*fC*mC*fC*mC*fA*fG*fC*fA*fG*mC*fU*mU*f | 52 | 2 |
| (40 nt) | | C*fA*fG*mU*fC*mC*fC*mU*mUmUfC*dT*dC*dImUfCfG*f | | |
| | | A*mU*fG*fG*fU*mC*mA*mG | | |
| AI-0060 | 26-1-13 | mG*mG*mC*mC*fC*mC*fA*fG*fC*fA*fG*mC*fU*mU*fC*f | 53 | 2 |
| (40 nt) | | A*fG*mU*fC*mC*fC*mU*mUmUfC*dT*dC*dImUfCfG*fA* | | |
| | | mU*fG*fG*fU*mC*mA*mG*mC | | |
| AI-0061 | 25-1-14 | mG*mC*mC*fC*mC*fA*fG*fC*fA*fG*mC*fU*mU*fC*fA*fG | 54 | 2 |
| (40 nt) | | *mU*fC*mC*fC*mU*mUmUfC*dT*dC*dImUfCfG*fA*mU*f | | |
| | | G*fG*fU*mC*fA*mG*mC*mA | | |
| AI-0062 | 24-1-15 | mC*mC*mC*mC*fA*fG*fC*fA*fG*mC*fU*mU*fC*fA*fG*m | 55 | 2 |
| (40 nt) | | U*fC*mC*fC*mU*mUmUfC*dT*dC*dImUfCfG*fA*mU*fG*f | | |
| | | G*fU*mC*fA*fG*mC*mA*mC | | |
| AI-0063 | 23-1-16 | mC*mC*mC*fA*fG*fC*fA*fG*mC*fU*mU*fC*fA*fG*mU*fC | 56 | 2 |
| (40 nt) | | *mC*fC*mU*mUmUfC*dT*dC*dImUfCfG*fA*mU*fG*fG*fU | | |
| | | *mC*fA*fG*mC*mA*mC*mA | | |
| AI-0064 | 22-1-17 | mC*mC*mA*fG*fC*fA*fG*mC*fU*mU*fC*fA*fG*mU*fC*m | 57 | 2 |
| (40 nt) | | C*fC*mU*mUmUfC*dT*dC*dImUfCfG*fA*mU*fG*fG*fU*m | | |
| | | C*fA*fG*mC*mA*mC*mA*mG | | |
| AI-0065 | 21-1-18 | mC*mA*mG*fC*fA*fG*mC*fU*mU*fC*fA*fG*mU*fC*mC*f | 58 | 2 |
| (40 nt) | | C*mU*mUmUfC*dT*dC*dImUfCfG*fA*mU*fG*fG*fU*mC*f | | |
| | | A*fG*mC*mA*mC*mA*mG*mC | | |
| AI-0066 | 20-1-19 | mA*mG*mC*fA*fG*mC*fU*mU*fC*fA*fG*mU*fC*mC*fC* | 59 | 2 |
| (40 nt) | | mU*mUmUfC*dT*dC*dImUfCfG*fA*mU*fG*fG*fU*mC*fA* | | |
| | | fG*mC*mA*mC*mA*mG*mC*mC | | |

The results show that in shorter ASOs, a unique asymmetry and/or modification pattern may be preferred over another. For instance, as shown in FIG. 2(B) for 38 nt long ASOs, AI-0067 (with an asymmetry of “29-1-8”) showed editing levels of about 60%, which were similar to those of the 40 nt-long control ASO (“29-1-10”) (AI-0057). AI-0070 also showed good editing compared to the longer 40 nt ASOs or other 38 nt-long constructs. Interestingly, there was a strong decrease in editing for AI-0069 (“27-1-10”) and AI-0073 (“23-1-14”). Similar observations were made for the 36 nt long oligonucleotides (FIG. 2(C)). Highest editing levels were obtained for asymmetries of “28-1-7” (AI-0078), “27-1-8” (AI-0079), and “23-1-12” (AI-0083). For the even shorter 34 nt long ASOs, highest editing was observed for a symmetry of “25-1-8” (AI-0091) (FIG. 2(D), while for the 32 nt long constructs an asymmetry of “23-1-8” (AI-0101) showed the highest editing (FIG. 2(E). For the shortest 30 nt long ASOs, highest editing was observed with a “23-1-6” asymmetry (AI-0107) (FIG. 2(F)).

The results show that in shorter oligonucleotides, length and asymmetry of the ASO have a significant combined effect on SERPINA 1 E342K target editing. Based on the results presented in FIG. 2, it may be preferable to choose specific asymmetries depending on the length of the oligonucleotide, e.g., “29-1-8” (38 nt), “23-1-12” (36 nt), “26-1-8” and “27-1-6” (34 nt), “23-1-8” (32 nt) and “26-1- 6” (30 nt).

### Example 3

### Combination of High 2′-F Modifications With Nucleotide and Internal Internucleoside Linkage Modifications or in the 5′ and 3′ Flanking Regions Increases SERPINA1 Target Editing

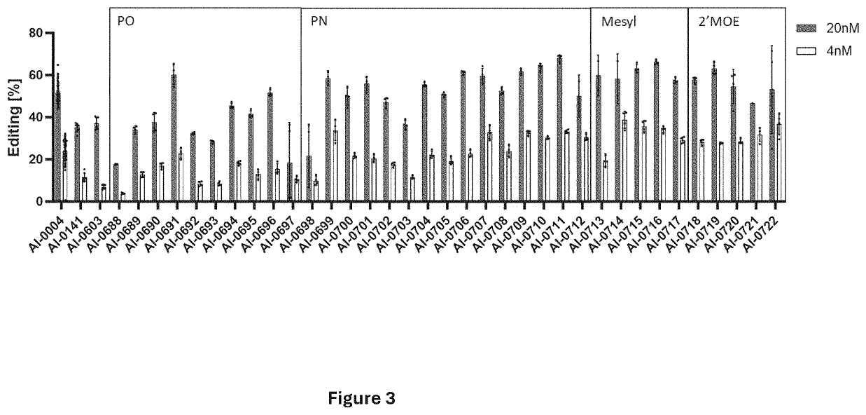

To determine whether high levels of 2′-F modifications in combination with other internucleoside linkage and 2′-sugar modifications could enhance editing efficacy in shorter ASOs of varying asymmetries. SERPINA1-specific ASO constructs of 30nt (“23-1-6” asymmetry) to 38nt (“29-1-8” asymmetry) were generated as described above. These constructs comprise different permutations of high levels of 2′-F modifications, PO, PN or mesyl linkages, or 2′-MOE modifications. Editing efficacy was assessed as described in Example 1. The different oligonucleotide constructs and their respective modifications and sequences are listed in Table 3. The various shorter ASO constructs were assessed for editing efficacy (in %) and compared to two 45nt-long ASOs (“29-1-15” asymmetry; partial PS (AI-0004) or fully modified PS backbone (AI-0141) and a 30 nt long ASO (“23-1-6” asymmetry, VW-44464 w/o stereopure, 8-OxoA, AI-0603). The different oligonucleotide candidates were tested at concentrations of 4 nM and 20 nM. The results are presented in FIG. 3.

TABLE 3
Oligonucleotide constructs and modifications. * = phosphorothioate (PS); PO =
phospate; # = phosphoryl guanidine (PN); m = 2′-OMe; f = 2′-fluoro; d = 2′-H
(deoxyribose; DNA); i = inosine; moe = 2′-O-Methoxyethyl (2′-MOE); & = (mesyl)
methanesulfonyl; MeC = 5′-methylcytidine.

| Construct | | Seq. | Fig. |
|---|---|---|---|
| (Asymmetry) | Modification Pattern and Sequence (5′-3′ direction) | ID | No. |
| AI-0004 | mC*mA*mU*fG*fG*fC*mC*fC*mC*fA*fG*fC*fA*fG*mC*fU*mU*fC*fA* | 60 | 3 |
| (29-1-15) | fG*mU*fC*mC*fC*mU*mUmUfC*dT*dC*dimUfCfG*fA*mU*fG*fG*fU* | | |
| | mC*fA*fG*mC*mA*mC | | |
| AI-0141 | (all PS) | 1 | 3 |
| (29-1-15) | mC*mA*mU*fG*fG*fC*mC*fC*mC*fA*fG*fC*fA*fG*mC*fU*mU*fC*fA* | | |
| | fG*mU*fC*mC*fC*mU*mU*mU*fC*dT*dC*di*mU*fC*fG*fA*mU*fG*f | | |
| | G*fU*mC*fA*fG*mC*mA*mC | | |
| AI-0603 | mC#mC*mC*fA*fG*mC*fA*fG*fC*fU*fU#fC*fAfG#fU*fC*fC*fC*fU*mU | 61 | 3 |
| (23-1-6) | fU*fC*fU*dC*di#mUfC*mGmA#mU | | |

PO Linkages

| | | | |
|---|---|---|---|
| AI-0688 | (17F PO 3, −2, −3, −4) | 62 | 3 |
| (23-1-6) | mC*mC*fC*mA*fG*mC*fA*fG*fC*fU*fU*mC*fA*mG*fU*fC*fC*fC*fU* | | |
| | mUfU*fC*fU*dC*dimUfCmG*mA*mU | | |
| AI-0689 | (18F PO 3, −2, −3, −4) | 63 | 3 |
| (25-1-8) | mG*mC*mC*fC*fC*mA*fG*mC*fA*fG*fC*fU*fU*mC*fA*mG*fU*fC*fC | | |
| | *fC*fU*mUfU*fC*fU*dC*dimUfCmG*mA*mU*mG*mG | | |
| AI-0690 | (20F PO 3, −2, −3, −4) | 64 | 3 |
| (27-1-5) | mU*mG*mG*fC*fC*fC*fC*mA*fG*mC*fA*fG*fC*fU*fU*mC*fA*mG*fU | | |
| | *fC*fC*fC*fU*mUfU*fC*fU*dC*dimUfCmG*mA*mU | | |
| AI-0691 | (21F PO 3, −2, −3, −4) | 65 | 3 |
| (29-1-8) | mC*mA*mU*mG*fG*fC*fC*fC*fC*mA*fG*mC*fA*fG*fC*fU*fU*mC*fA* | | |
| | mG*fU*fC*fC*fC*fU*mUfU*fC*fU*dC*dimUfCmG*mA*mU*mG*mG | | |
| AI-0692 | (19F PO 3, −2, −3, −4) | 66 | 3 |
| (23-1-12) | mC*mC*fC*mA*fG*mC*fA*fG*fC*fU*fU*mC*fA*mG*fU*fC*fC*fC*fU* | | |
| | mUfU*fC*fU*dC*dimUfCmG*mA*fU*mG*fG*mU*mC*mA*mG | | |
| AI-0693 | (17F PO 3, −2, −3) | 67 | 3 |
| (23-1-6) | mC*mC*fC*mA*fG*mC*fA*fG*fC*fU*fU*mC*fA*mG*fU*fC*fC*fC*fU* | | |
| | MufU*fC*fU*dC*dimUfC*mG*mA*mU | | |
| AI-0694 | (18F PO 3, −2, −3) | 68 | 3 |
| (25-1-8) | mG*mC*mC*fC*fC*mA*fG*mC*fA*fG*fC*fU*fU*mC*fA*mG*fU*fC*fC | | |
| | *fC*fU*mUfU*fC*fU*dC*dimUfC*mG*mA*mU*mG*mG | | |
| AI-0695 | (20F PO 3, −2, −3) | 69 | 3 |
| (27-1-6) | mU*mG*mG*fC*fC*fC*fC*mA*fG*mC*fA*fG*fC*fU*fU*mC*fA*mG*fU | | |
| | *fC*fC*fC*fU*mUfU*fC*fU*dC*dimUfC*mG*mA*mU | | |
| AI-0696 | (21F PO 3, −2, −3) | 70 | 3 |
| (29-1-8) | mC*mA*mU*mG*fG*fC*fC*fC*fC*mA*fG*mC*fA*fG*fC*fU*fU*mC*fA* | | |
| | mG*fU*fC*fC*fC*fU*mUfU*fC*fU*dC*dimUfC*mG*mA*mU*mG*mG | | |
| AI-0697 | (19F PO 3, −2, −3) | 71 | 3 |
| (23-1-12) | mC*mC*fC*mA*fG*mC*fA*fG*fC*fU*fU*mC*fA*mG*fU*fC*fC*fC*fU* | | |
| | mUfU*fC*fU*dC*dimUfC*mG*mA*fU*mG*fG*mU*mC*mA*mG | | |

PN Linkage Modification

| | | | |
|---|---|---|---|
| AI-0698 | (17F PO 3, −3, PN −2) | 72 | 3 |
| (23-1-5) | mC*mC*fC*mA*fG*mC*fA*fG*fC*fU*fU*mC*fA*mG*fU*fC*fC*fC*fU* | | |
| | mUfU*fC*fU*dC*di#mUfC*mG*mA*mU | | |
| AI-0699 | (18F PO 3, −3, PN −2) | 73 | 3 |
| (25-1-8) | mG*mC*mC*fC*fC*mA*fG*mC*fA*fG*fC*fU*fU*mC*fA*mG*fU*fC*fC | | |
| | *fC*fU*mUfU*fC*fU*dC*di#mUfC*mG*mA*mU*mG*mG | | |
| AI-0700 | (20F PO 3, −3, PN −2) | 74 | 3 |
| (27-1-6) | mU*mG*mG*fC*fC*fC*fC*mA*fG*mC*fA*fG*fC*fU*fU*mC*fA*mG*fU | | |
| | *fC*fC*fC*fU*mUfU*fC*fU*dC*di#mUfC*mG*mA*mU | | |
| AI-0701 | (21F PO 3, −3, PN −2) | 75 | 3 |
| (29-1-8) | mC*mA*mU*mG*fG*fC*fC*fC*fC*mA*fG*mC*fA*fG*fC*fU*fU*mC*fA* | | |
| | mG*fU*fC*fC*fC*fU*mUfU*fC*fU*dC*di#mUfC*mG*mA*mU*mG*mG | | |
| AI-0702 | (19F PO 3, −3, PN −2) | 76 | 3 |
| (23-1-12) | mC*mC*fC*mA*fG*mC*fA*fG*fC*fU*fU*mC*fA*mG*fU*fC*fC*fC*fU* | | |
| | mUfU*fC*fU*dC*di#mUfC*mG*mA*fU*mG*fG*mU*mC*mA*mG | | |
| AI-0703 | (17F PO 3, −3, PN −2, −5) | 77 | 3 |
| (23-1-6) | mC*mC*fC*mA*fG*mC*fA*fG*fC*fU*fU*mC*fA*mG*fU*fC*fC*fC*fU* | | |
| | mUfU*fC*fU*dC*di#mUfC*mG#mA*mU | | |
| AI-0704 | (1 18F PO 3, −3, PN −2, −5) | 78 | 3 |
| (25-1-8) | mG*mC*mC*fC*fC*mA*fG*mC*fA*fG*fC*fU*fU*mC*fA*mG*fU*fC*fC | | |
| | *fC*fU*mUfU*fC*fU*dC*di#mUfC*mG#mA*mU*mG*mG | | |
| AI-0705 | (20F PO 3, −3, PN −2, −5) | 79 | 3 |
| (27-1-6) | mU*mG*mG*fC*fC*fC*fC*mA*fG*mC*fA*fG*fC*fU*fU*mC*fA*mG*fU | | |
| | *fC*fC*fC*fU*mUfU*fC*fU*dC*di#mUfC*mG#mA*mU | | |
| AI-0706 | (21F PO 3, −3, PN −2, −5) | 80 | 3 |
| (29-1-8) | mC*mA*mU*mG*fG*fC*fC*fC*fC*mA*fG*mC*fA*fG*fC*fU*fU*mC*fA* | | |
| | mG*fU*fC*fC*fC*fU*mUfU*fC*fU*dC*di#mUfC*mG#mA*mU*mG*mG | | |
| AI-0707 | (19F PO 3, −3, PN −2, −5) | 81 | 3 |
| (23-1-2) | mC*mC*fC*mA*fG*mC*fA*fG*fC*fU*fU*mC*fA*mG*fU*fC*fC*fC*fU* | | |
| | mUfU*fC*fU*dC*di#mUfC*mG#mA*fU*mG*fG*mU*mC*mA*mG | | |
| AI-0708 | (17F PO 3, −3, PN 22, −6) | 82 | 3 |
| (23-1-6) | mC*#mC*fC*mA*fG*mC*fA*fG*fC*fU*fU*mC*fA*mG*fU*fC*fC*fC*fU* | | |
| | mUfU*fC*fU*dC*di#mUfC*mG*mA#mU | | |
| AI-0709 | (18F PO 3, −3, PN 24, −6) | 83 | 3 |
| (25-1-8) | mG#*mC*mC*fC*fC*mA*fG*mC*fA*fG*fC*fU*fU*mC*fA*mG*fU*fC*fC | | |
| | *fC*fU*mUfU*fC*fU*dC*di#mUfC*mG*mA*mU*mG#mG | | |
| AI-0710 | (20F PO 3, −3, PN 26, −2, −6) | 84 | 3 |
| (27-1-6) | mU#mG*mG*fC*fC*fC*fC*mA*fG*mC*fA*fG*fC*fU*fU*mC*fA*mG*fU | | |
| | *fC*fC*fC*fU*mUfU*fC*fU*dC*di#mUfC*mG*mA#mU | | |
| AI-0711 | (21F PO 3, −3, PN 28, −2, −8) | 85 | 3 |
| (29-1-8) | mC#mA*mU*mG*fG*fC*fC*fC*fC*mA*fG*mC*fA*fG*fC*fU*fU*mC*fA | | |
| | *mG*fU*fC*fC*fC*fU*mUfU*fC*fU*dC*di#mUfC*mG*mA*mU*mG#m | | |
| | G | | |
| AI-0712 | (19F PO 3, −3, PN 22, −2, −12) | 86 | 3 |
| (23-1-12) | mC#mC*fC*mA*fG*mC*fA*fG*fC*fU*fU*mC*fA*mG*fU*fC*fC*fC*fU* | | |
| | mUfU*fC*fU*dC*di#mUfC*mG*mA*fU*mG*fG*mU*mC*mA#mG | | |

Mesyl Linkage Modification

| | | | |
|---|---|---|---|
| AI-0713 | (17F PO 3, −3, PN −2, Mesyl 22, −6) | 87 | 3 |
| (23-1-6) | mC&mC*fC*mA*fG*mC*fA*fG*fC*fU*fU*mC*fA*mG*fU*fC*fC*fC*fU* | | |
| | mUfU*fC*fU*dC*di#mUfC*mG*mA&mU | | |
| AI-0714 | (18F PO 3, −3, PN −2, Mesyl 24, −8) | 88 | 3 |
| (25-1-8) | mG&mC*mC*fC*fC*mA*fG*mC*fA*fG*fC*fU*fU*mC*fA*mG*fU*fC*fC | | |
| | *fC*fU*mUfU*fC*fU*dC*di#mUfC*mG*mA*mU*mG&mG | | |
| AI-0715 | (20F PO 3, −3, PN −2, Mesyl 26, −6) | 89 | 3 |
| (27-1-6) | mU&mG*mG*fC*fC*fC*fC*mA*fG*mC*fA*fG*fC*fU*fU*mC*fA*mG*fU | | |
| | *fC*fC*fC*fU*mUfU*fC*fU*dC*di#mUfC*mG*mA&mU | | |
| AI-0716 | (21F PO 3, −3, PN −2, Mesyl 28, −8) | 90 | 3 |
| (29-1-8) | mC&mU*mG*fG*fC*fC*fC*fC*mA*fG*mC*fA*fG*fC*fU*fU*mC*fA | | |
| | *mG*fU*fC*fC*fC*fU*mUfU*fC*fU*dC*di#mUfC*mG*mA*mU*mG&m | | |
| | G | | |
| AI-0717 | (19F PO 3, −3, PN −2, Mesyl 22, −12) | 91 | 3 |
| (23-1-12) | mC&mC*fC*mA*fG*mC*fA*fG*fC*fU*fU*mC*fA*mG*fU*fC*fC*fC*fU* | | |
| | mUfU*fC*fU*dC*di#mUfC*mG*mA*fU*mG*fG*mU*mC*mA&mG | | |

2′MOE Modifications

| | | | |
|---|---|---|---|
| AI-0718 | (17F PO 3, −3, PN −2, MOE 23, 22, 1, −4, −5, −6) | 92 | 3 |
| (23-1-6) | moe(MeC)*moe(MeC)*fC*mA*fG*mC*fA*fG*fC*fU*fU*mC*fA*mG*fU | | |
| | *fC*fC*fC*fU*mUfU*fC*moeT*dC*di#mUfC*moeG*moeA*moeT | | |
| AI-0719 | (18F PO 3, −3, PN −2, MOE 25, 24, 23, 1, −4, −6, −7, −8) | 93 | 3 |
| (25-1-8) | moeG*moe(MeC)*moe(MeC)*fC*fC*mA*fG*mC*fA*fG*fC*fU*fU*mC* | | |
| | fA*mG*fU*fC*fC*fC*fU*mUfU*fC*moeT*dC*di#mUfC*moeG*mA*mo | | |
| | eT*moeG*moeG | | |
| AI-0720 | (20F PO 3, −3, PN −2, MOE 27, 26, 25, 1, −4, −5, −6) | 94 | 3 |
| (27-1-6) | moeT*moeG*moeG*fC*fC*fC*fC*mA*fG*mC*fA*fG*fC*fU*fU*mC*fA* | | |
| | mG*fU*fC*fC*fC*fU*mUfU*fC*moeT*dC*di#mUfC*moeG*moeA*moe | | |
| | T | | |
| AI-0721 | (21F PO 3, −3, PN −2, MOE 29, 28, 27, 1, −4, −6, −7, −8) | 95 | 3 |
| (29-1-8) | moe(MeC)*moeA*moeT*mG*fG*fC*fC*fC*fC*mA*fG*mC*fA*fG*fC*f | | |
| | U*fU*mC*fA*mG*fU*fC*fC*fC*fU*mUfU*fC*moeT*dC*di#mUfC*moe | | |
| | G*mA*moeT*moeG*moeG | | |
| AI-0722 | (19F PO 3, −3, PN −2, MOE 23, 22, 1, −4, −10, −11, −12) | 96 | 3 |
| (23-1-12) | moe(MeC)*moe(MeC)*fC*mA*fG*mC*fA*fG*fC*fU*fU*mC*fA*mG*fU | | |
| | *fC*fC*fC*fU*mUfU*fC*moeT*dC*di#mUfC*moeG*mA*fU*mG*fG*m | | |
| | U*moe(MeC)*moeA*moeG | | |

Generally, It was shown that introducing mesyl, PN and/or 2′-MOE enhanced editing in shorter ASOs (FIG. 3). ASOs carrying a mixture of 2′F-sugar modifications and PO linkages (AI-0688 to AI-0697) showed editing that was generally lower than that of ASOs carrying mesyl, PN or 2′OME modifications. Furthermore, shorter ASOs did perform worse in the group of ASOs only containing 2′F and PO (e.g., AI-0688 (30 nt) vs AI-0690 (34 nt). This trend was also observed at 4 nM. Addition and/or replacement of PO linkages by PN linkages led to an Increase in editing efficacy by the shorter 30 nt long ASO, e.g., AI-0688 vs AI-0703. A similar trend was observed for 34 nt long ASOs (e.g., AI-0689 vs AI-0699) and 36 nt long ASOs (e.g., AI-0692 vs AI-0702). Modifications of shorter ASOs generally led to an increase in editing efficacy that was similar to that of the 45 nt long control (AI-0004).

Furthermore, introducing mesyl linkages into the ASO tended to increase editing, especially at the lower concentration of 4 nM. For instance, as shown with the “PN-2, mesyl 22, −6” modified oligonucleotide AI-0713 (30 nt), having mesyl modified linkages at the outmost 5′ and 3′ flanking positions +22 and −6, not only increased editing efficacy when compared to the “PN-2” ASO (AI-0698) but also when compared to the 45 nt long control ASO.

The results show that the positioning of 2″-F modifications impacts on the editing efficacy of the ASO. Furthermore, the results show that PN (constructs AI-0708 to AI-0712) and mesyl (constructs AI-0713 to AI-0717) internucleoside linkages as well as 2′-MOE modifications (constructs AI-0718 to AI-0722) in the 5 and 3′ regions flanking the base that is to be edited increase the editing efficiency of short ASOs. In particular, shortened constructs AI-0713 to AI-0717, which contain mesyl linkages at the 5′ and 3′ terminal ends showed good editing similar to the 45 nt long ASO. The inventors further showed that 2′-MOE modifications could be placed 5′ and 3′ of the CBT and/or at the termini of the oligonucleotides without reducing the overall editing of the ASO.

Notably, to determine whether the types and number of modifications supports the editing efficacy of shorter ASOs even at lower concentrations, the different candidates were tested for their editing efficacy at a concentration of 4 nM As shown in FIG. 3, the inventors found that the positioning of mesyl modifications within the 3′-and 5′-regions of each ASO increased overall editing, not only at 20 nM but also at the lower concentration of 4 nM. These results show that ASOs comprising internucleoside linkage modifications according to the invention show increased editing even at lower concentrations.

Importantly, the results show that at 20 nM and even 4 nM the different test constructs have greater editing efficacy than ASOs known in the art at (e.g., AI-0603). Overall, the results show that combining 2′-F modifications with other 2′-sugar modifications (e.g., 2′-OMe or 2′MOE) and mesyl internucleoside linkages in flanking regions increases editing efficacy above that of the prior art oligonucleotides.

### Example 4

### Optimised Combinations of 2′-Sugar Modifications and Internucleoside Linkages Increase SERPINA1 Target Editing

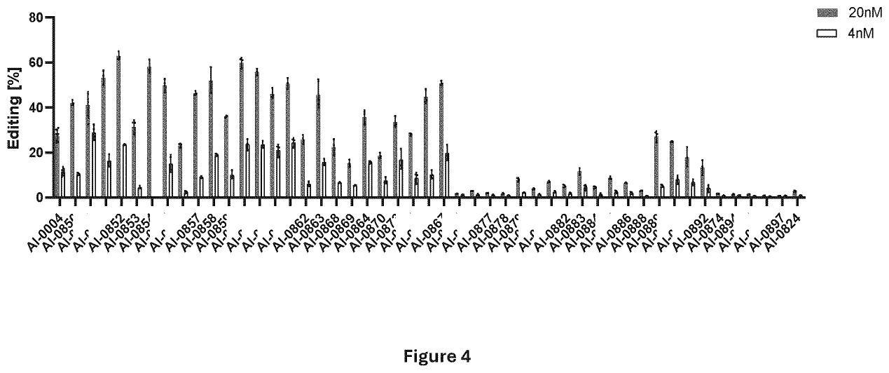

To determine whether the mesyl internucleoside linkage content has an impact on the editing efficacy (in %) of different ASOs, oligonucleotides were synthesised and assayed as described above. The test constructs differ in their internucleoside linkage (e.g., mesyl, PS and PO) and 2′-sugar modification (e.g., 2′-OMe, 2′-F and/or 2′-MOE) content. For instance, ASOs were generated comprising a higher mesyl content, white expressing lower 2′-F modifications and PS linkages. The different oligonucleotide constructs and their respective modifications and sequence are listed in Table 4. The different oligonucleotide candidates were tested at concentrations of 4 nM and 20 nM. The results are shown in FIG. 4.

TABLE 4
Oligonucleotide constructs and modifications. * = phosphorothioate (PS);
m = 2′-OMe; & = (mesyl) methanesulfonyl; d = 2′-H (deoxyribose; DNA);
phospate; # = phosphoryl guanidine (PN); f = 2′-fluoro; i = inosine; f or F = 2′-
fluoro; moe = 2′-O-Methoxyethyl (2′-MOE); # = phosphoryl guanidine (PN); PO = phosphate.

| Construct | | Seq. | Fig. |
|---|---|---|---|
| (Asymmetry) | Modification Pattern and Sequence (5′-3′ direction) | ID NO | No. |
| AI-0004 | mC*mA*mU*fG*fG*fC*mC*fC*mC*fA*fG*fC*fA*fG*mC*fU*mU*fC* | 60 | 4 |
| (29-1-15) | fA*fG*mU*fC*mC*fC*mU*mUmUfC*dT*dC*dimUfCfG*fA*mU*fG*f | | |
| | G*fU*mC*fA*fG*mC*mA*mC | | |
| AI-0850 | (17F PO 3, −3, PN −2 Mesyl 22, 13, 4, −6) | 97 | 4 |
| (23-1-6) | mC&mC*fC*mA*fG*mC*fA*fG*fC*fU&fU*mC*fA*mG*fU*fC*fC*fC* | | |
| | fU&mUfU*fC*fU*dC*di#mUfC*mG*mA&mU | | |
| AI-0714 | (18F PO 3, −3, PN −2 Mesyl 24, −8) | 86 | 4 |
| (25-1-8) | mG&mC*mC*fC*fC*mA*fG*mC*fA*fG*fC*fU*fU*mC*fA*mG*fU*fC | | |
| | *fC*fC*fU*mUfU*fC*fU*dC*di#mUfC*mG*mA*mU*mG&mG | | |
| AI-0851 | (18F PO 3, −3, PN −2 Mesyl 24, 21, 13, 4, −6) | 98 | 4 |
| (25-1-8) | mG&mC*mC*fC&fC*mA*fG*mC*fA*fG*fC*fU&fU*mC*fA*mG*fU*f | | |
| | C*fC*fC*fU&mUfU*fC*fU*dC*di#mUfC*mG*mA*mU*mG&mG | | |
| AI-0852 | (19F PO 3, −3, PN −2 Mesyl 22, 13, 4, −12) | 99 | 4 |
| (23-1-12) | mC&mC*fC*mA*fG*mC*fA*fG*fC*fU&fU*mC*fA*mG*fU*fC*fC*fC* | | |
| | fU&mUfU*fC*fU*dC*di#mUfC*mG*mA*fU*mG*fG*mU*mC*mA& | | |
| | mG | | |
| AI-0853 | (17F PO 17, 10, 3, −3, PN −2 Mesyl 22, 13, 4, −6) | 100 | 4 |
| (23-1-6) | mC&mC*fC*mA*fG*mCfA*fG*fC*fU&fU*mC*fAmG*fU*fC*fC*fC*f | | |
| | U&mUfU*fC*fU*dC*di#mUfC*mG*mA&mU | | |
| AI-0854 | (18F PO 17, 10, 3, −3, PN −2 Mesyl 24, 21, 13, 4, −8) | 101 | 4 |
| (25-1-8) | mG&mC*mC*fC&fC*mA*fG*mCfA*fG*fC*fU&fU*mC*fAmG*fU*fC | | |
| | *fC*fC*fU&mUfU*fC*fU*dC*di#mUfC*mG*mA*mU*mG&mG | | |
| AI-0855 | (19F PO 17, 10, 3, −3, PN −2 Mesyl 22, 13, 4, −12) | 102 | 4 |
| (23-1-12) | mC&mC*fC*mA*fG*mCfA*fG*fC*fU&fU*mC*fAmG*fU*fC*fC*fC*f | | |
| | U&mUfU*fC*fU*dC*di#mUfC*mG*mA*mU*mG*fG*mU*mC*mA&m | | |
| | G | | |
| AI-0856 | (17F PO 14, 6, 3, −3, PN −2 Mesyl 22, 13, 4, −6) | 103 | 4 |
| (23-1-6) | mC&mC*fC*mA*fG*mCfA*fG*fCfU&fU*mC*fAmG*fU*fC*fCfC*fU | | |
| | &mUfU*fC*fU*dC*di#mUfC*mG*mA&mU | | |
| AI-0857 | (18F PO 23, 14, 6, 3, −3, PN −2 Mesyl 24, 21, 13, 4, −8) | 104 | 4 |
| (25-1-8) | mG&mCmC*fC&fC*mA*fG*mCfA*fG*fCfU&fU*mC*fAmG*fU*fC*f | | |
| | CfC*fU&mUfU*fC*fU*dC*di#mUfC*mG*mA*mU*mG&mG | | |
| AI-0858 | (19F PO 14, 6, 3, −3, −10 PN −2 Mesyl 22, 13, 4, −12) | 105 | 4 |
| (23-1-12) | mC&mC*fC*mA*fG*mCfA*fG*fCfU&fU*mC*fAmG*fU*fC*fCfC*fU | | |
| | &mUfU*fC*fU*dC*di#mUfC*mG*mA*fU*mG*fG*mUmC*mA&mG | | |
| AI-0859 | (17F PO 3, −3, Mesyl 22, 13, 4, −2, −6) | 106 | 4 |
| (23-1-6) | mC&mC*fC*mA*fG*mC*fA*fG*fC*fU&fU*mC*fA*mG*fU*fC*fC*fC* | | |
| | fU&mUfU*fC*fU*dC*di#mUfC*mG*mA&mU | | |
| AI-0860 | (18F PO 3, −3, Mesyl 24, 21, 13, 4, −2, −8) | 107 | 4 |
| (25-1-8) | mG&mC*mC*fC&fC*mA*fG*mC*fA*fG*fC*fU&fU*mC*fA*mG*fU*f | | |
| | C*fC*fC*fU&mUfU*fC*fU*dC*di#mUfC*mG*mA*mU*mG&mG | | |
| AI-0861 | (19F PO 3, −3, Mesyl 22, 13, 4, −2, −12) | 108 | 4 |
| (23-1-12) | mC&mC*fC*mA*fG*mC*fA*fG*fC*fU&fU*mC*fA*mG*fU*fC*fC*fC* | | |
| | fU&mUfU*fC*fU*dC*di#mUfC*mG*mA*fU*mG*fG*mU*mC*mA& | | |
| | mG | | |
| AI-0871 | (18F, Mesyl 24, −2, −6) | 109 | 4 |
| (23-1-12) | mG&mC*mC*fC*fC*mA*fG*mC*fA*fG*fC*fU*fU*mC*fA*mG*fU*fC | | |
| | *fC*fC*fU*mU*fU*fC*fU*dC*di#mU*fC*mG*mA*mU*mG&mG | | |
| AI-0872 | (18F, PO 3, −3, Mesyl 24, −2, −8) | 110 | 4 |
| (25-1-8) | mG&mC*mC*fC*fC*mA*fG*mC*fA*fG*fC*fU*fU*mC*fA*mG*fU*fC | | |
| | *fC*fC*fU*mUfU*fC*fU*dC*di#mUfC*mG*mA*mU*mG&mG | | |
| AI-0862 | (17F PN −2, Mesyl 22, 13, −4, −6) | 111 | 4 |
| (23-1-6) | mC&mC*fC*mA*fG*mC*fA*fG*fC*fU&fU*mC*fA*mG*fU*fC*fC*fC* | | |
| | fU&mU*fU*fC*fU*dC*di#mU*fC*mG*mA&mU | | |
| AI-0863 | (18F, PN −2, Mesyl 24, 21, 13, −4, −6) | 112 | 4 |
| (25-1-8) | mG&mC*mC*fC&fC*mA*fG*mC*fA*fG*fC*fU&fU*mC*fA*mG*fU*f | | |
| | C*fC*fC*fU&mU*fU*fC*fU*dC*di#mU*fC*mG*mA*mU*mG&mG | | |
| AI-0868 | (18F, PN −2, Mesyl 24, 21, 13, 3, −3, −8) | 113 | 4 |
| (25-1-8) | mG&mC*mC*fC&fC*mA*fG*mC*fA*fG*fC*fU&fU*mC*fA*mG*fU*f | | |
| | C*fC*fC*fU*mU&fU*fC*fU*dC*di#mU&fC*mG*mA*mU*mG&mG | | |
| AI-0869 | (18F, PN −2, Mesyl 24, 3, −3, −8) | 114 | 4 |
| (25-1-8) | mG&mC*mC*fC*fC*mA*fG*mC*fA*fG*fC*fU*fU*mC*fA*mG*fU*fC | | |
| | *fC*fC*fU*mU&fU*fC*fU*dC*di#mU&fC*mG*mA*mU*mG&mG | | |
| AI-0864 | (19F, PN −2, Mesyl 24, 13, 4, −12) | 115 | 4 |
| (23-1-12) | mC&mC*fC*mA*fG*mC*fA*fG*fC*fU&fU*mC*fA*mG*fU*fC*fC*fC* | | |
| | fU&mU*fU*fC*fU*dC*di#mU*fC*mG*mA*fU*mG*fG*mU*mC*mA& | | |
| | mG | | |
| AI-0870 | (18F, Mesyl 24, 3, −2, −3, −8) | 116 | 4 |
| (25-1-8) | mG&mC*mC*fC*fC*mA*fG*mC*fA*fG*fC*fU*fU*mC*fA*mG*fU*fC | | |
| | *fC*fC*fU*mU&fU*fC*fU*dC*di#mU&fC*mG*mA*mU*mG&mG | | |
| AI-0873 | (18F, PO 17, 10, 3, −3, Mesyl 24, −2, −6) | 117 | 4 |
| (25-1-8) | mG&mC*mC*fC*fC*mA*fG*mCfA*fG*fC*fU*fU*mC*fAmG*fU*fC*f | | |
| | C*fC*fU*mUfU*fC*fU*dC*di#mUfC*mG*mA*mU*mG&mG | | |
| AI-0865 | (17F, 4PO, PN −2 Mesyl 22, 13, 4, −6 MOE 23, 22, 1, −4, −5, −6) | 118 | 4 |
| (23-1-6) | moe(MeC)&moe(MeC)*fC*mA*fG*mCfA*fG*fC*fU&fU*mC*fAmG* | | |
| | fU*fC*fC*fC*fU&mUfU*fC*moeT*dC*di#mUfC*moeG*moeA&moe | | |
| | T | | |
| AI-0866 | (18F, 4PO, PN −2, Mesyl 24, 21, 13, 4, −6, MOE 25, 24, 23, 1, | 119 | 4 |
| (25-1-8) | −4, −6, −7, −8) | | |
| | moeG&moe(MeC)*moe(MeC)*fC&fC*mA*fG*mCfA*fG*fC*fU&fU* | | |
| | mC*fAmG*fU*fC*fC*fC*fU&mUfU*fC*moeT*dC*di#mUfC*moeG* | | |
| | mA*moeT*moeG&moeG | | |
| AI-0867 | (19F, 4PO, PN −2, Mesyl 22, 13, 4, −12, MOE 23, 22, 1, −4, | 120 | 4 |
| (23-1-12) | −10, −11, −12) | | |
| | moe(MeC)&moe(MeC)*fC*mA*fG*mCfA*fG*fC*fU&fU*mC*fAmG* | | |
| | fU*fC*fC*fC*fU&mUfU*fC*moeT*dC*di#mUfC*moeG*mA*fU*mG* | | |
| | fG*mU*moe(MeC)*moeA&moeG | | |
| AI-0875 | (11F, 11PO, MOE 25, 24, 23, 16, 14, 8, 6, 1, −4, −6, −7, −8 | 121 | 4 |
| (25-1-8) | Ome 22, 2, Mesyl 24, 21, 13, 4, −6) | | |
| | moeG&moe(MeC)*moe(MeC)*mC&fC*mA*fGmC*fAmoeG*fCmoe | | |
| | T&fU*mCfA*mG*fU*moe(MeC)*fC*moe(MeC)*fU&mUfUmC*moeT | | |
| | *dC*di#mUfC*moeG*mAmoeT*moeG&moeG | | |
| AI-0876 | (11F, 11PO, MOE 25, 24, 23, 16, 14, 8, 6, 1, −4, −6, −7, −8 | 122 | 4 |
| (25-1-8) | Ome 22, 2, Mesyl 24, −6) | | |
| | moeG&moe(MeC)*moe(MeC)*mC*fC*mA*fGmC*fAmoeG*fCmoe | | |
| | T*fU*mCfA*mG*fU*moe(MeC)*fCmoe(MeC)*fU*mUfUmC*moeT* | | |
| | dC*di#mUfC*moeG*mAmoeT*moeG&moeG | | |
| AI-0877 | (11F, 10PO, MOE 25, 24, 23, 16, 14, 8, 6, 1, −4, −6, −7, −8 | 123 | 4 |
| (25-1-8) | Ome 22, 2, Mesyl 24, −8, −2) | | |
| | moeG&moe(MeC)*moe(MeC)mC*fC*mA*fGmC*fAmoeG*fCmoe | | |
| | T*fU*mCfA*mG*fU*moe(MeC)*fCmoe(MeC)*fU*mUfUmC*moeT* | | |
| | dC*di#mUfC*moeG*mAmoeT*moeG&moeG | | |
| AI-0878 | (11F, 10PO, PN −2, MOE 25, 24, 23, 16, 14, 8, 6, 1, −4, −6, | 124 | 4 |
| (25-1-8) | −7, −8 Ome 22, 2, Mesyl 24, −8) | | |
| | moeG&moe(MeC)*moe(MeC)mC*fC*mA*fGmC*fAmoeG*fCmoe | | |
| | T*fU*mCfA*mG*fU*moe(MeC)*fCmoe(MeC)*fU*mUfUmC*moeT* | | |
| | dC*di#mUfC*moeG*mAmoeT*moeG&moeG | | |
| AI-0879 | (11F, PO 3, −3, PN −2, MOE 25, 24, 23, 16, 14, 8, 6, 1, −4, | 125 | 4 |
| (25-1-8) | −6, −7, −8 Ome 22, 2, Mesyl 24, −8) | | |
| | moeG&moe(MeC)*moe(MeC)*mC*fC*mA*fG*mC*fA*moeG*fC*m | | |
| | oeT*fU*mC*fA*mG*fU*moe(MeC)*fC*moe(MeC)*fU*mUfU*mC*m | | |
| | oeT*dC*di#mUfC*moeG*mA*moeT*moeG&moeG | | |
| AI-0880 | (11F, PN −2, MOE 25, 24, 23, 16, 14, 8, 6, 1, −4, −6, −7, −8 | 126 | 4 |
| (25-1-8) | Ome 22, 2, Mesyl 24, −8) | | |
| | moeG&moe(MeC)*moe(MeC)*mC*fC*mA*fG*mC*fA*moeG*fC*m | | |
| | oeT*fU*mC*fA*mG*fU*moe(MeC)*fC*moe(MeC)*fU*mU*fU*mC* | | |
| | moeT*dC*di#mU*fC*moeG*mA*moeT*moeG&moeG | | |
| AI-0881 | (11F PN −2, MOE 16, 14, 8, 6, 1, −4, Ome 22, 2, Mesyl 24, −8) | 127 | 4 |
| (25-1-8) | mG&mC*mC*mC*fC*mA*fG*mC*fA*moeG*fC*moeT*fU*mC*fA* | | |
| | mG*fU*moe(MeC)*fC*moe(MeC)*fU*mU*fU*mC*moeT*dC*di#m | | |
| | U*fC*moeG*mA*mU*mG&mG | | |
| AI-0882 | (11F, 10PO, PN −2, MOE 16, 14, 8, 6, 1, −4, Ome 22, 2, Mesyl | 128 | 4 |
| (25-1-8) | 24, −8) | | |
| | mG&mC*mCmC*fC*mA*fGmC*fAmoeG*fCmoeT*fU*mCfA*mG*f | | |
| | U*moe(MeC)*fCmoe(MeC)*fU*mUfUmC*moeT*dC*di#mUfC*mo | | |
| | eG*mA*mU*mG&mG | | |
| AI-0883 | (11F, PO 3, −3, PN −2, MOE 16, 14, 8, 6, 1, −4, Ome 22, 2, | 129 | 4 |
| (25-1-8) | Mesyl 24, −8) | | |
| | mG&mC*mC*mC*fC*mA*fG*mC*fA*moeG*fC*moeT*fU*mC*fA* | | |
| | mG*fU*moe(MeC)*fC*moe(MeC)*fU*mUfU*mC*moeT*dC*di#mU | | |
| | fC*moeG*mA*mU*mG&mG | | |
| AI-0884 | (11F, 10PO, MOE 25, 24, 23, 16, 14, 8, 6, 1, −4, −6, −7, −8 | 130 | 4 |
| (25-1-8) | Ome 22, 2, Mesyl 24, −8) | | |
| | moeG&moe(MeC)*moe(MeC)*mC*fC*mA*fG*mCfAmoeG*fC*mo | | |
| | eTfU*mCfA*mG*fUmoe(MeC)*fC*moe(MeC)fU*mUfUmC*moeT* | | |
| | dC*di#mUfC*moeG*mA*moeT*moeG&moeG | | |
| AI-0885 | (11F, 9PO, PN −2, MOE 16, 14, 8, 6, 1, −4, Ome 22, 2, Mesyl | 131 | 4 |
| (25-1-8) | 24, −8) | | |
| | mG&mC*mC*mC*fC*mA*fG*mCfAmoeG*fC*moeTfU*mCfA*mG*f | | |
| | Umoe(MeC)*fC*moe(MeC)fU*mUfUmC*moeT*dC*di#mUfC*mo | | |
| | eG*mA*mU*mG&mG | | |
| AI-0886 | (10F, MOE 18, 17, 8, 3, PO 17, 16, 13, 11, 6, 5, 3, 2, −3, | 132 | 4 |
| (25-1-8) | Mesyl 24, −8) | | |
| | mG&mC*mC*mC*mC*fA*fG*moe(MeC)moeAfG*fC*mUmU#mCf | | |
| | A*fG#fUmoe(MeC)*fC*mCmU#mUmoeTfC*dT*dC*di#mUfC*mG* | | |
| | mA*mU*mG&mG | | |
| AI-0888 | (10F; 10 MOE; PO 17, 16, 13, 11, 8, 5, 3, 2, −3, #12, 4) | 133 | 4 |
| (25-1-8) | moeG*moe(MeC)*moe(MeC)*mC*mC*fA*fG*moe(MeC)moeAfG* | | |
| | fC*mUmU#mCfA*fG#fUmoe(MeC)*fC*mCmU#mUmoeTfC*dT*d | | |
| | C*dimUfC*mG*mA*moeT*moeG&moeG | | |
| AI-0889 | (14F; PO 17, 16, 13, 11, 8, 5, 3, 2, −3, 10 MOE; #−2; Ome 1) | 134 | 4 |
| (25-1-8) | moeG*moe(MeC)*moe(MeC)*fC*fC*mA*fG*mCfAfG*moe(MeC)*f | | |
| | UfU*mCfA*mG*fUmoe(MeC)*fC*moe(MeC)fU*mUfUfC*mU*dC*d | | |
| | i#mUfC*moeG*mA*moeT*moeG&moeG | | |
| AI-0890 | (14F, 10PO, PN −2, MOE 25, 24, 23, 15, 8, 6, −4, −6, −7, −8, | 135 | 4 |
| (25-1-8) | Ome 1) | | |
| | moeG*moe(MeC)*moe(MeC)fC*fC*mA*fGmC*fAfG*moe(MeC)*fU | | |
| | *fU*mCfA*mG*fU*moe(MeC)*fCmoe(MeC)*fU*mUfUfC*mU*dC*d | | |
| | i#mUfC*moeG*mA*moeT*moeG&moeG | | |
| AI-0891 | (14F, 9PO, PN −2, MOE 25, 24, 23, 15, 8, 6, −4, −6, −7, −8, | 136 | 4 |
| (25-1-8) | Ome 1, Mesyl 24, 21, 13, 4, −8) | | |
| | moeG&moe(MeC)*moe(MeC)*fC&fC*mA*fG*mCfAfG*moe(MeC) | | |
| | *fUfU*mCfA*mG*fUmoe(MeC)*fC*moe(MeC)fU&mUfUfC*mU*d | | |
| | C*di#mUfC*moeG*mA*moeT*moeG&moeG | | |
| AI-0892 | (14F, 10PO, PN −2, MOE 25, 24, 23, 15, 8, 6, −4, −6, −7, −8, | 134 | 4 |
| (25-1-8) | Ome 1, Mesyl 24, 21, 13, 4, −8) | | |
| | moeG&moe(MeC)*moe(MeC)fC&fC*mA*fGmC*fAfG*moe(MeC)f | | |
| | UfU*mCfA*mGfU*moe(MeC)*fCmoe(MeC)*fU&mUfUfC*mU*d | | |
| | C*di#mUfC*moeG*mAmoeT*moeG&moeG | | |
| AI-0874 | (11F, 12 2′MOE; Ome 22, 2, & 24, 21, 13, 4, −8; PN −2) | 138 | 4 |
| (25-1-8) | moeG&moe(MeC)*moe(MeC)mC&fC*mA*fGmC*fAmoeG*fCmoe | | |
| | T&fU*mCfA*mG*fU*moe(MeC)*fCmoe(MeC)*fU&mUfUmC*moeT | | |
| | *dC*di#mUfC*moeG*mAmoeT*moeG&moeG | | |
| AI-0894 | (12F, 11 2′MOE, Ome 22, 2; & 24, 21, 13, 4, −8; PN −2) | 139 | 4 |
| (25-1-8) | moeG&moe(MeC)*fCmC&fC*mA*fGmC*fAmoeG*fCmoeT&fU*m | | |
| | CfA*mG*fU*moe(MeC)*fCmoe(MeC)*fU&mUfUmC*moeT*dC*di# | | |
| | mUfC*moeG*mAmoeT*moeG&moeG | | |
| AI-0895 | (13F, 11 2′MOE, Ome 22, 2; & 24, 21, 13, 4, −8; PN −2) | 140 | 4 |
| (25-1-8) | moeG&moe(MeC)*fCmC&fC*mA*fGmC*fAmoeG*fCmoeT&fU*m | | |
| | CfA*mG*fU*moe(MeC)*fCmoe(MeC)*fU&mUfUmC*moeT*dC*di# | | |
| | mUfC*moeG*mAmoeT*moeG&moeG | | |
| AI-0896 | (11F, all MOE instead Ome & 24, 21, 13, 4, −8; PN −2 Ome −2) | 141 | 4 |
| (25-1-8) | moeG&moe(MeC)*moe(MeC)moe(MeC)&fC*moeA*fGmoe(MeC) | | |
| | *fAmoeG*fCmoeT&fU*moe(MeC)fA*moeG*fU*moe(MeC)*fCmoe | | |
| | (MeC)*fU&mUfUmoe(MeC)*moeT*dC*di#mUfC*moeG*moeAmo | | |
| | eT*moeG&moeG | | |
| AI-0897 | (13F, all MOE instead Ome & 24, 21, 13, 4, −8; PN −2 Ome −2) | 142 | 4 |
| (25-1-8) | moeG&moe(MeC)*fCmoe(MeC)&fC*moeA*fGmoe(MeC)*fAmoe | | |
| | G*fCmoeT&fU*moe(MeC)fA*moeG*fU*moe(MeC)*fCmoe(MeC)*f | | |
| | U&mUfUmoe(MeC)*moeT*dC*di#mUfC*moeG*fAmoeT*moeG& | | |
| | moeG | | |
| AI-0824 | mC#mC*mC*fA*fG*moe(MeC)moeAfG*fC*mUmU#mCfA*fG#fUm | 143 | 4 |
| (23-1-6) | oe(MeC)*fC*mCmU#mUmoeTfC*dT*dC*dimUfC*mG*mA#mU | | |

As shown in FIG. 4, oligonucleotides that carried a combination of high levels of 2′-F modifications, PO at at least positions +3 and −3, and mesyl linkages showed improved editing (see, e.g., AI-0852, AI-0857, AI-0860, and AI-0861). For instance, AI-0851 and AI-0852 not only showed an increase in editing relative to AI-0004 but also the shorter ASO AI-0850 and AI-0714. High editing was also observed for AI-0860 (“25-1-8” asymmetry), which comprises 2′F modifications (18F), PO linkages at 3 and-3, and a mesyl linkage modification pattern of “+24, +21, +13, +4, −2, and −8” (AI-0860 carries mesyl linkages at the outermost 5′ and 3′ terminal flanks and 4 internal mesyl linkages). Similarly, AI-0861 (“23-1-12” asymmetry), which also carries high levels of 2′F modifications, PO linkage at positions 3 and −3, and mesyl linkages at 22, 13, 4, −2, and −12, showed editing that was significantly higher when compared to the 45 nt long control, which lacks mesyl linkages.

Furthermore, the inventors were able to show that placement of 2′-MOE modifications within the oligonucleotide and/or within the flanking regions did not compensate for a shortening in oligonucleotide or low levels of mesyl linkages. For Instance, ASOs AI-0875 to AI-0880, which contain a combination of 12 2′-MOE modifications and between 2 to 5 mesyl linkages, showed a significant decrease in target editing. Similarly, constructs AI-0881 to AI-0883, which contain 6 2′-MOE modifications and 2 mesyl linkage exhibited low target editing. Surprisingly, the inventors found that editing efficacy increased for constructs AI-0889, AI-0890, and AI-0891, suggesting that it is desirable to have a balance between the combination of 2′-sugar and internucleoside linkage modifications and their particular placement.

Overall, these results show that the target editing efficacy of shorter oligonucleotides can be improved through the targeted placement of specific 2′-sugar modifications (e.g., 2′F and 2′MOE) and mesyl internucleoside linkage modifications throughout the ASO and its 5′ and 3′ terminal ends.

### Example 5

### Cellular Uptake of GalNAc Modified ASOs in Primary PIZ Mouse Hepatocytes

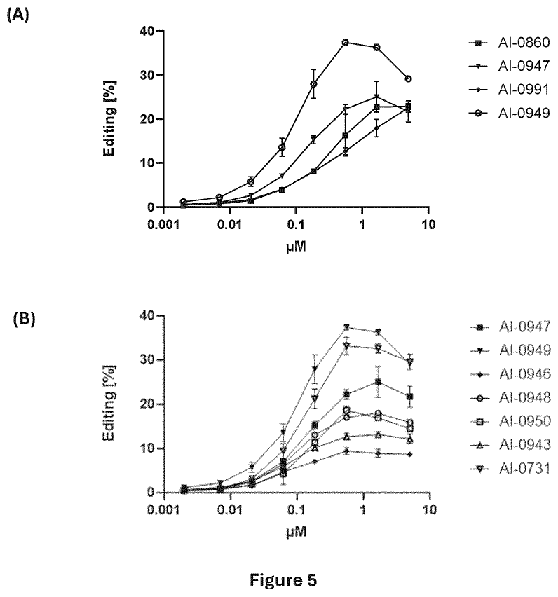

Targeted delivery of oligonucleotides using N-acetylgalactosamine (GalNAc) conjugates that bind to the asialoglycoprotein receptor has become a breakthrough approach in the therapeutic oligonucleotide field. To determine whether 5-terminus modification has an impact on the cellular update of the oligonucleotides of the invention, mesyl modified constructs were 5′-modified to comprise a GalNac residue. The different oligonucleotide constructs and their respective modifications used are listed in Table 5. The results are presented in FIG. 5.

Hepatocyte experiments. Freshly isolated primary mouse hepatocytes from Piz mice, were plated in 86-well collagen-coated plates (Greiner) at a density of 2.5×10 ⁴ cells per well (100 μL per well) in DMEM low glucose (Gibco) supplemented with 10% FBS (Gibco) and 1% penicillin/streptomycin (Gibco) for 4 hours, then they were cultured in William's E Medium (Gibco) supplemented with 1% GlutaMAX™ (Gibco) and 1% penicillin/streptomycin for 24 hours. The hepatocytes were cultured under standard culture conditions at 37° C., 5% CO₂ and in a humidified atmosphere. With the media change, cells were treated with different concentrations of ASOs in 1× PBS (Gibco) for the free uptake, or with a mix of ASO in OptiMEM (Gibco) and 0.3 μL RNAiMAX (Invitrogen) for transfection. All other subsequent steps were the same as with Hela cells.

```
TABLE 5
Oligonucleotide constructs and modifications. * = phosphorothioate (PS); m = 2′-
  OMe; & = (mesyl) methanesulfonyl; f = 2′-fluoro; moe = 2′-O-Methoxyethyl (2′-MOE);
i = inosine; MeC = 5′-methylcytidine; GalNac = N-acetylgalactosamine; # =
  phosphoryl guanidine (PN).
```

| Construct | | Seq. | Fig. |
|---|---|---|---|
| (Asymmetry) | Modification Pattern and Sequence (5′-3′ direction) | ID | No. |
| AI-0860 | mG&mC*mC*fC&fC*mA*fG*mC*fA*fG*fC*fU&fU*mC* | 107 | 5A |
| (25-1-8) | fA*mG*fU*fC*fC*fC*fU&mUfU*fC*fU*dC*di&mUfC*m | | |
| | G*mA*mU*mG&mG | | |
| AI-0947 | (GalNAc) | 144 | 5A |
| (25-1-8) | mG&mC*mC*fC&fC*mA*fG*mC*fA*fG*fC*fU&fU*mC* | | |
| | fA*mG*fU*fC*fC*fC*fU&mUfU*fC*fU*dC*di&mUfC*m | | |
| | G*mA*mU*mG&mG | | |
| AI-0991 | mG&mC*mCfC&fC*mA*fG*mCmoeAfG*fC*moeT&fU* | 145 | 5A |
| (25-1-8) | mCfA*mG*fUmoe(MeC)*fC*moe(MeC)fU&mUmoeTfC | | |
| | *moeT*dC*di&mUfC*moeG*mA*mU*mG&mG | | |
| AI-0949 | (GalNAc) | 146 | 5A |
| (25-1-8) | mG&mC*mCfC&fC*mA*fG*mCmoeAfG*fC*moeT&fU* | | |
| | mCfA*mG*fUmoe(MeC)*fC*moe(MeC)fU&mUmoeTfC | | |
| | *moeT*dC*di&mUfC*moeG*mA*mU*mG&mG | | |
| AI-0946 | (GalNAc) | 147 | 5A |
| (25-1-8) | moeG*moe(MeC)*moe(MeC)*fC*fC*mA*fG*mC*fA*m | | |
| | oeG*fC*moeT*fU*mC*fA*mG*fU*moe(MeC)*fC*moe(MeC) | | |
| | *fU*mUfU*fC*moeT*dC*di#mUfC*moeG*mA*mo | | |
| | eT*moeG*moeG | | |
| AI-0948 | (GalNAc) | 148 | 5A |
| (25-1-8) | mG&mC*mC*fC*fC*mA*fG*mC*fA*fG*fC*fU*fU*mC*f | | |
| | A*mG*fU*fC*fC*fC*fU*mUfU*fC*fU*dC*di&mUfC*mG* | | |
| | mA*mU*mG&mG | | |
| AI-0950 | (GalNAc) | 149 | 5A |
| (23-1-6) | mC&mC*fC*mA*fG*mC*fA*fG*fC*fU&fU*mC*fA*mG*f | | |
| | U*fC*fC*fC*fU&mUfU*fC*fU*dC*di&mUfC*mG*mA&m | | |
| | U | | |
| AI-0943 | (GalNAc) | 150 | 5A |
| (23-1-6) | mC#mC*mC*fA*fG*moe(MeC)moeAfG*fC*mUmU#m | | |
| | CfA*fG#Umoe(MeC)*fC*mCmU#mUmoeTfC*dT*dC* | | |
| | di#mUfC*mG*mA#mU | | |
| AI-0731 | (GalNAc) | 151 | 5A |
| (23-1-6) | mC#mC*mC*fA*fG*mCfA*fG*fC*fU*fU#fC*fAfG#fU*fC | | |
| | *fC*fC*fU*mUfU*fC*fU*dC*di#mUfC*mG*mA#mU | | |

As shown in FIG. 5(A), construct AI-0860 (PO=2,2′-OMe=14, masyl=6, PS=25; 2′-fluoro=18) was efficiently take up by primary Piz mouse hepatocytes. AI-0947 is Identical to AI-0860 aside from the GalNac residue. Addition of the GalNac residue to the 5′ terminus of the oligonucleotide did not impair cellular update. Similarly, construct AI-0991 (PO=9, 2′-MOE=7, MeC=2; 2′-OMe=13, mesyl=6, PS=18, 2′-fluoro=12) was efficiently taken up by primary Piz mouse hepatocytes. Notably, construct AI-0949, which is identical to AI-0991 but carries an additional GalNac residue, showed the highest overall update and editing efficacy of about 40%. These data show that oligonucleotides comprising at least one internucleoside linkage that is a methanesulfonyl (mesyl) linkage shows good editing.

Furthermore, as shown in FIG. 5(B), AI-0949 (PO=9, 2′-MOE=7, MeC=2, 2′-OMe=13, mesyl=6, PS=18, 2′fluoro=12) showed the highest level of editing at the concentrations tested. This editing was higher than that achieved with constructs AI-0946 (PO=2, 2′-MOE=12, MeC=4, 2′-OMe=7, PS=30, mesyl=0, 2′-fluoro=13, PN=1), which contains no mesyl internucleoside linkages Notably, editing was also higher than what was obtained for AI-0848 (PO=2, 2′-OMe=14, mesyl=3, PS=28, 2′-fluoro=18). Shorter oligonucleotide of 30 nt [AI-0950 (PO=2, 2′-OMe=11, mesyl=5, PS=22, 2′-fluoro=17), AI-0943 (PO=9, 2′-MOE=4, MeC=2, 2′OMe=13, mesyl=0, PS=14, 2′-fluoro=10, PN=6), and AI-0731 (PO=4, 2′-OMe=9, mesyl=0, PS=20, 2′fluoro=19, PN=5)] also showed good editing, however, slightly lower than AI-0949.

Overall, these results show that a combination of oligonucleotide modifications that include mesyl internucleoside linkages leads to high levels of editing.

### Example 6

### ASO Transfection at Low Concentration in Primary PiZ Mouse Hepatocytes Allows Efficient Editing

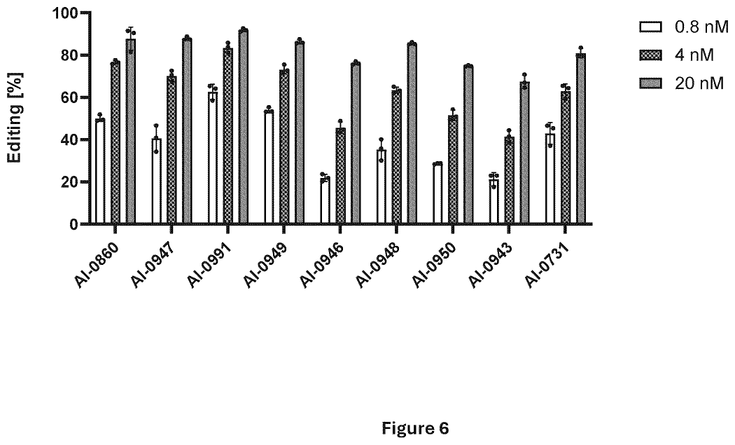

To determine whether efficient editing could be observed at even lower concentrations, different oligonucleotides were tested for their editing efficacy at 0.8 nM, 4 nM and 20 nM. Hepatocyte experiment was conducted as described above. The different oligonucleotide constructs and their respective modifications used are listed in Table 6. The results are presented in FIG. 6.

```
TABLE 6
Oligonucleotide constructs and modifications. * = phosphorothioate (PS); m = 2′-
  OMe; & = (mesyl) methanesulfonyl; f = 2′-fluoro; moe = 2′-O-Methoxyethyl (2′-MOE);
i = inosine; MeC = 5′-methylcytidine; GalNac = N-acetylgalactosamine; # =
  phosphoryl guanidine (PN).
```

| Construct | | Seq. | Fig. |
|---|---|---|---|
| (Asymmetry) | Modification Pattern and Sequence (5′-3′ direction) | ID | No. |
| AI-0860 | mG&mC*mC*fC&fC*mA*fG*mC*fA*fG*fC*fU&fU*mC*fA*mG | 107 | 6 |
| (25-1-8) | *fU*fC*fC*fC*fU&mUfU*fC*fU*dC*di&mUfC*mG*mA*mU*m | | |
| | G&mG | | |
| AI-0947 | (GalNAc) | 144 | 6 |
| (25-1-8) | mG&mC*mC*fC&fC*mA*fG*mC*fA*fG*fC*fU&fU*mC*fA*mG | | |
| | *fU*fC*fC*fC*fU&mUfU*fC*fU*dC*di&mUfC*mG*mA*mU*m | | |
| | G&mG | | |
| AI-0991 | mG&mC*mCfC&fC*mA*fG*mCmoeAfG*fC*moeT&fU*mCfA* | 145 | 6 |
| (25-1-8) | mG*fUmoe(MeC)*fC*moe(MeC)fU&mUmoeTfC*moeT*dC*d | | |
| | i&mUfC*moeG*mA*mU*mG&mG | | |
| AI-0949 | (GalNAc) | 146 | 6 |
| (25-1-8) | mG&mC*mCfC&fC*mA*fG*mCmoeAfG*fC*moeT&fU*mCfA* | | |
| | mG*fUmoe(MeC)*fC*moe(MeC)fU&mUmoeTfC*moeT*dC*d | | |
| | i&mUfC*moeG*mA*mU*mG&mG | | |
| AI-0946 | (GalNAc) | 147 | 6 |
| (25-1-8) | moeG*moe(MeC)*moe(MeC)*fC*fC*mA*fG*mC*fA*moeG*f | | |
| | C*moeT*fU*mC*fA*mG*fU*moe(MeC)*fC*moe(MeC)*fU*mU | | |
| | fU*fC*moeT*dC*di#mUfC*moeG*mA*moeT*moeG*moeG | | |
| AI-0948 | (GalNAc) | 148 | 6 |
| (25-1-8) | mG&mC*mC*fC*fC*mA*fG*mC*fA*fG*fC*fU*fU*mC*fA*mG*f | | |
| | U*fC*fC*fC*fU*mUfU*fC*fU*dCdi&mU*fC*mG*mA*mU*mG& | | |
| | mG | | |
| AI-0950 | (GalNAc) | 149 | 6 |
| (23-1-6) | mC&mC*fC*mA*fG*mC*fA*fG*fC*fU&fU*mC*fA*mG*fU*fC*f | | |
| | C*fC*fU&mUfU*fC*fU*dC*di&mUfC*mG*mA&mU | | |
| AI-0943 | (GalNAc) | 150 | 6 |
| (23-1-6) | mC#mC*mC*fA*fG*moe(MeC)moeAfG*fC*mUmU#mCfA*fG | | |
| | #fUmoe(MeC)*fC*mCmU#mUmoeTfC*dT*dC*di#mUfCmG | | |
| | *mA#mU | | |
| AI-0731 | (GalNAc) | 151 | 6 |
| (23-1-6) | mC#mC*mC*fA*fG*mCfA*fG*fC*fU*fU#fC*fAfG#U*fC*fC*fC | | |
| | *fU*mUfU*fC*fU*dC*di#mUfC*mG*mA#mU | | |

As shown in FIG. 6, transfection of primary PIZ mouse hepatocytes with the different constructs led to high editing. In particular, of the mesyl containing ASOs, oligonucleotide constructs AI-0991 (34 nt) and AI-0849 (34 nt; GalNac), which carry masyl internucleoside linkage modifications at positions +24, +21, +13, +4, −2, and −8 showed high editing even at concentrations of 0.8 nM.

Overall, these results show that shorter oligonucleotides can be modified to comprise specific combinations of mesyl internucleoside linkages at specific positions to improve editing even at lower concentrations. Moreover, the results how that 5′-conjugation of GalNac did not abrogate and/or decrease target editing.

### Example 7

### Candidate Compounds Cause Significant Increase in SERPINA1 Target Editing and M-AAT Protein Levels In Vivo

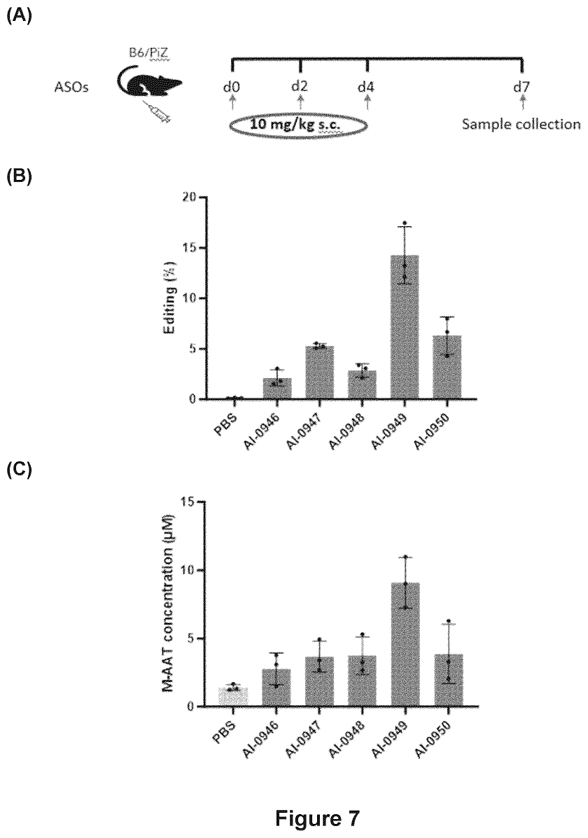

Alpha-1 n deficiency (AATD) is marked by a reduction wild-type (WT) AAT protein, I.e., M-AAT protein, levels. In view of the results obtained in the in vitro studies described in Example 6 above, it was decided to further investigate the effect of lead candidate ASOs on SERPINA1 target editing (%) and M-AAT protein expression levels in vivo. Specifically, AI-0946. AI-0947, AI-0948, and AI-0849 (all having a “25-1-8” asymmetry) and AI-0950 (“23-1-6” asymmetry) were examined for the efficacy in B6/PiZ mice (FIG. 7).

In vivo studies: Studies were performed in mice by Synovo GmbH (Tübingen, Germany) in accordance with procedures approved by the Regional Council (Regierungspräsidium Tübingen, BW, Germany). Mice were housed on a 12:12 light-dark cycle, with ad libitum access to food and water. Mice expressing the human SERPINA1^{8342X} transgene in C57BL/6J background were described previously and were provided by Prof. Jeffrey Teckman's laboratory (Saint Louis University). Mice homozygous for the human transgene (PIZZ) were crossbred with wild-type C57BL/6) and offspring was used in all experiments. Eight-to-ten-week-old male and female mice were subcutaneously injected with either 10 mg/kg or 5 mg/kg depending on the experiment. ASO dissolved in PBS or PBS only as indicated. Injections were performed on experimental day 0, day 2 and day 4. Animals were sacrificed 7 days after the first dose and livers were collected and snap-frozen. Tissues were lysed in buffer RLT (RNeasy mini kit, Qiagen) with a bead homogenizer (Bead Mill Max, VWR) and 1.4 mm ceramic beads, at 4.5 m/s for 30 sec. The lysates were used for total RNA purification using RNeasy mini kit (Qiagen) with an on-column DNase I digest. The obtained RNA was then processed as described above to perform NGS amplicon sequencing and determining the RNA editing yield.

Here, as shown in FIG. 7(A), blood samples were initially taken from untreated animals on day (d) 0. Mice were subsequently administered the individual ASO candidate at a concentration of 3×10 mg/kg at day 0, day 2, and day 4 via subcutaneous (s.c.) injection. Tissue samples were collected on day 7 and subsequently analysed for target editing % and M-AAT protein levels. The results are shown in FIG. 7(B) and (C). PBS-treated animals served as negative control. The different oligonucleotide constructs, their respective modification pattern and sequence are listed in Table 7.

TABLE 7
Oligonucleotide constructs and modifications. d = 2′-H (deoxyribose; DNA);
* = phosphorothioate (PS); m = 2′-OMe; & = (mesyl) methanesulfonyl; f = 2′-fluoro;
moe = 2′-O-Methoxyethyl (2′-MOE); i = inosine; MeC = 5′-methylcytidine; # =
phosphoryl guanidine (PN) ; SbU = iso-Uridine; GalNac = N-acetylgalactosamine.

| Construct | | Seq. | Fig. |
|---|---|---|---|
| (Asymmetry) | Modification Pattern and Sequence (5′-3′ direction) | ID | No. |
| AI-0946 | (GalNAc)moeG*moe(MeC)*moe(MeC)*fC*fC*mA*fG*mC*fA* | 147 | 7 |
| (25-1-8) | moeG*fC*moeT*fU*mC*fA*mG*fU*moe(MeC)*fC*moe(MeC)*f | | |
| | U*mUfU*fC*moeT*dC*di#mUfC*moeG*mA*moeT*moeG*moe | | |
| | G | | |
| AI-0947 | (GalNAc)mG&mC*mC*fC&fC*mA*fG*mC*fA*fG*fC*fU&fU*mC | 144 | 7 |
| (25-1-8) | *fA*mG*fU*fC*fC*fC*fU&mUfU*fC*fU*dC*di#mUfC*mG*mA* | | |
| | mU*mG&mG | | |
| AI-0948 | (GalNAc)mG&mC*mC*fC&fC*mA*fG*mC*fA*fG*fC*fU*fU*m | 148 | 7 |
| (25-1-8) | C*fA*mG*fU*fC*fC*fC*fU*mUfU*fC*fU*dC*di&mUfC*mG*mA | | |
| | *mU*mG&mG | | |
| AI-0949 | (GalNAc)mGmC*mCfC&fC*mA*fG*mCmoeAfG*fC*moeT& | 146 | 7 |
| (25-1-8) | fU*mCfA*mG*fUmoe(MeC)*fCmoe(MeC)fU&mUmoeTfC*m | | |
| | oeT*dC*di&mUfC*moeG*mA*mU*mG&mG | | |
| AI-0950 | (GalNAc)mC&mC*fC*mA*fG*mC*fA*fG*fC*fU&fU*mC*fA*m | 149 | 7 |
| (23-1-6) | G*fU*fC*fC*fC*fU&mUfU*fC*fU*dC*di&mUfC*mG*mA&mU | | |

As shown in FIG. 7, highest in vivo editing and M-AAT levels were observed with AI-0949 (25-1-8). This construct contains a combination mesyl and PO linkages and 2′-F and 2′-MOE modifications. Specifically, the mesyl linkage modification pattern for this ASD candidate is “+24, +21, +13, +4, −2, −8”.

Overall, these results show the benefits of mesyl modification is also exhibited on in vivo administration of the ASOs of the invention.

### Example 8

### PO and PS Internucleoside Linkage Replacement by Mesyl Internucleoside Linkages

Different SERPINA1-specific ASOs of the same length and asymmetry were generated in which the PO or PS linkage was substituted with a mesyl linkage. ASO AI-1987 (25-1-8) was used as a starting point comprising 3 mesyl linkage modifications. One mesyl linkage is located at the outermost 5′ flanking position, one mesyl linkage is located at the outermost 3′ flanking position. and one mesyl linkage is located within the ASO, i.e., mesyl linkages are located at positions +24, −2, and −8 of the ASO. A “mesyl walk” was conducted by replacing either a PO or PS linkage within the ASO so as to introduce an additional fourth mesyl linkage into the ASO. The different oligonucleotide constructs, their respective modification pattern and sequence are listed in Table 8. ASOs were tested at a concentration of 20 M and 4 nM.

TABLE 8
Oligonucleotide and their modifications used for mesyl walk. d = 2′-H
(deoxyribose; DNA); * = phosphorothioate (PS); m = 2′-OMe; & = (mesyl) methanesulfonyl; f =
2′-fluoro; moe = 2′-O-Methoxyethyl (2′-MOE); i = inosine; MeC =
5′-methylcytidine; # = phosphoryl guanidine (PN).

| Construct | | Seq. | Fig. |
|---|---|---|---|
| (Asymmetry) | Modification Pattern and Sequence (5′ to 3′ direction) | ID | No. |
| AI-1987 | mG&mC*mCfC*fC*mA*fG*mCmoeAfG*fC*moeT*fUmCfAmG*f | 152 | 8 |
| | Umoe(MeC)*fCmoe(MeC)fU*mUmoeTfC*moeT*dC*di&mUf* | | |
| | moeG*mA*mU*mG&mG | | |
| AI-2085 | mG&mC&mCfC*fC*mA*fG*mCmoeAfG*fC*moeT*fUmCfAmG*f | 153 | 8 |
| (& at +23) | Umoe(MeC)*fCmoe(MeC)fU*mUmoeTfC*moeT*dC*di&mUfC* | | |
| | moeG*mA*mU*mG&mG | | |
| AI-2530 | mG&mC*mC&fC*fC*mA*fG*mCmoeAfG*fC*moeT*fUmCfAmG* | 154 | 8 |
| (& at +22) | fUmoe(MeC)*fCmoe(MeC)fU*mUmoeTfC*moeT*dC*di&mUfC* | | |
| | moeG*mA*mU*mG&mG | | |
| AI-2485 | mG&mC*mCfC&fC*mA*fG*mCmoeAfG*fC*moeT*fUmCfAmG | 155 | 8 |
| (& at +21) | *fUmoe(MeC)*fCmoe(MeC)fU*mUmoeTfC*moeT*dC*di&mUf | | |
| | C*moeG*mA*mU*mG&mG | | |
| AI-2486 | mG&mC*mCfC*fC&mA*fG*mCmoeAfG*fC*moeT*fUmCfAmG | 156 | 8 |
| (& at +20) | *fUmoe(MeC)*fCmoe(MeC)fU*mUmoeTfC*moeT*dC*di&mUf | | |
| | C*moeG*mA*mU*mG&mG | | |
| AI-2487 | mG&mC*mCfC*fC*mA&fG*mCmoeAfG*fC*moeT*fUmCfAmG | 157 | 8 |
| (& at +19) | *fUmoe(MeC)*fCmoe(MeC)fU*mUmoeTfC*moeT*dC*di&mUf | | |
| | C*moeG*mA*mU*mG&mG | | |
| AI-2488 | mG&mC*mCfC*fC*mA*fG&mCmoeAfG*fC*moeT*fUmCfAmG | 158 | 8 |
| (& at +18) | *fUmoe(MeC)*fCmoe(MeC)fU*mUmoeTfC*moeT*dC*di&mUf | | |
| | C*moeG*mA*mU*mG&mG | | |
| AI-2531 | mG&mC*mCfC*fC*mA*fG*mC&moeAfG*fC*moeT*fUmCfAmG* | 159 | 8 |
| (& at +17) | fUmoe(MeC)*fCmoe(MeC)fU*mUmoeTfC*moeT*dC*di&mUfC* | | |
| | moeG*mA*mU*mG&mG | | |
| AI-2652 | mG&mC*mCfC*fC*mA*fG*mCmoeA&fG*fC*moeT*fUmCfAmG* | 160 | 8 |
| (& at +16) | fUmoe(MeC)*fCmoe(MeC)fU*mUmoeTfC*moeT*dC*di&mUfC* | | |
| | moeG*mA*mU*mG&mG | | |
| AI-2489 | mG&mC*mCfC*fC*mA*fG*mCmoeAfG&fC*moeT*fUmCfAmG | 161 | 8 |
| (& at +15) | *fUmoe(MeC)*fCmoe(MeC)fU*mUmoeTfC*moeT*dC*di&mUf | | |
| | C*moeG*mA*mU*mG&mG | | |
| AI-2490 | mG&mC*mCfC*fC*mA*fG*mCmoeAfG*fC&moeT*fUmCfAmG | 162 | 8 |
| (& at +14) | *fUmoe(MeC)*fCmoe(MeC)fU*mUmoeTfC*moeT*dC*di&mUf | | |
| | C*moeG*mA*mU*mG&mG | | |
| AI-2491 | mG&mC*mCfC*fC*mA*fG*mCmoeAfG*fC*moeT&fUmCfAmG | 163 | 8 |
| (& at +13) | *fUmoe(MeC)*fCmoe(MeC)fU*mUmoeTfC*moeT*dC*di&mUf | | |
| | C*moeG*mA*mU*mG&mG | | |
| AI-2653 | mG&mC*mCfC*fC*mA*fG*mCmoeAfG*fC*moeT*fU&mCfAmG* | 164 | 8 |
| (& at +12) | fUmoe(MeC)*fCmoe(MeC)fU*mUmoeTfC*moeT*dC*di&mUfC* | | |
| | moeG*mA*mU*mG&mG | | |
| AI-2654 | mG&mC*mCfC*fC*mA*fG*mCmoeAfG*fC*moeT*fUmC&fAmG* | 165 | 8 |
| (& at +11) | fUmoe(MeC)*fCmoe(MeC)fU*mUmoeTfC*moeT*dC*di&mUfC* | | |
| | moeG*mA*mU*mG&mG | | |
| AI-2655 | mG&mC*mCfC*fC*mA*fG*mCmoeAfG*fC*moeT*fUmCfAmG* | 166 | 8 |
| (& at +10) | fUmoe(MeC)*fCmoe(MeC)fU*mUmoeTfC*moeT*dC*di&mUfC* | | |
| | moeG*mA*mU*mG&mG | | |
| AI-2492 | mG&mC*mCfC*fC*mA*fG*mCmoeAfG*fC*moeT*fUmCfAmG&f | 167 | 8 |
| (& at +9) | Umoe(MeC)*fCmoe(MeC)fU*mUmoeTfC*moeT*dC*di&mUfC* | | |
| | moeG*mA*mU*mG&mG | | |
| AI-2656 | mG&mC*mCfC*fC*mA*fG*mCmoeAfG*fC*moeT*fUmCfAmG*f | 168 | 8 |
| (& at +8) | U&moe(MeC)*fCmoe(MeC)fU*mUmoeTfC*moeT*dC*di&mUfC | | |
| | *moeG*mA*mU*mG&mG | | |
| AI-2493 | mG&mC*mCfC*fC*mA*fG*mCmoeAfG*fC*moeT*fUmCfAmG*f | 169 | 8 |
| (& at +7) | Umoe(MeC)&fCmoe(MeC)fU*mUmoeTfC*moeT*dC*di&mUfC* | | |
| | moeG*mA*mU*mG&mG | | |
| AI-2657 | mG&mC*mCfC*fC*mA*fG*mCmoeAfG*fC*moeT*fUmCfAmG*f | 170 | 8 |
| (& at +6) | Umoe(MeC)*fC&moe(MeC)fU*mUmoeTfC*moeT*dC*di&mUfC | | |
| | *moeG*mA*mU*mG&mG | | |
| AI-2658 | mG&mC*mCfC*fC*mA*fG*mCmoeAfG*fC*moeT*fUmCfAmG*f | 171 | 8 |
| (& at +5) | Umoe(MeC)*fCmoe(MeC)&fU*mUmoeTfC*moeT*dC*di&mUfC | | |
| | *moeG*mA*mU*mG&mG | | |
| AI-2494 | mG&mC*mCfC*fC*mA*fG*mCmoeAfG*fC*moeT*fUmCfAmG*f | 172 | 8 |
| (& at +4) | Umoe(MeC)*fCmoe(MeC)fU&mUmoeTfC*moeT*dC*di&mUfC* | | |
| | moeG*mA*mU*mG&mG | | |
| AI-2659 | mG&mC*mCfC*fC*mA*fG*mCmoeAfG*fC*moeT*fUmCfAmG*f | 173 | 8 |
| (& at +3) | Umoe(MeC)*fCmoe(MeC)fU*mU&moeTfC*moeT*dC*di&mUfC | | |
| | *moeG*mA*mU*mG&mG | | |
| AI-2660 | mG&mC*mCfC*fC*mA*fG*mCmoeAfG*fC*moeT*fUmCfAmG*f | 174 | 8 |
| (& at +2) | Umoe(MeC)*fCmoe(MeC)fU*mUmoeT&fC*moeT*dC*di&mUfC | | |
| | *moeG*mA*mU*mG&mG | | |
| AI-2495 | mG&mC*mCfC*fC*mA*fG*mCmoeAfG*fC*moeT*fUmCfAmG*f | 175 | 8 |
| (& at +1) | Umoe(MeC)*fCmoe(MeC)fU*mUmoeTfC&moeT*dC*di&mUf | | |
| | C*moeG*mA*mU*mG&mG | | |
| AI-2496 | mG&mC*mCfC*fC*mA*fG*mCmoeAfG*fC*moeT*fUmCfAmG*f | 176 | 8 |
| (& at +0) | Umoe(MeC)*fCmoe(MeC)fU*mUmoeTfC*moeT&dC*di&mUf | | |
| | C*moeG*mA*mU*mG&mG | | |
| AI-2497 | mG&mC*mCfC*fC*mA*fG*mCmoeAfG*fC*moeT*fUmCfAmG*f | 177 | 8 |
| (& at −1) | Umoe(MeC)*fCmoe(MeC)fU*mUmoeTfC*moeT*dC&di&mUf | | |
| | C*moeG*mA*mU*mG&mG | | |
| AI-2861 | mG&mC*mCfC*fC*mA*fG*mCmoeAfG*fC*moeT*fUmCfAmG*f | 178 | 8 |
| (& at −3) | Umoe(MeC)*fCmoe(MeC)fU*mUmoeTfC*moeT*dC*di&mU&fC | | |
| | *moeG*mA*mU*mG&mG | | |
| AI-2496 | mG&mC*mCfC*fC*mA*fG*mCmoeAfG*fC*moeT*fUmCfAmG* | 179 | 8 |
| (& at −4) | fUmoe(MeC)*fCmoe(MeC)fU*mUmoeTfC*moeT*dC*di&mU&fC | | |
| | &moeG*mA*mU*mG&mG | | |
| AI-2499 | mG&mC*mCfC*fC*mA*fG*mCmoeAfG*fC*moeT*fUmCfAmG* | 180 | 8 |
| (& at −5) | fUmoe(MeC)*fCmoe(MeC)fU*mUmoeTfC*moeT*dC*di&mUfC | | |
| | *moeG&mA*mU*mG&mG | | |
| AI-2500 | mG&mC*mCfC*fC*mA*fG*mCmoeAfG*fC*moeT*fUmCfAmG* | 181 | 8 |
| (& at −6) | fUmoe(MeC)*fCmoe(MeC)fU*mUmoeTfC*moeT*dC*di&mUfC | | |
| | *moeG*mA&mU*mG&mG | | |
| AI-2084 | mG&mC*mCfC*fC*mA*fG*mCmoeAfG*fC*moeT*fUmCfAmG*f | 182 | 8 |
| (& at −7) | Umoe(MeC)*fCmoe(MeC)fU*mUmoeTfC*moeT*dC*di&mUfC* | | |
| | moeG*mA*mU&mG&mG | | |

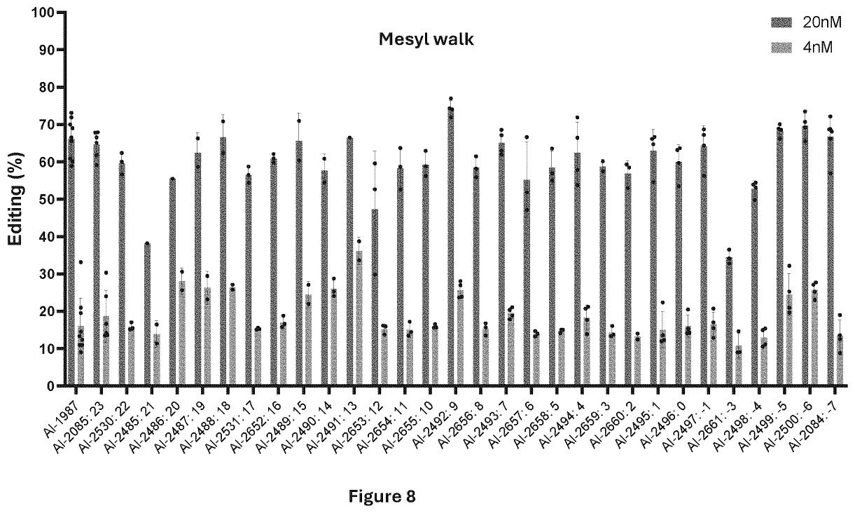

As shown in FIG. 8, generally replacing a single PO or PS linkage with a mesyl linkage only marginally changed the editing efficacy of the different ASOs and had little impact on the overall editing efficacy. Notably, there was, e.g., a more pronounced decrease in editing for both concentrations when the mesyl linkage was placed at position −3 (see, AI-2681). Interestingly, for ASOs; AI-2491 (additional internal mesyl linkage at +13) and AI-2492 (additional internal mesyl linkage at +9), there was good editing at 20 nM and 4 nM.

Overall, these results show that replacing a single PO or PS linkage with a mesyl linkage in an ASO that already contains 3 mesyl linkages (positions +24, −2, and −8) only slightly impacted on the editing efficacy of the different ASOs. In other words, a further mesyl linkage could generally be introduced to replace a PO or PS linkage without negatively impacting editing. This further internal mesyl linkage seemed to provide a putative stability increase.

### Example 9

### Mesyl Linkage Modification Provides Improved Target Editing Compared to Methylphosphonate (MP) Linkage Modification In Vitro and In Vivo

Methylphosphonate (MP) oligonucleotides (MPOs) were among the first oligonucleotide analogues reported to inhibit protein synthesis; however, unlike natural phosphodiester (PO) oligonucleotides, MPOs contain chiral linkages (Reynolds et al., 1996). To determine whether there is a difference in editing efficacy between ASOs containing one or more MP linkage or mesyl linkage modifications. different ASO constructs were generated containing either two or three mesyl or MP linkages. Specifically, each of the constructs carried either mesyl or MP linkages in the 5′ and 3′ flanking regions (at positions +24 and −8). Constructs AI-2931. AI-2938, AI-2937, and AI-2940 were generated each carrying an additional internal mesyl or MP linkage modification at position ˜2.

In vivo studies were performed as described under Example 7 above. Animals were treated with the respective test construct at a concentration of 3×5 mg/kg.

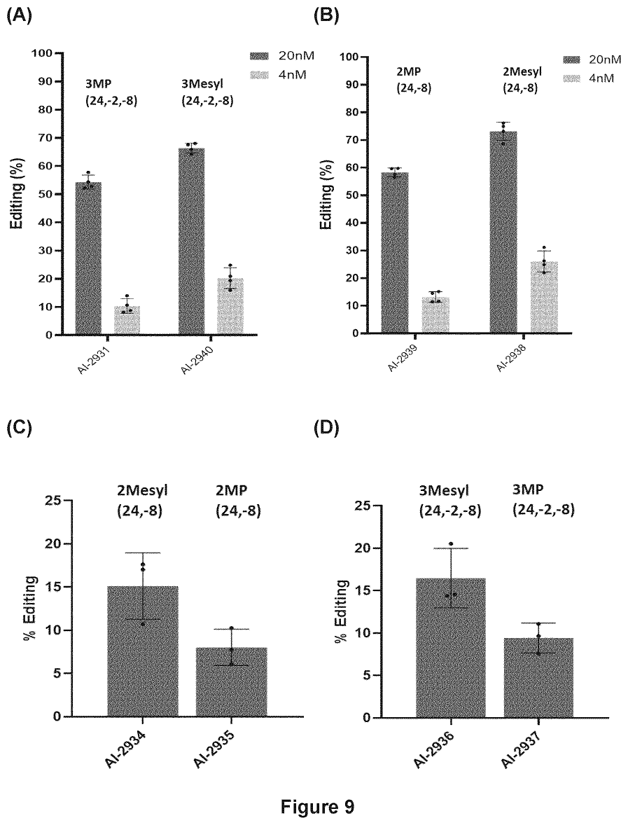

The in vitro results are shown in FIGS. 9(A) and (B). The in vivo results are shown in FIGS. 9(C) and (D). ASOs were tested at a concentration of 20 nM and 4 nM. The different oligonucleotide constructs, their respective modification pattern and sequence are listed in Table 9.

TABLE 9
Oligonucleotide constructs and modifications. d = 2′-H(deoxyribose; DNA);
* = phosphorothioate (PS); m = 2′-OMe; & = (mesyl) methanesulfonyl; f = 2′-fluoro;
moe = 2′-O-Methoxyethyl (2′-MOE); i = inosine; MeC = 5′-methylcytidine; ? or MP =
Methylphosphonate.

| Construct | | Seq. | Fig. |
|---|---|---|---|
| (Asymmetry) | Modification Pattern and Sequence (5′-3′ direction) | ID | No. |
| AI-2931 | mG?mC*mCfC*fC*mA*fG*mCmoeAfG*fC*moeT*fU*mCfA*m | 183 | 9 |
| (25-1-8) | G*fUmoe(MeC)*fC*moe(MeC)fU*mUmoeTfC*moeT*dC*(mi?) | | |
| | mUfC*moeG*mA*mU*mG?mG | | |
| AI-2934 | mG&mC*mCfC*fC*mA*fG*mCmoeAfG*fC*moeT*fU*mCfA*m | 184 | 9 |
| (25-1-8) | G*fUmoe(MeC)*fC*moe(MeC)fU*mUmoeTfC*moeT*dC*m | | |
| | UfC*moeG*mA*mU*mG&mG(GalNac) | | |
| AI-2935 | mG?mC*mCfC*fC*mA*fG*mCmoeAfG*fC*moeT*fU*mCfA*m | 185 | 9 |
| (25-1-8) | G*fUmoe(MeC)*fC*moe(MeC)fU*mUmoeTfC*moeT*dC*m | | |
| | UfC*moeG*mA*mU*mG?mG(GalNac) | | |
| AI-2936 | mG&mC*mCfC*fC*mA*fG*mCmoeAfG*fC*moeT*fU*mCfA*m | 186 | 9 |
| (25-1-8) | G*fUmoe(MeC)*fC*moe(MeC)fU*mUmoeTfC*moeT*dC*mi& | | |
| | mUfC*moeG*mA*mU*mG&mG(GalNac) | | |
| AI-2937 | mG?mC*mCfC*fC*mA*fG*mCmoeAfG*fC*moeT*fU*mCfA*m | 187 | 9 |
| (25-1-8) | G*fUmoe(MeC)*fC*moe(MeC)fU*mUmoeTfC*moeT*dC*mi?m | | |
| | UfC*moeG*mA*mU*mG?mG(GalNac) | | |
| AI-2938 | mG&mC*mCfC*fC*mA*fG*mCmoeAfG*fC*moeT*fU*mCfA*m | 188 | 9 |
| (25-1-8) | G*fUmoe(MeC)*fC*moe(MeC)fU*mUmoeTfC*moeT*dC*di*m | | |
| | UfC*moeG*mA*mU*mG&mG | | |
| AI-2939 | mG?mC*mCfC*fC*mA*fG*mCmoeAfG*fC*moeT*fU*mCfA*m | 189 | 9 |
| (25-1-8) | G*fUmoe(MeC)*fC*moe(MeC)fU*mUmoeTfC*moeT*dC*di*m | | |
| | UfC*moeG*mA*mU*mG?mG | | |
| AI-2940 | mG&mC*mCfC*fC*mA*fG*mCmoeAfG*fC*moeT*fU*mCfA*m | 190 | 9 |
| (25-1-8) | G*fUmoe(MeC)*fC*moe(MeC)fU*mUmoeTfC*moeT*dC*mi& | | |
| | mUfC*moeG*mA*mU*mG&mG | | |

As shown in FIG. 9(A), ASO candidate AI-2940, which carries mesyl linkages at positions +24, −2 and +8, showed significantly higher editing (%) than the methylphosphonate-containing AI-2931 ASO in vitro. This significant increase was observed for both concentrations tested. Similarly, placing mesyl linkages at only the terminal 5′ and 3′ flanking positions conferred improved editing when compared to methylphosphonate linkages at these positions (AI-2938 vs AI-2939) (FIG. 9(B). Again, this was observed for both concentrations tested.

Furthermore, the editing effect was assessed in vivo. As shown in FIGS. 9(C) and (D), a similar trend was observed in vivo. ASO constructs carrying mesyl linkage modifications showed improved editing when compared to constructs. carrying MP linkages in the same positions.

Overall, these results show that the placement of mesyl linkage modifications at the 5′ and 3′ flanking regions significantly increased target editing when compared to ASOs with methylphosphonate linkages at the same positions. Further, it was shown that, in this context, having an internal mesyl linkage at position −2 does not significantly impact on the improved editing efficacy of ASOs that contain mesyl linkages in the terminal flanking regions. The results provide a clear comparison between the two types of linkages and show an improved performance of mesyl linkages over methylphosphonate linkages in enhancing target editing not only in vitro but also in vivo.

### Example 10

### Combination of Mesyl, PO and PS Linkage Modifications Enhances Target Editing In Vivo

To verify the improved effect of mesyl linkage modifications on the ability of the ASO to mediate in vivo A-to-I editing and optimise target editing, numerous ASOs were generated differing in the placement of mesyl, PO and PS linkage modifications. The editing efficacy of the different constructs was assessed In vivo.

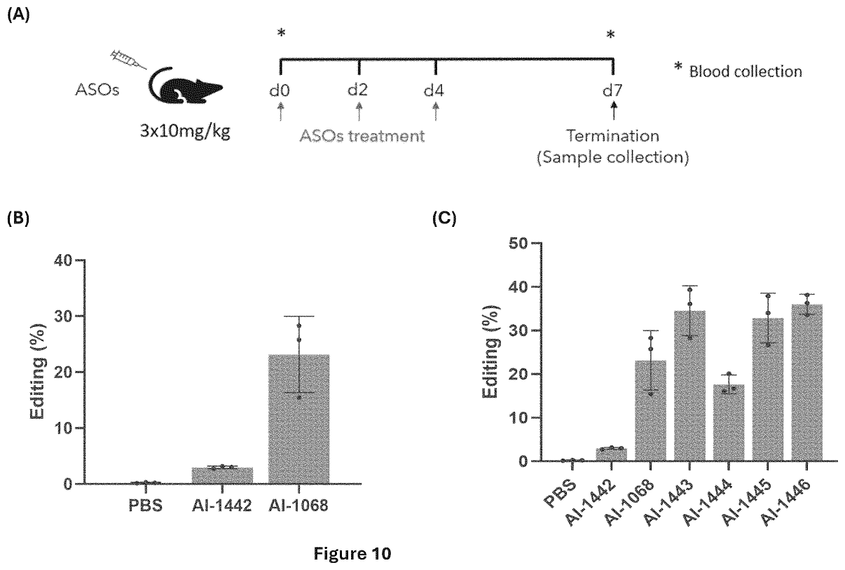

In vivo Study: In vivo studies were performed as described under Example 7 above and ASOs used at a concentration 3×10 mg/kg. Briefly, blood samples were taken from untreated mice on day (d) 0 (FIG. 10(A), * signifies blood sample collection). Mice were subsequently given the respective ASO at a concentration of 3×10 mg/kg at day 0, day. 2 and day 4 via subcutaneous (s.c.) administration. A second blood sample was taken at day 7. Samples were subsequently analysed for editing %. The results are shown in FIG. 10(B) and (C). PBS-treated animals and AI-1442-treated animals served as negative control. The different oligonucleotide constructs, their respective modification pattern and sequence are listed in Table 10.

TABLE 10
Oligonucleotide constructs and modifications. d = 2′-H(deoxyribose; DNA);
* = phosphorothioate (PS); m = 2′-OMe; & = (mesyl) methanesulfonyl; f = 2′-fluoro;
moe = 2′-O-Methoxyethyl (2′-MOE); i = inosine; MeC = 5′-methylcytidine; # = phosphoryl
guanidine (PN); SbU = iso-Uridine; GalNac = N-acetylgalactosamine.

| Construct | | Seq. | Fig. |
|---|---|---|---|
| (Asymmetry) | Modification Pattern and Sequence (5′-3′ direction) | ID | No. |
| AI-1442 | mG*mC*mCfC*fC*mA*fG*mCmoeAfG*fC*moeT*fU*mCfA*m | 191 | 10 |
| (25-1-8) | G*fUmoe(MeC)*fC*moe(MeC)fU*mUmoeTfC*moeT*dC*dimU | | |
| | fC*moeG*mA*mU*mG*mG(GalNac) | | |
| AI-1068 | mG&mC*mCfC&fC*mA*fG*mCmoeAfG*fC*moeT&fU*mCfA* | 192 | 10 |
| (25-1-8) | mG*fUmoe(MeC)*fC*moe(MeC)fU&mUmoeTfC*moeT*dC*di | | |
| | &mUfC*moeG*mA*mU*mG&mG(GalNac) | | |
| AI-1443 | mG&mC*mCfC&fC*mA*fG*mCmoeAfG*fC*moeT&fU*mCfA* | 193 | 10 |
| (25-1-8) | mG*fUmoe(MeC)*fC*moe(MeC)fU&mUmoeTfC*T*d(Sbu)*d | | |
| | i&mUfC*moeG*mA*mU*mG&mG(GalNac) | | |
| AI-1444 | mG&mC*mCfC&fC*mA*fGmCmoeAfG*fC*moeT&fUmCfA*m | 194 | 10 |
| (25-1-8) | G*fUmoe(MeC)*fCmoe(MeC)fU&mUmoeTfC*moeT*dC*di& | | |
| | mUfC*moeG*mAmU*mG&mG(GalNac) | | |
| AI-1445 | mG&mC*mCfC&fC*mA*fG*mCmoeAfG*fC*moeT&fUmCfAm | 195 | 10 |
| (25-1-8) | G*fUmoe(MeC)*fCmoe(MeC)fU&mUmoeTfC*moeT*dC*di& | | |
| | mUfC*moeG*mA*mU*mG&mG(GalNac) | | |
| AI-1446 | mG&mCmCfC&fC*mA*fG*mCmoeAfG*fC*moeT&fUmCfAm | 196 | 10 |
| (25-1-8) | G*fUmoe(MeC)*fCmoe(MeC)fU&mUmoeTfC*moeT*dC*di& | | |
| | mUfC*moeG*mA*mU*mG&mG(GalNac) | | |

As shown in FIG. 10(B), administration of AI-1068 led to a significant increase in target editing compared to PBS-or AI-1442-treated control animals. Moreover, it was shown that editing efficacy could further be increased by optimising the ratio of nucleotide modifications and internucleoside linkage modifications comprising mesyl, PO and PS linkages. That is, AI-1443, which differs from AI-1088 in that it carries a SbU modification at position zero (N₀) and no MOE modification at position +1, caused an in increased target editing (FIG. 10(C), ASO candidates AI-1445 and AI-1446, all of which share the same mesyl modification pattern as AI-1068 (mesyl modifications at the terminal 5′ and 3′ flanking positions +24 and−8 respectively, and internal positions +21. +13. +4 and-2) but comprise higher levels of PO and lower levels of PS linkages showed improved editing (FIG. 10(C)). There was no significant difference in editing between AI-1445 and AI-1446. However, a significant decrease in editing was observed for AI-1444 when compared to AI-1446 and AI-1446. Notably, while ASO candidates AI-1444 and AI-1446 share the same number and positions of 2′MOE modifications, the same number and positions of mesyl linkages, and numbers of PO and PS linkages, they differ in the positioning of such PO and PS linkages.

Overall, these results indicate that the editing efficacy of ASOs comprising identical mesyl modifications may further be increased by optimising the PS and PO backbone modification pattern.

### Example 11

### Mesyl Linkages at the 5′ and 3′ Terminal Flanking Regions Improves Editing Compared to PN or 2′OME Modifications

To investigate the impact of additional 2′-sugar modifications at the 5′ and 3′ terminal positions or internucleoside linkage modifications at the 5′ and 3′ flanking regions, ASO candidates were generated harbouring either 2′-MOE modifications or PN internucleoside linkages. Three differently modified ASOs were generated and the editing efficacy of the different ASOs was assessed in vivo.

In vivo study. In vivo studies were performed as described under Example 7 above and ASOs used at a concentration 3×10 mg/kg.

AI-1068 comprises a mesyl linkage modification pattern of “+24, +21, +13, +4, −2, −8” (Table 11). AI-1207 is identical to AI-1068; however, it does not carry any mesyl linkage modifications and contains 2′MOE modifications at the terminal three nucleotides of the 5′ and 3′ termini. AI-1208 is identical to AI-1068 but only carries a total of 3 mesyl linkage modifications at the outermost 5′ and 3′ flanking positions, i.e., at positions +24 and-8, and internally at position-2, but not at positions +21, +13, and +4. AI-1209 is identical to AI-1068 but carries PN linkages at positions +24, +21, +13, +4,-2,-8 instead of mesyl linkages. PBS was used as negative control. The different oligonucleotide constructs, their respective modification patter and sequence are listed in Table 11.

TABLE 11
Oligonucleotide constructs and modifications. d = 2′-H(deoxyribose; DNA);
* = phosphorothioate (PS); m = 2′-OMe; & = (mesyl) methanesulfonyl; f = 2′-fluoro;
moe = 2′-O-Methoxyethyl (2′-MOE); i = inosine; MeC = 5′-methylcytidine; # = phosphoryl
guanidine (PN); GalNac = N-acetylgalactosamine.

| Construct | | Seq. | Fig. |
|---|---|---|---|
| (Asymmetry) | Modification Pattern and Sequence (5′-3′ direction) | ID | No. |
| AI-1207 | moeG*moe(MeC)*moe(MeC)fC*fC*mA*fG*mCmoeAfG*fC*m | 197 | 11 |
| (25-1-8) | oeT*fU*mCfA*mG*fUmoe(MeC)*fC*moe(MeC)fU*mUmoeTfC | | |
| | *moeT*dC*dimUfC*moeG*mA*moeT*moeG*moeG(GalNac) | | |
| AI-1208 | mG&mC*mCfC*fC*mA*fG*mCmoeAfG*fC*moeT*fU*mCfA* | 198 | 11 |
| (25-1-8) | mG*fUmoe(MeC)*fC*moe(MeC)fU*mUmoeTfC*moeT*dC*di | | |
| | &mUfC*moeG*mA*mU*mG&mG(GalNac) | | |
| AI-1068 | mG&mC*mCfC&fC*mA*fG*mCmoeAfG*fC*moeT&fU*mCfA* | 192 | 11 |
| (25-1-8) | mG*fUmoe(MeC)*fC*moe(MeC)fU&mUmoeTfC*moeT*dC*d | | |
| | i&mUfC*moeG*mA*mU*mG&mG(GalNac) | | |
| AI-1209 | mG#mC*mCfC#fC*mA*fG*mCmoeAfG*fC*moeT#fU*mCfA*m | 199 | 11 |
| (25-1-8) | G*fUmoe(MeC)*fC*moe(MeC)fU#mUmoeTfC*moeT*dC*di#m | | |
| | UfC*moeG*mA*mU*mG#mG(GalNac) | | |

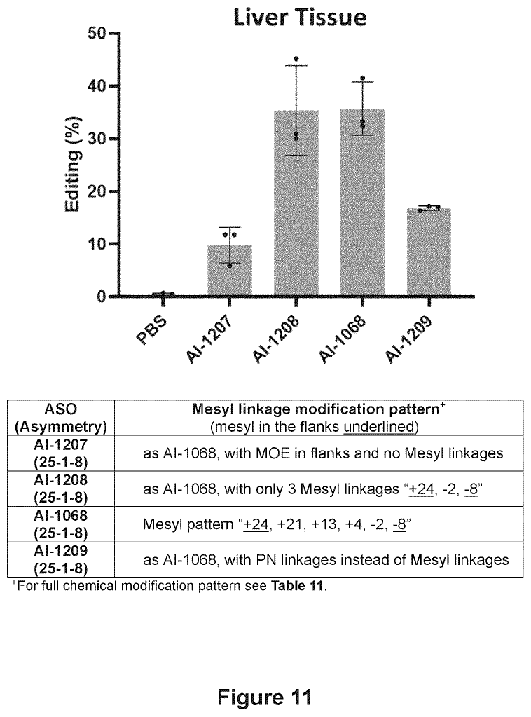

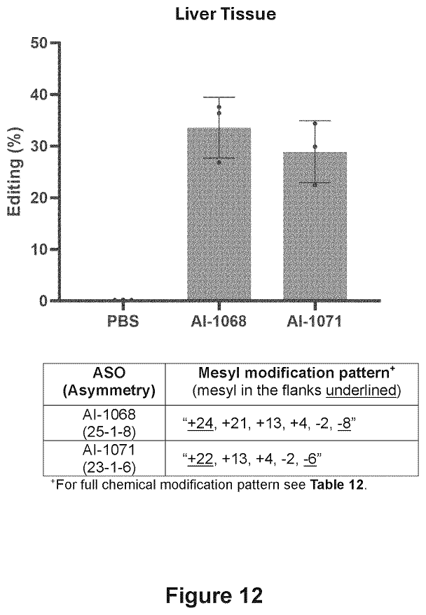

In AI-1207, replacement of all mesyl linkages and addition of 2′MOE modifications to the terminal 3 nucleotides of each the 5′ and 3′ termini resulted in a significant decrease in editing efficacy (FIG. 11). AI-1208 did not show a significant difference in editing efficacy when compared to AI-1068 (FIG. 11). Interestingly, in AI-1209, replacement of all mesyl linkage modifications by PN linkages at identical positions led to a significant decrease in editing, which was significantly lower than that of AI-1068 and AI-1208 (FIG. 11).

Overall, it was shown that having mesyl linkages at the 5′ (+24) and 3 (-8) flanking positions and internally at position-2 is beneficial to the editing of the oligonucleotide. Moreover, these results show that there is a limit of as to the number of 2′MOE modifications in the 5′ and 3′ flanking regions. Too many 2′MOE modifications in the 5′ and 3′ flanks seems to have a detrimental effect on the overall editing efficacy. Similarly. PN linkages seem to be inferior to mesyl linkage modifications when placed in an identical modification pattern. The results indicate that mesyl linkages at the outmost 5′ and 3′ position are most important.

### Example 12

### Mesyl Linkage Modifications at the 5′ and 3′ Termini Improve In Vivo Editing for Different Asymmetries

A variety of 5′ and 3′ terminal modifications can be incorporated into a potential ASO candidate. However, the effect of these modifications may depend upon the length and symmetry of the specific ASO. To determine whether the positioning of the mesyl linkages at the 5′ and 3′ flanking positions has a positive impact on the editing of other ASO asymmetries and can be applied to different ASO asymmetries, AI-1071 (“23-1-6” asymmetry) was compared to AI-1068 (“25-1-8” asymmetry) for its in vivo editing efficacy.

In vivo study: In vivo studies were performed as described under Example 7 above and ASOs used at a concentration 3×10 mg/kg.

AI-1071 is a 30 nt long, SERPINA 1-specific ASO and contains 11 2′F modifications and S mesyl linkage modifications at positions at the 5′ and 3′ terminal flanking positions and internal positions +13, +4, and-2 (mesyl pattern “+22, +13, +4, −2, and −6”). PBS was used as negative control. The oligonucleotide constructs, their respective modification pattern and sequence are listed in Table 12.

Overall, these results show that incorporating mesyl linkages at the 5′ and 3′ flanking regions is beneficial, and this modification pattern can be applied not only to ASOs with a 25-1-8 asymmetry but also to ASOs with other symmetries. e.g., the 23-1-6 asymmetry. This pattern can also be extended to other asymmetrical configurations, such as, 20-1-6, 29-1-8, 27-1-6, and 26-1-6.

TABLE 12
Oligonucleotide constructs and modifications. d = 2′-H(deoxyribose; DNA);
* = phosphorothioate (PS); m = 2′-OMe; & = (mesyl) methanesulfonyl; f = 2′-fluoro;
moe = 2′-O-Methoxyethyl (2′-MOE); i = inosine; MeC = 5′-methylcytidine; # = phosphoryl
guanidine (PN); GalNac = N-acetylgalactosamine.

| Construct | | Seq. | Fig. |
|---|---|---|---|
| (Asymmetry) | Modification Pattern and Sequence (5′-3′ direction) | ID | No. |
| AI-1068 | mG&mC*mCfC&fC*mA*fG*mCmoeAfG*fC*moeT&fU*mCfA* | 192 | 12 |
| (25-1-8) | mG*fUmoe(MeC)*fC*moe(MeC)fU&mUmoeTfC*moeT*dC*di | | |
| | &mUfC*moeG*mA*mU*mG&mG(GalNac) | | |
| AI-1071 | mC&mC*fC*mA*fG*mCmoeAfG*fC*moeT&fU*mCfA*mG*fU | 200 | 12 |
| (23-1-6) | moe(MeC)*fC*moe(MeC)fU&mUmoeTfc | | |
| | *moeT*dC*di&mUfC*moeG*mA&mU(GalNAc) | | |

### Example 13

### Mesyl Linkage Modification at Position +13 Enhances Target Editing

To investigate the different combinations of chemical modifications, mesyl linkage modifications and asymmetries on the efficiency and accuracy of target editing, various constructs were generated and tested in vivo.

In vivo studies: In vivo studies were performed as described under Example 7 above, ASOs were used at a concentration 3×5 mg/kg.

The oligonucleotide constructs, their asymmetries, respective modification pattern and sequences are listed in Table 13. All constructs were generated having a base level of mesyl linkage modifications in the 5′ and 3′ terminal ends and at one or more internal positions.

TABLE 13
Oligonucleotide constructs and modifications. d = 2′-H(deoxyribose; DNA);
* = phosphorothioate (PS); m = 2′-OMe; & = (mesyl) methanesulfonyl; f = 2′-fluoro;
moe = 2′-O-Methoxyethyl (2′-MOE); i = inosine; MeC = 5′-methylcytidine; # = phosphoryl
guanidine (PN); SbU = iso-Uridine; ln = locked nuceic acid; GalNac = N-acetylgalactosamine.

| Construct | | Seq. | Fig. |
|---|---|---|---|
| (Asymmetry) | Modification Pattern and Sequence (5′-3′ direction) | ID | No. |
| AI-1068 | mG&mC*mCfC&fC*mA*fG*mCmoeAfG*fC*moeT&fU*mCfA* | 192 | 13 |
| (25-1-8) | mG*fUmoe(MeC)*fC*moe(MeC)fU&mUmoeTfC*moeT*dC*di | | |
| | &mUfC*moeG*mA*mU*mG&mG(GalNac) | | |
| AI-1699 | lnA&fG*mCmoeA*fG*fC*moeT&fU*mC*moeA*mG*moeTmoe | 201 | 13 |
| (20-1-6) | (MeC)*fC*moe(Mec)*fU&mUmoeTfC*moeT*dC*di&mUfC*mo | | |
| | eG*lnA&mU(GalNac) | | |
| AI-1700 | lnA&lnG*mCmoeA*fG*fC*moeT&fU*mC*moeA*mG*moeTmo | 202 | 13 |
| (20-1-6) | e(MeC)*fC*moe(Mec)*fU&mUmoeTfC*moeT*dC*di&mUfC*m | | |
| | eG&lnA&lnT(GalNac) | | |
| AI-1701 | lnA&lnG*mCmoeAfG*fC*moeT&fU*mCfA*mG*fUmoe(MeC)*f | 203 | 13 |
| (20-1-6) | C*moe(Mec)fU&mUmoeTfC*moeT*dC*di&mUfC*moeG*lnA& | | |
| | lnT(GalNac) | | |
| AI-1685 | lnA&fG*mCmoeAfG*fC*moeT&fU*mCfA*mG*fUmoe(MeC)*f | 204 | 13 |
| (20-1-6) | C*moe(Mec)fU&mUmoeTfC*moeT*dC*di&mUfC*moeG*lnA | | |
| | &mU(GalNac) | | |
| AI-1687 | mG&mC*mCfC*fC*mA*fG*mCmoeAfG*fC*moeT*fUmCfAm | 205 | 13 |
| (25-1-8) | G*fUmoe(MeC)*fCmoe(MeC)fU*mUmoeTfC*moeT*dC*di& | | |
| | mUfC*moeG*mA*mU*mG&mG(GalNac) | | |
| AI-1689 | lnA&lnG&mCmoeAfG*fC*moeT&fU*mCfA*mG*fUmoe(MeC) | 206 | 13 |
| (20-1-8) | *fC*moe(Mec)fU&mUmoeTfC*moeT*dC*di&mUfC*moeG&l | | |
| | nA&lnT(GalNac) | | |
| AI-1690 | mG&mC&mCfC*fC*mA*fG*mCmoeAfG*fC*moeT*fUmCfAm | 207 | 13 |
| (25-1-8) | G*fUmoe(MeC)*fCmoe(MeC)fU*mUmoeTfC*moeT*dC*di& | | |
| | mUfC*moeG*mA*mU&mG&mG(GalNac) | | |
| AI-1691 | mG&mC&mCfC&fC*mA*fG*mCmoeAfG*fC*moeT&fU*mCfA | 208 | 13 |
| (25-1-8) | *mG*fUmoe(MeC)*fC*moe(MeC)fU&mUmoeTfC*moeT*dC* | | |
| | di&mUfC*moeG*mA*mU&mG&mG(GalNac) | | |
| AI-1902 | lnA&lnG&mCmoeAfG*fC*moeT&fUmCfAmG*fUmoe(MeC)*fC | 209 | 13 |
| (20-1-6) | moe(Mec)fU&mUmoeTfC*moeT*dC*di&mUfC*moeG*lnA | | |
| | mU(GalNac) | | |
| AI-1849 | mC&mC*fC*mA*fG*mCmoeAfG*fC*moeT&fU*mCfA*mG*fUm | 210 | 13 |
| (23-1-6) | oe(MeC)*fC*moe(MeC)fU*mUmoeTfc | | |
| | *moeT*dC*di&mUfC*moeG*mA&mU(GalNAc) | | |
| AI-1850 | ln(MeC)&mC*fC*mA*fG*mCmoeAfG*fC*moeT&fU*mCfA*m | 211 | 13 |
| (23-1-6) | G*fUmoe(MeC)*fC*moe(MeC)fU*mUmoeTfc | | |
| | *moeT*dC*di&mUfC*moeG*lnA&mU(GalNAc) | | |
| AI-1986 | mG&mC*mCmC&fC*mA*fG*mCmoeAfG*mC*moeT&fU*mCf | 212 | 13 |
| (25-1-8) | A*mG*fUmoe(MeC)*fC*moe(MeC)fU&mUmoeTfC*moeT*dC | | |
| | *di&mUfC*moeG*mA*mU*mG&mG(GalNac) | | |
| AI-1901 | mG&mC*mCfC*fC*mA*fG*mCmoeAfG*fC*moeT&fUmCfAmG | 213 | 13 |
| (25-1-8) | *fUmoe(MeC)*fC*moe(MeC)fU*mUmoeTfC*moeT*dC*di&mUf | | |
| | C*moeG*mA*mU*mG&mG(GalNac) | | |
| AI-1952 | mG&mC*mCmC*fC*mA*fG*mCmoeAfG*mC*moeT*fUmCfA | 214 | 13 |
| (25-1-8) | mG*fUmoe(MeC)*fCmoe(MeC)fU*mUmoeTfC*moeT*dC*d | | |
| | i&mUfC*moeG*mA*mU*mG&mG(GalNac) | | |
| AI-1953 | mG&mC*mCmC*fC*mA*fG*mCmoeAfG*mC*moeT*fUmCf | 215 | 13 |
| (25-1-8) | AmG*fUmoe(MeC)*fCmoe(MeC)fU*mUmoeTfC*moeT*dC*d | | |
| | i&mUfC*moeG*mA*mU*mG&mG&mG(GalNac) | | |
| AI-1954 | mG&mCmCfC*fC*mA*fG*mCmoeAfG*fC*moeT*fUmCfAmo | 216 | 13 |
| (25-1-8) | eG*fUmoe(MeC)*fCmoe(MeC)fU*mUmoeTfC*moeT*dC*di& | | |
| | mUfC*moeG*mAmU*mG&mG(GalNac) | | |
| AI-1701 | moeG&mC*moe(MeC)fC&fC*mA*fG*mCmoeAfG*fC*moe&f | 217 | 13 |
| (25-1-8) | U*mCmoeA*mG*moeTmoe(MeC)*fC*moe(MeC)fU&mUmoeT | | |
| | fC*moeT*dC*di&mUfc*moeG*mA*moeT*mG&mG(GalNac) | | |
| AI-3163 | mG&mUmoe(MeC)fA*fG*mC*fi*mCmoeAfG*fi*moe(MeC)*fU* | 218 | 13 |
| (25-1-8) | mU*fA*moeT*mGmoe(MeC)*fA*moe(MeC)fi*mGmoe(MeC)fC | | |
| | *moeT*dC*di&mimA*moeG*mAmG*mC&moeT*(GalNac) | | |
| AI-3166 | mG&mUmoe(MeC)fA*fG*mC*fi*mCmoeAfG*fi*moe(MeC)*fU | 219 | 13 |
| (25-1-8) | *mU*fA*moeT*mGmoe(MeC)*fA*moe(MeC)fi*mGmoe(MeC)f | | |
| | C*moeT*dC*di&mimA*moeG*mAmG*mC&moeT*(GalNac) | | |

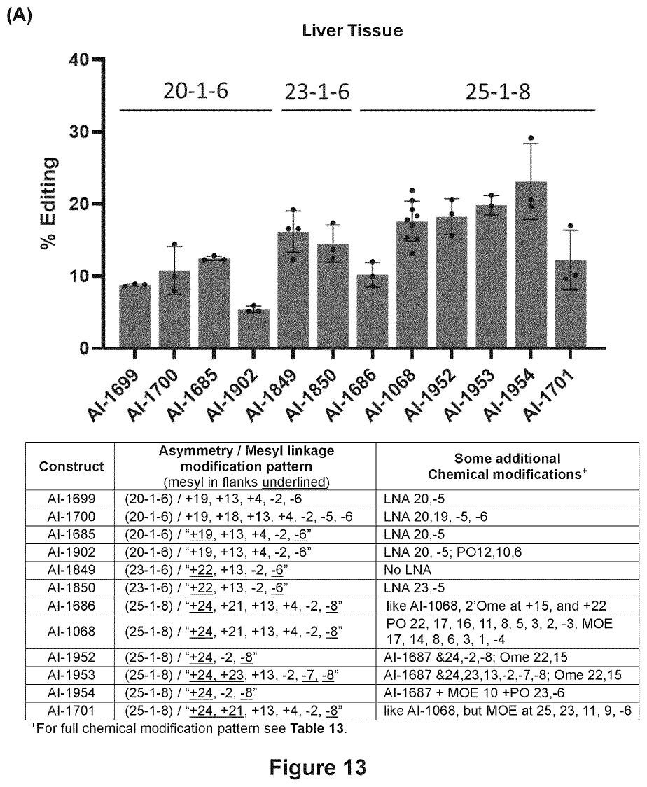

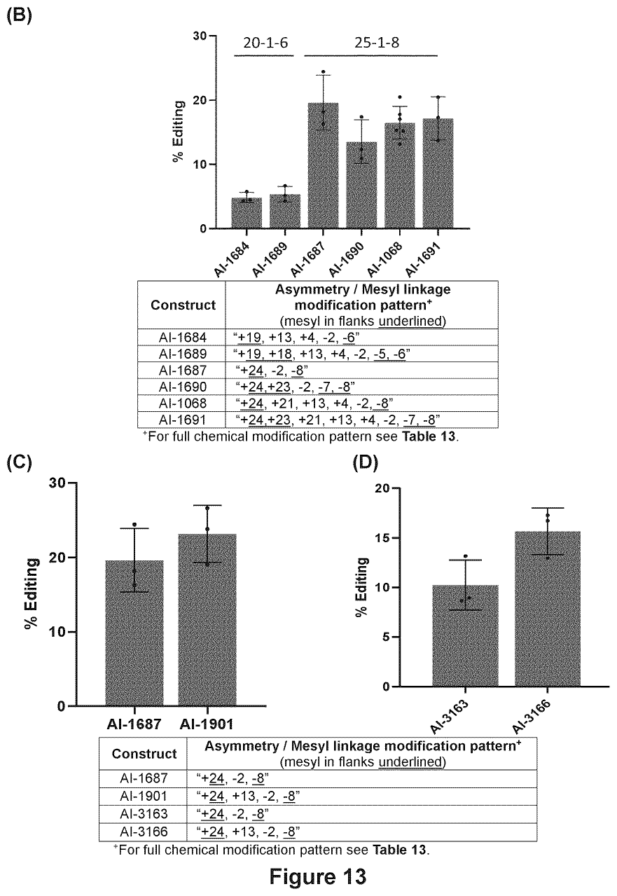

Regardless of their modification pattern, the shorter ASOs (“ 20-1-6 asymmetry”) showed the lowest level of target editing in vivo (FIG. 13(A). AI-1699, AI-1700, AI-1685, and AI-1902 all comprise locked nucleic acid (“In” or “LNA”) modification(s) at internal positions as well as at their 5′ and 3′ terminal ends. Notably, editing was lowest for AI-1902, which contains 2 “In” modifications and several mesyl linkage modifications (Table 13 and FIG. 13(A)). The results show that mesyl linkage modification in very short ASOs is not able to overcome the short length or asymmetry of the ASO and further demonstrates that ASO length and overall chemical modification pattern of the ASO cooperate to modulate target editing efficacy.

Slightly better editing was observed for ASOs having a “23-1-6” asymmetry AI-1850, which carries LNA modifications at the +23 and −6 position showed slightly lower editing when compared to AI-1849 (no LNA), suggesting that the presence of LNA modifications has a less negative impact on these oligonucleotides as compared to the 27 nt (“20-1-6”) long ASOs.

Good editing was also observed for AI-1068, which carries mesyl linkages at the outermost 5′ (+24) and 3′ (-8) flanking positions.

Generally, it was found that ASOs with a “25-1-8” asymmetry as well as shorter ASOs with a “23-1-6” asymmetry (AI-1849 and AI-1850) exhibited good target editing when mesyl linkage modifications were placed at the 5′ and 3′ terminal ends, i.e., at positions +24 and −8, and at positions +22 and 6, respectively. However, this was not the case for ASOs with a “20-1-6” asymmetry (FIGS. 13(A) and (B)). Also, mesyl linkages in the second outermost flank (penultimate position) was well tolerated for some oligonucleotide constructs and gave similar editing, e.g., AI-1691 vs AI-1068 and AI-1684 vs AI-1689. These data suggest that, while shorter ASOs still show good editing when having mesyl linkages at the outermost 5′ and 3′ flanking positions, ASOs that become too short showed little editing despite having mesyl linkages in their flanks. Moreover, it was shown that carrying too many 2″-MOE modifications decreases target editing regardless of having mesyl linkages in the 5′ and 3′ flanking regions (cf., AI-1068 vs AI-1701).

Further, it was shown that mesyl at position +13 seems to improve editing (FIG. 13(C). AI-1687 contains mesyl linkage modifications at the 5′ and 3′ flanking positions +24 and-8, and at the internal position-2 (“+24, −2, −8” pattern). AI-1901 corresponds to AI-1687 but contains an additional mesyl linkage at position +13. In vivo testing showed that having an additional mesyl linkage at position +13 has a positive effect on the editing efficacy of the ASO (FIG. 13(C). This effect was also observed for surrogate ASO constructs (FIG. 13(D)). Similarly, good editing was seen with AI-1953, which also contains a masyl linkage at position +13 and 2 mesyl linkages in each of the 5′ and 3′ flanks (FIG. 13(A)). These results suggest that a “24,-2,-8” mesyl linkage modification pattern is sufficient to increase editing in 34 nt ASOs and that an additional mesyl linkage modification at position +13 (see, AI-1901) seems to be beneficial for target editing.

Overall, these results show that mesyl linkage modifications can be important for efficient target editing. Specifically, mesyl linkage modifications in the 5′ and 3′ flanking regions seem to be beneficial to editing efficacy, that is, 1 or 2 mesyl linkages in each of the flanks is well tolerated and can improve target editing. The specific combination of ASO length and symmetry and mesyl linkages in the 5′ and 3′ flanking regions can lead to improved editing efficacy. Further, it was revealed that addition of too many 2′MOE modifications may have a negative impact on editing. Finally, it was shown that mesyl at position +13 can also improve editing.

### Example 14

### Oligonucleotide Surrogates Containing Mesyl Linkage Modifications Show Efficient In Vivo Target Editing

To further optimize the chemistry of ASO candidates and assess their potential to target other sites on the SERPINA1 target with improved stability and efficacy, ASO surrogate candidates were designed with a 25-1-8 asymmetry. These candidates featured a base mesyl-modification pattern of “+24, −2, −8” combined with additional PS, PO, and 2′-MOE modifications, variations in nucleotide sequences. PBS was used as control.

In vivo studies: In vivo studies were performed as described under Example 7 above. ASOs were used at a concentration 3×5 mg/kg.

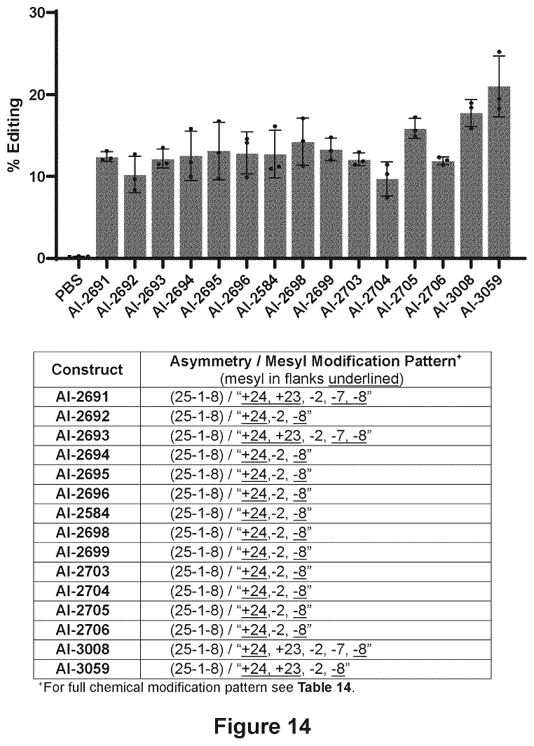

The oligonucleotide constructs, their respective modification pattern and sequence are listed in Table 14. The results are shown in FIG. 14.

TABLE 14
Surrogate olionucleotide constructs and modifications. d = 2′-H
(deoxyribose; DNA); * = phosphorothioate (PS); & = (mesyl) methanesulfonyl; m = 2′-OMe;
f = 2′-fluoro; moe = 2′-O-Methoxyethyl (2′-MOE); i = inosine; MeC = 5′-methylcytidine;
# = phosphoryl guanidine (PN); ln = locked nuceic acid.

| Construct | | Seq. | Fig. |
|---|---|---|---|
| (Asymmetry) | Modification Pattern and Sequence (5′-3′ direction) | ID | No. |
| AI-2691 | mG&mU&mCfA*fG*mC*fi*mC*moeAfG*fi*moe(MeC)fU*mUfA | 220 | 14 |
| (25-1-8) | *mU*fGmoe(MeC)*fAmoe(MeC)fi*mGmoe(MeC)fC*moeT*dC* | | |
| | *di&mifC*moeG*mAmG&mC&mU(GalNac) | | |
| AI-2692 | mG&mUmoe(MeC)fA*fG*mC*fi*mC*moeAfG*fi*moe(MeC)f | 221 | 14 |
| (25-1-8) | U*moeTfA*mU*mGmoe(MeC)*moeAmoe(MeC)fi*mGmoe(M | | |
| | eC)fC*moeT*dC*di&mi*fA*moeG*mAmG*mC&mU(GalNac) | | |
| AI-2693 | mG&mU&moe(MeC)fA*fG*mC*fi*mC*moeAfG*fi*moe(MeC) | 222 | 14 |
| (25-1-8) | fU*moeTfA*mU*fG*moe(MeC)*moeAmoe(MeC)fi*mGmoe | | |
| | (MeC)fC*moeT*dC*di&mi*fA*moeG*mAmG&mC&mU(GalNac) | | |
| AI-2694 | mG&mU&moe(MeC)fA*fG*mC*fi*mC*moeAfG*fi*moe(MeC)f | 223 | 14 |
| (25-1-8) | U*moeTfA*mU*fG*moe(MeC)*moeAmoe(MeC)fi*mGmoe(M | | |
| | eC)fC*moeT*dC*di&mi*fA*moeG*mAmG*mC&mU(GalNac) | | |
| AI-2695 | mG&mU&moe(MeC)fA*fG*mC*fi*mC*moeAfG*fi*moe(MeC)f | 224 | 14 |
| (25-1-8) | U*mUfA*mU*fGmoe(MeC)*moeAmoe(MeC)fi*mGmoe(MeC) | | |
| | fC*moeT*dC*di&mi*moeG*mAmG*fC&mU(GalNac) | | |
| AI-2696 | mG&mUmoe(MeC)fA*fG*mC*fi*mC*moeAfG*fi*moe(MeC)f | 225 | 14 |
| (25-1-8) | U*moeTfA*mUmGmoe(MeC)moeAmoe(MeC)fi*mGmoe(Me | | |
| | C)fC*moeT*dC*di&mi*fA*moeGmAmGmC&mU(GalNac) | | |
| AI-2584 | mG&mUmCfA*fG*mC*fi*mCmoeAfG*fi*moe(MeC)fU*mUfA* | 226 | 14 |
| (25-1-8) | mU*fGmoe(MeC)*fAmoe(MeC)fi*mGmoe(MeC)fC*moeT*dC* | | |
| | di&mifA*moeG*mA*mG*mC&mU(GalNac) | | |
| AI-2698 | mG&mUmoe(MeC)fA*fG*mC*fi*mC*moeAfG*fi*moe(MeC)f | 227 | 14 |
| (25-1-8) | U*moeTfA*mU*mGmoe(MeC)*moeAmoe(MeC)fi*mGmoe(M | | |
| | eC)fC*moeT*dC*di&mifA*moeG*mAmG*mC&mU(GalNac) | | |
| AI-2699 | mG&mUmCfA*fG*mC*fi*mC*moeAfG*fi*moe(MeC)fU*mUfA | 228 | 14 |
| (25-1-8) | *mU*fGmoe(MeC)*fAmoe(MeC)fi*mGmoe(MeC)fC*moeT*d | | |
| | C*di&mifA*moeG*mAmG*mC&mU(GalNac) | | |
| AI-2703 | mG&mUmCfA*fG*mCfi*mCmoeAfG*fi*moe(MeC)fU*mUfA* | 229 | 14 |
| (25-1-8) | mU*fGmoe(MeC)*fAmoe(MeC)fi*mGmoe(MeC)fC*moeT*dC | | |
| | *di&mifA*moeGmAmG*mC&mU(GalNac) | | |
| AI-2704 | mG&mUmCfA*fG*mCfA*mCmoeAfG*fC*moe(MeC)fU*mUfA | 230 | 14 |
| (25-1-8) | *mU*fGmoe(MeC)*fAmoe(MeC)fG*mGmoe(MeC)fC*moeT* | | |
| | dC*di&mifA*moeGmAmG*mC&mU(GalNac) | | |
| AI-2705 | mG&mUmCfA*fG*mC*fi*mCmoeAfG*fi*moe(MeC)*fU*mUfA | 231 | 14 |
| (25-1-8) | *mU*fGmoe(MeC)*fA*moe(MeC)fi*mGmoe(MeC)fC*moeT*d | | |
| | C*di&mifA*moeGmAmG*mC&mU(GalNac) | | |
| AI-2706 | mG&mUmCfA*fG*mC*fi*mC*moeAfG*fi*moe(MeC)*fU*mUf | 232 | 14 |
| (25-1-8) | A*mU*fGmoe(MeC)*fA*moe(MeC)fi*mGmoe(MeC)fC*moeT | | |
| | *dC*di&mifA*moeG*mAmG*mC&mU(GalNac) | | |
| AI-3008 | mG&mU&moe(MeC)fA*fG*mC*fi*mCmoeAfG*fi*moe(MeC)*f | 233 | 14 |
| (25-1-8) | U*moeTfA*mU*fG*moe(MeC)*moeAmoe(MeC)fi*mGmoe(Me | | |
| | C)fC*moeT*dC*di&mi*fA*moeG*mAmG&mC&mU(GalNac) | | |
| AI-3059 | mG&mU&moe(MeC)fA*fG*mC*fi*mCmoeAfG*fi*moe(MeC)*f | 234 | 14 |
| (25-1-8) | U*moeTfA*mU*fG*moe(MeC)*moeAmoe(MeC)fi*mGmoe(Me | | |
| | C)fC*moeT*dC*di&mifA*moeG*mAmG*mC&mU(GalNac) | | |

As shown in FIG. 14, all surrogate constructs showed good target editing. Surrogate constructs carrying 2 mesyl linkage modifications at each terminus showed good in vivo target editing. For instance, AI-2691 and AI-2693 (“+24, +23, −2, −7, −8”). Notably, even if only a single mesyl linkage modification was present in the 5′ and 2′ flanking regions (“+24, −2, −8”), good editing was observed. As shown, for example, for AI-2695 and AI-2699, good in vivo editing was observed when only one mesyl linkage was present between the penultimate and terminal nucleotide. Interestingly, surrogates AI-3008 and AI-3059 were identified as the most effective surrogate candidate, carrying either a “+24, +23, −2, −7, −8” and “+24, +23, −7, −8” mesyl modification pattern respectively. Notably, introducing further 2′-MOE modifications did not significantly impact overall target editing.

Overall, these results serve as a proof of concept to show that ASO candidates can be designed that comprise a general base mesyl modification pattern and that such ASOs provide high editing in vivo. The modification pattern of “+24, −2, −8” (i.e., mesyl at the outermost internucleoside linkages and at internal position −2) provided in the example demonstrated excellent editing capability. The constructs can be further optimised by adjusting the combination of PO, PS and 2′OME modifications to target the desired site of activity with improved ASO stability.

### Example 15

### Identification of Preferred Oligonucleotide Length and Asymmetry for Efficient Editing of Human Beta Actin (hACTB)

Mutations in this gene cause Baraitser-Winter syndrome 1, which is characterized by intellectual disability with a distinctive facial appearance in human patients. Recurrent mutations in the human beta actin (hACTB) gene have been associated to cases of diffuse large 8-cell lymphoma. To investigate the combined effect of ASO length and asymmetry, as well as internucleosidic linkage and 2′-sugar modification different oligonucleotides were generated and assayed for their in vitro RNA editing efficacy of the human beta actin (hACTB) target as described under Example 1 above for the SERPINA 1 target, NGS amplicon sequencing also took place as described above. The primers use in Example 17 are listed in Table 15.

TABLE 18
hACTB Primer Sequences.

| | | | SEQ ID |
|---|---|---|---|
| Target | Primer | Sequence | NO: |
| ACTB | Forward | TCGTCGGCAGCGTCAGATGTGTATAAGAGACA | 235 |
| | primer | GCTCTCTAAGGAGAATGGCCCAG | |
| ACTB | Reverse | GTCTCGTGGGCTCGGAGATGTGTATAAGAGA | 236 |
| | primer | CAGGGCACGAAGGCTCATCATTC | |
| ACTB | Sense | CCCAAGTCCACACAGGGGAGGTGATCGCATT | 237 |
| | Primer | GCTTTCGTGTAAATTATGTAATGCATT | |

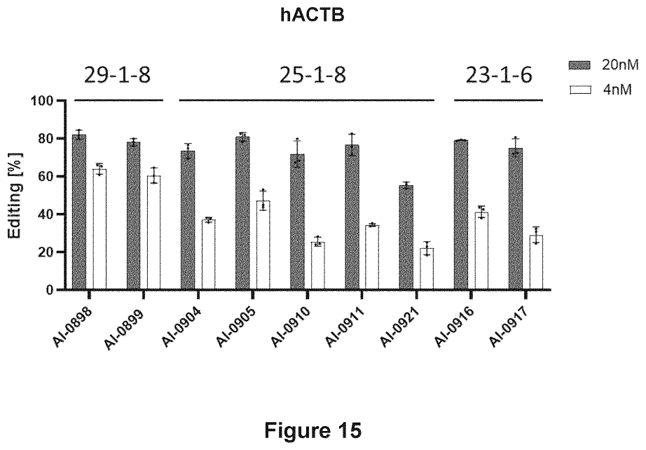

The different oligonucleotide constructs and their respective modifications used are listed in Table 16. The results are presented in FIG. 15.

TABLE 16
Oligonucleotide constructs and modifications.

| Construct | | |
|---|---|---|
| (nt) | Asymmetry, Description | FIG. No. |
| AI-0898 | 21F, 15 2′-OMe, 34 PS, PO 3, −2, −3 | 15 |
| (29−1−8) | | |
| AI-0899 | 21F, 15 2′-OMe, 32 PS, PO 3, −2, −3, Mesyl 28, −8 | 15 |
| (29−1−8) | | |
| AI-0904 | 18F, 14 2′-OMe, PO 3, −2, −3 | 16 |
| (25−1−8) | | |
| AI-0905 | 18F, 14 2′-OMe, PO 3, −2, −3, Mesyl 24, −8 | 15 |
| (25−1−8) | | |
| AI-0910 | 18F, 14 2′-OMe, PO 3, −3, PN −2 | 15 |
| (25−1−8) | | |
| AI-0911 | 18F, 14 2′-OMe, PO 3, −3, Mesyl 24, −8, PN −2 | 15 |
| (25−1−8) | | |
| AI-0921 | 12F, PO 22, 18, 16, 14, 11, 6, 3, 2, −3, PN −2, MOE 25, 24, 16, 14, | 16 |
| (25−1−8) | 8, 6, 1, −4, −6, −7, −8, Mesyl 24, 21, 13, 4, −8 | |
| AI-0916 | 17F, 11 ′2-OMe, PO 3, −2, −3 | 15 |
| (23−1−6) | | |
| AI-0917 | 17F 11 2′-OMe, PO 3, −2, −3 Mesyl 22, −6 | 15 |
| (23−1−6) | | |

PS = phosphorothioate; PO = phosphate; 2′-OMe = 2′-O-Methyl; F = 2′-fluoro; moe = 2′-O-Methoxyethyl (2′-MOE), I = inosine; MeC = 5′-methylcytidine; PN = phosphorly quanidine.

As shown in FIG. 15, there was a general decrease in target editing as the length of the oligonucleotide was decreased from 45 nt to 30 nt (see, AI-0111 (45 nt), AI-0235 (38 nt). AI-0243 (33 nt), and AI-0247 (30 nt)). Test ASOs such as AI-0927, which carries no mesyl linkage showed a dramatic reduction in editing. However, as seen with the SERPINA1 target, a combination of internucleoside linkage modification and 2′-sugar modifications was able to compensate for a decrease in oligonucleotide length. For instance, the shorter test construct AI-0905 [(34nt). 25-1-8, 18F, 14 2′-OMe, PO 3,-2,-3, Mesyl 24, −8] showed increased editing when compared to the longer AI-0235 construct and construct AI-0910 (34 nt), which lacks any mesyl linkages. Notably efficient editing was also obtained at lower concentrations of 4 nM.

Overall, these results show that the combination o particular internucleoside linkage modifications and 2′-sugar modifications as described herein can also be transferred to the hACTB target. Moreover, these data emphasize that having mesyl linkage modifications, particularly positioned in the outermost internucleoside positions, provides a beneficial effect on target editing.

### REFERENCES

1. Agrawal, S.; and Kandimalla, E. R. (2004). Antisense NA as agonists of Toll-like race Nature Biotechnol. 22: 1533-1537.
2. Sass, B.L.; Weintraub H. (1987). A developmentally regulated activity that unwinds RNA duplexes. Cell. 48: 607-13.
3. Bittner, Z. A.; Lu, X.; Shankar, S.; Tapia-Abellán, A.; Kalbacher, H.; Andreeva, L.; Mangan, M.; Düwell, P.; Lovotti, M.; Bosch, K.; Dickhöfer, S.; Marcu, A.; etc. (2021). BTK operates a phosphotyrosine switch to regulate NLRP3 inflammasome activity. JEM. 218(11): e20201656.
4. Cideciyan, A. V.; Jacobson, S. G.; Drack, A. V.; Ho, A. C.; Charng, J.; Garafalo, A. V.; et al. (2019) Effect of an intravitreal antisense oligonucleotide on vision in Leber congenital amaurosis due to a photoreceptor cilium defect. Nat. Med. 25(2): 225-228.
5. Crooke, S. T.; Vickers, T. A.; and Liang, X. (2020), Phosphorothicate modified oligonucleotide-protein interactions. Nucleic Acids Research. 48(10): 5235-5253.
6. Debacker, A. J.; Voutila, J.; Catley, M.; Blkey, D.; and Habib, N. (2020) Delivery of Oligonucleotides to the Liver with GalNAc: From Research to Registered Therapeutic Drug, Molecular Therapy. 28(8): 1789-1771.
7. Gabriel. C. M.; Pimentel. B. R.; Gomez, C. A.; Cedillo, I.; A. A. Rodriguez. (2022) Improved Purification of GalNAc-Conjugated Antisense Oligonucleotides Using Boronic Acids': Org. Process Res. Dev. 26: 413-421
8. Gagliardi, M., and Ashizawa, A. T. (2021). The Challenges and Strategies of Antisense Oligonucleotide Drug Delivery. Biomedicines. 9(4): 433.
9. Green and Sambrook, Molecular Cloning; A Laboratory Manual (4th ed., Cold Spring Harbor Laboratory Press, Cold Spring Harbor, N.Y. (2012).
10. Iwamoto, N.; Butler, D. C. O.; Svrzikapa, N.; Mohapatra, S.; Zlatev, I.; Sah, D. W. Y.; Meena, Standley, S. M. et al. (2017). Control of phosphorothioate stereochemistry substantially increases the efficacy of antisense oligonucleotides. Nat. Biotech. 35: 845-851.
11. Janas, M. M.; Jiang, Y; Schlegel, M. K.; Waldron, S.; Kuchimanchi, S.; and Barros, S. A., (2017). Impact of Oligonucleotide Structure, Chemistry, and Delivery Method on In Vitro Cytotoxicity, Nucleic Acid Therapeutics. 27(1):
12. Lorenz. P.; Baker, B. F.; Bennett, C. F.; and Spector, D. L. (1998). Phosphorothioate antisense oligonucleotides induce the formation of nuclear bodies. Mol. Biol, Cell. 9: 1007-1023.
13. Merkle, T.; Merz, S.; Reautschnig, P.; Blaha, A.; Li, Q., Vogel, P.; Wettengel, J; Li, J. B.; Stalforst, T. (2018). Precise RNA editing by recruiting endogenous ADARs with antisense oligonucleotides. Nature Biotechnol. 37: 133-138.
14. Miroshnichenko, S. K.; Patutina, O. A.; Burakova, E. A.; Cheiobanov, B. P.; Fokina, A. A.; Vlassov. V. V.; Allman. A.; Zenkova, M. A.; and Stetsenko, D. A. (2019). Mesyl phosphoramidate antisense oligonucleotides as an alternative to phosphorothioates with improved biochemical and biological properties. Proc. Natl. Acad. Sci. USA. 116(4): 1229-1234.
15. Patutina, O. A.; Gaponova (Miroshnichenko), S. K.; Sen'kova, A. V.; and Zenkova, M. A. (2020) Mesyl phosphoramidate backbone modified antisense oligonucleotides targeting miR-21 with enhanced in vivo therapeutic potency. PNAS. 117(51): 32370-32378.
16. Quemener, A. M.; Bachelot, L., Forestier, A.; Donnou-Fournet, E.; Gilot, D.; and Galibert, M.-D. (2019). The powerful world of antisense oligonucleotides: From bench to bedside. Wiley Interdiscip. Rev. RNA. 11(5): e1584.
17. Rebagliati, M.R.; Melton D. A. (1987). Antisense RNA injections in fertilized frog eggs reveal an RNA duplex unwinding activity. Cell 48:599-606. Zinshteyn, B., and Nishikura, K. (2009). Adenosine-to-Inosine RNA editing. Rev. Syst. Biol. Med. 1(2) 202-209.
18. Reynolds, M. A.; Hogrefe, R. I.; Jaeger. J. A.; Schwartz. D. A.; Riley, T. A.; et al. (1996). Synthesis and Thermodynamics of Oligonucleotides Containing Chirally Pure RP Methylphosphonate Linkages. Nucleic Acids Research: 24(22): 4584-4591.
19. Shen, W.; De Hoyos, C. L.; Migawa, M. T.; Vickers, T. A.; Sun, H.; Low, A.; Bell III, T. A.; Rahdar, M.; Mukhopadhyay, S.; Hart. C. E.; Bell, M.; Riney, G.; Murray, S. F.; Greenlee, G.; Crooke, R. M.; Liang, X.; Seth, S. S., and Crooke, S. T. (2019). Chemical modification of PS-ASO therapeutics reduces cellular protein-binding and improves the therapeutic Index. Nat. Biotech. 37:640-650.
20. Sothilingham, V.; Garrido, M. G.; Jiao, K.; Buena-Atienza, E.; Sahaboglu, A. et al. (2015), Retinitis pigmentosa: impact of different Pde6a point mutations on the disease phenotype. Human Mol. Genetics. 24(19): 5486-5499.
21. Stephenson, M. L; Zamecnik, P. C. (1978). Inhibition of Rous sarcoma viral RNA translation by a specific oligodeoxyribonucleotide, Proc. Natl. Acad. Sci. USA. 75(1): 285-288.
22. Thomas. J. M.; and Beal, P. A. (2017). How do ADARs bind RNA? New protein-RNA structures illuminate substrate recognition by the RNA editing ADARs, Bioassays, 38(4): 1-.
23. Tomaselli, S.; Locatelli, F.; Gallo, A. (2014). The RNA editing enzymes ADARs: mechanism of action and human disease. Cell and Tissue Research. 356:527-532.
24. Vogel, P.; Schneider, M. F.; Wettengel, J.; and Stafforst, T. (2014). Improving site-directed RNA editing in vitro and in cell culture by chemical modification of the guideRNA, Angew. Chem. Int. Ed. Engl. 53(24): 6267-6271.
25. Wooddell, C. I.; Blomenkamp, K.; Peterson, R. M.; Subbotin, V. M.; Schwabe, C.; Hamilton J.; etc. (2020). Development of an RNAi therapeutic for alpha-1-antitrypsin liver disease. JCI. 5(12): e135348.
26. Wulff, B. E.; and Nishikura, K. (2010). Substitutional A-to-I RNA editing. Interdiscip Rev. RNA. 1(1): 90-101.

1.-66. (canceled)

67. A chemically modified oligonucleotide comprising a nucleic acid sequence complementary to a target sequence in SERPINA1 messenger RNA (mRNA), wherein the chemically modified oligonucleotide comprises a central base triplet (CBT) of 3 nucleosides (5′-N₊₁N₀N₋₁-3′), wherein the central nucleoside of the CBT (N₀) is directly opposite to a target adenosine in the SERPINA1 mRNA that is to be edited to an inosine, wherein the target adenosine is the result of a G to A mutation in SERPINA1 gene that results in a E342K substitution in the alpha-1-antitrypsin (A1AT) protein,
wherein the chemically modified oligonucleotide is 34 nucleosides in length and has an asymmetry of 25-1-8 in a 5′ to 3′ direction, wherein the “1” indicates one nucleoside corresponding to N₀, positions of the 25 nucleosides 5′ of N₀ are numbered, from 5′ to 3′, +25 to +1, respectively, positions of the 8 nucleosides 3′ of N₀ are numbered, from 5′ to 3′, −1 to −8, respectively, positions of internucleoside linkages 5′ of N₀ are numbered, 5′ to 3′, +24 to 0, respectively, and positions of internucleoside linkages 5′ of N₀ are numbered, 5′ to 3′, −1 to −8, respectively, wherein each nucleoside of the chemically modified oligonucleotide is selected from: a 2′-O-Methyl (2′-O-Me) modified nucleoside, a 2′-fluoro (2′-F) modified nucleoside, a 2′-O-methoxy-ethyl (2′-MOE) modified nucleoside, and a 2′-deoxyribonucleoside (DNA),
wherein N₊₁ is a 2′-MOE modified nucleoside, N₀ is a DNA, and N₋₁ is a DNA; wherein the chemically modified oligonucleotide comprises a mismatch with respect to the target adenosine at position N₀;
wherein internucleoside linkages at positions +24, +13, −2, and −8 are methanesulfonyl (mesyl) linkages,
wherein at least 50% of the internucleoside linkages are phosphorothioate (PS) linkages, and wherein the PS linkages are not chirally controlled PS linkages.

68. The chemically modified oligonucleotide of claim 67, wherein the chemically modified oligonucleotide comprises at least one wobble base with respect to the target sequence.

69. The chemically modified oligonucleotide of claim 67, wherein at least 20% of nucleosides of the chemically modified oligonucleotide are 2′-F-modified nucleosides and at least 40% of nucleosides of the chemically modified oligonucleotide are 2′-O-Me-modified nucleosides.

70. The chemically modified oligonucleotide of claim 67, wherein 25%-55% nucleosides of the chemically modified oligonucleotide are 2′-O-Me modified nucleosides and 20%-30% nucleosides of the chemically modified oligonucleotide are 2′-F modified nucleosides.

71. The chemically modified oligonucleotide of claim 70, wherein the nucleosides at positions +22, +21, +19, +16, +15, +11, +7, +5, +2, and −3 are 2′-F modified nucleosides.

72. The chemically modified oligonucleotide of claim 71, wherein the nucleosides at positions 2′-MOE at positions +17, +14, +8, +6, +3, +1, and −4 are 2′-MOE modified nucleosides.

73. The chemically modified oligonucleotide of claim 72, comprising no more than 4 continuous 2′-O-Me modified nucleosides and/or no more than 4 continuous 2′-F modified nucleosides.

74. The chemically modified oligonucleotide of claim 73, wherein no more than 6% of nucleosides outside the CBT are DNAs.

75. The chemically modified oligonucleotide of claim 74, wherein internucleoside linkages at positions 1, 0, and −4 are PS linkages.

76. The chemically modified oligonucleotide of claim 75, wherein internucleoside linkages at positions +17, +16, +8, +5, +3, +2, and −3 are phosphate (PO) linkages.

77. The chemically modified oligonucleotide of claim 76, comprising the nucleobase sequence of SEQ ID NO: 192.

78. The chemically modified oligonucleotide of claim 76, wherein the chemically modified oligonucleotide does not comprise a loop-hairpin structured ADAR recruitment motif.

79. The chemically modified oligonucleotide of claim 78, further comprising a moiety that enhances cellular uptake of the chemically modified oligonucleotide.

80. The chemically modified oligonucleotide of claim 79, wherein the moiety that enhances cellular uptake is an N-acetyl galactosamine (GalNAc).

81. The chemically modified oligonucleotide of claim 80, wherein the GalNAc is conjugated to the 3′ terminus of the chemically modified oligonucleotide.

82. A composition comprising the oligonucleotide of claim 81, or a pharmaceutically acceptable salt thereof.

83. A method of inducing A-to-I editing of a target adenosine in a subject, comprising administering to the subject the chemically modified oligonucleotide of claim 81.

84. The method of claim 83, wherein the administration is subcutaneous.

85. The method of claim 84, wherein the subject is human.

86. The method of claim 85, wherein the subject has alpha-1-antitrypsin (A1AT) deficiency.


SEQUENCE LISTING (240 sequences)

SEQ ID NO: 1 - DNA, 45 bp, synthetic construct
  source 1..45: mol_type = other DNA
  misc_feature order(1..28,32..45): note = RNA
  misc_feature 29..31: note = DNA
  modified_base 31: mod_base = i
catggccccagcagcttcagtccctttctcntcgatggtcagcac

SEQ ID NO: 2 - DNA, 45 bp, synthetic construct; identical to SEQ ID NO: 1
  source 1..45: mol_type = other DNA
  misc_feature order(1..28,32..45): note = RNA
  misc_feature 29..31: note = DNA
  modified_base 31: mod_base = i

SEQ ID NO: 3 - DNA, 57 bp, synthetic construct
  source 1..57: mol_type = other DNA
tcgtcggcagcgtcagatgtgtataagagacaggcatcactaaggtcttcagcaatg

SEQ ID NO: 4 - DNA, 56 bp, synthetic construct
  source 1..56: mol_type = other DNA
gtctcgtgggctcggagatgtgtataagagacagggatagacatgggtatggcctc

SEQ ID NO: 5 - DNA, 37 bp, synthetic construct
  source 1..37: mol_type = other DNA
agctctccccggccgtgcataaggctgtgctgacata

SEQ ID NO: 6 - DNA, 30 bp, synthetic construct
  source 1..30: mol_type = other DNA
  misc_feature order(1..24,28..30): note = RNA
  misc_feature 25..27: note = DNA
  modified_base 27: mod_base = i
gccccagcagcttcagtccctttctcntcg

SEQ ID NO: 7 - DNA, 30 bp, synthetic construct
  source 1..30: mol_type = other DNA
  misc_feature order(1..23,27..30): note = RNA
  misc_feature 24..26: note = DNA
  modified_base 26: mod_base = i
ccccagcagcttcagtccctttctcntcga

SEQ ID NO: 8 - DNA, 30 bp, synthetic construct
  source 1..30: mol_type = other DNA
  misc_feature order(1..22,26..30): note = RNA
  misc_feature 23..25: note = DNA
  modified_base 25: mod_base = i
cccagcagcttcagtccctttctcntcgat

SEQ ID NO: 9 - DNA, 30 bp, synthetic construct
  source 1..30: mol_type = other DNA
  misc_feature order(1..21,24..30): note = RNA
  misc_feature 22..24: note = DNA
  modified_base 24: mod_base = i
ccagcagcttcagtccctttctcntcgatg

SEQ ID NO: 10 - DNA, 30 bp, synthetic construct
  source 1..30: mol_type = other DNA
  misc_feature order(1..20,24..30): note = RNA
  misc_feature 21..23: note = DNA
  modified_base 23: mod_base = i
cagcagcttcagtccctttctcntcgatgg

SEQ ID NO: 11 - DNA, 30 bp, synthetic construct
  source 1..30: mol_type = other DNA
  misc_feature order(1..19,23..30): note = RNA
  misc_feature 20..22: note = DNA
  modified_base 22: mod_base = i
agcagcttcagtccctttctcntcgatggt

SEQ ID NO: 12 - DNA, 32 bp, synthetic construct
  source 1..32: mol_type = other DNA
  misc_feature order(1..26,30..32): note = RNA
  misc_feature 27..29: note = DNA
  modified_base 29: mod_base = i
tggccccagcagcttcagtccctttctcntcg

SEQ ID NO: 13 - DNA, 32 bp, synthetic construct
  source 1..32: mol_type = other DNA
  misc_feature order(1..25,29..32): note = RNA
  misc_feature 26..28: note = DNA
  modified_base 28: mod_base = i
ggccccagcagcttcagtccctttctcntcga

SEQ ID NO: 14 - DNA, 32 bp, synthetic construct
  source 1..32: mol_type = other DNA
  misc_feature order(1..24,28..32): note = RNA
  misc_feature 25..27: note = DNA
  modified_base 27: mod_base = i
gccccagcagcttcagtccctttctcntcgat

SEQ ID NO: 15 - DNA, 32 bp, synthetic construct
  source 1..32: mol_type = other DNA
  misc_feature order(1..23,27..32): note = RNA
  misc_feature 24..26: note = DNA
  modified_base 26: mod_base = i
ccccagcagcttcagtccctttctcntcgatg

SEQ ID NO: 16 - DNA, 32 bp, synthetic construct
  source 1..32: mol_type = other DNA
  misc_feature order(1..22,26..32): note = RNA
  misc_feature 23..25: note = DNA
  modified_base 25: mod_base = i
cccagcagcttcagtccctttctcntcgatgg

SEQ ID NO: 17 - DNA, 32 bp, synthetic construct
  source 1..32: mol_type = other DNA
  misc_feature order(1..21,25..32): note = RNA
  misc_feature 22..24: note = DNA
  modified_base 24: mod_base = i
ccagcagcttcagtccctttctcntcgatggt

SEQ ID NO: 18 - DNA, 32 bp, synthetic construct
  source 1..32: mol_type = other DNA
  misc_feature order(1..20,24..32): note = RNA
  misc_feature 21..23: note = DNA
  modified_base 23: mod_base = i
cagcagcttcagtccctttctcntcgatggtc

SEQ ID NO: 19 - DNA, 32 bp, synthetic construct
  source 1..32: mol_type = other DNA
  misc_feature order(1..19,23..32): note = RNA
  misc_feature 20..22: note = DNA
  modified_base 22: mod_base = i
agcagcttcagtccctttctcntcgatggtca

SEQ ID NO: 20 - DNA, 34 bp, synthetic construct
  source 1..34: mol_type = other DNA
  misc_feature order(1..28,32..34): note = RNA
  misc_feature 29..31: note = DNA
  modified_base 31: mod_base = i
catggccccagcagcttcagtccctttctcntcg

SEQ ID NO: 21 - DNA, 34 bp, synthetic construct
  source 1..34: mol_type = other DNA
  misc_feature order(1..27,31..34): note = RNA
  misc_feature 28..30: note = DNA
  modified_base 30: mod_base = i
atggccccagcagcttcagtccctttctcntcga

SEQ ID NO: 22 - DNA, 34 bp, synthetic construct
  source 1..34: mol_type = other DNA
  misc_feature order(1..26,30..34): note = RNA
  misc_feature 27..29: note = DNA
  modified_base 29: mod_base = i
tggccccagcagcttcagtccctttctcntcgat

SEQ ID NO: 23 - DNA, 34 bp, synthetic construct
  source 1..34: mol_type = other DNA
  misc_feature order(1..25,29..34): note = RNA
  misc_feature 26..28: note = DNA
  modified_base 28: mod_base = i
ggccccagcagcttcagtccctttctcntcgatg

SEQ ID NO: 24 - DNA, 34 bp, synthetic construct
  source 1..34: mol_type = other DNA
  misc_feature order(1..24,28..34): note = RNA
  misc_feature 25..27: note = DNA
  modified_base 27: mod_base = i
gccccagcagcttcagtccctttctcntcgatgg

SEQ ID NO: 25 - DNA, 34 bp, synthetic construct
  source 1..34: mol_type = other DNA
  misc_feature order(1..23,27..34): note = RNA
  misc_feature 24..26: note = DNA
  modified_base 26: mod_base = i
ccccagcagcttcagtccctttctcntcgatggt

SEQ ID NO: 26 - DNA, 34 bp, synthetic construct
  source 1..34: mol_type = other DNA
  misc_feature order(1..22,26..34): note = RNA
  misc_feature 23..25: note = DNA
  modified_base 25: mod_base = i
cccagcagcttcagtccctttctcntcgatggtc

SEQ ID NO: 27 - DNA, 34 bp, synthetic construct
  source 1..34: mol_type = other DNA
  misc_feature order(1..21,25..34): note = RNA
  misc_feature 22..24: note = DNA
  modified_base 24: mod_base = i
ccagcagcttcagtccctttctcntcgatggtca

SEQ ID NO: 28 - DNA, 34 bp, synthetic construct
  source 1..34: mol_type = other DNA
  misc_feature order(1..20,24..34): note = RNA
  misc_feature 21..23: note = DNA
  modified_base 23: mod_base = i
cagcagcttcagtccctttctcntcgatggtcag

SEQ ID NO: 29 - DNA, 34 bp, synthetic construct
  source 1..34: mol_type = other DNA
  misc_feature order(1..19,23..34): note = RNA
  misc_feature 20..22: note = DNA
  modified_base 22: mod_base = i
agcagcttcagtccctttctcntcgatggtcagc

SEQ ID NO: 30 - DNA, 36 bp, synthetic construct
  source 1..36: mol_type = other DNA
  misc_feature order(1..28,32..36): note = RNA
  misc_feature 29..31: note = DNA
  modified_base 31: mod_base = i
catggccccagcagcttcagtccctttctcntcgat

SEQ ID NO: 31 - DNA, 36 bp, synthetic construct
  source 1..36: mol_type = other DNA
  misc_feature order(1..27,31..36): note = RNA
  misc_feature 28..30: note = DNA
  modified_base 30: mod_base = i
atggccccagcagcttcagtccctttctcntcgatg

SEQ ID NO: 32 - DNA, 36 bp, synthetic construct
  source 1..36: mol_type = other DNA
  misc_feature order(1..26,30..36): note = RNA
  misc_feature 27..29: note = DNA
  modified_base 29: mod_base = i
tggccccagcagcttcagtccctttctcntcgatgg

SEQ ID NO: 33 - DNA, 36 bp, synthetic construct
  source 1..36: mol_type = other DNA
  misc_feature order(1..25,29..36): note = RNA
  misc_feature 26..28: note = DNA
  modified_base 28: mod_base = i
ggccccagcagcttcagtccctttctcntcgatggt

SEQ ID NO: 34 - DNA, 36 bp, synthetic construct
  source 1..36: mol_type = other DNA
  misc_feature order(1..24,28..36): note = RNA
  misc_feature 25..27: note = DNA
  modified_base 27: mod_base = i
gccccagcagcttcagtccctttctcntcgatggtc

SEQ ID NO: 35 - DNA, 36 bp, synthetic construct
  source 1..36: mol_type = other DNA
  misc_feature order(1..23,27..36): note = RNA
  misc_feature 24..26: note = DNA
  modified_base 26: mod_base = i
ccccagcagcttcagtccctttctcntcgatggtca

SEQ ID NO: 36 - DNA, 36 bp, synthetic construct
  source 1..36: mol_type = other DNA
  misc_feature order(1..22,26..36): note = RNA
  misc_feature 23..25: note = DNA
  modified_base 25: mod_base = i
cccagcagcttcagtccctttctcntcgatggtcag

SEQ ID NO: 37 - DNA, 36 bp, synthetic construct
  source 1..36: mol_type = other DNA
  misc_feature order(1..21,25..36): note = RNA
  misc_feature 22..24: note = DNA
  modified_base 24: mod_base = i
ccagcagcttcagtccctttctcntcgatggtcagc

SEQ ID NO: 38 - DNA, 36 bp, synthetic construct
  source 1..36: mol_type = other DNA
  misc_feature order(1..20,24..36): note = RNA
  misc_feature 21..23: note = DNA
  modified_base 23: mod_base = i
cagcagcttcagtccctttctcntcgatggtcagca

SEQ ID NO: 39 - DNA, 36 bp, synthetic construct
  source 1..36: mol_type = other DNA
  misc_feature order(1..19,23..36): note = RNA
  misc_feature 20..22: note = DNA
  modified_base 22: mod_base = i
agcagcttcagtccctttctcntcgatggtcagcac

SEQ ID NO: 40 - DNA, 38 bp, synthetic construct
  source 1..38: mol_type = other DNA
  misc_feature order(1..28,32..38): note = RNA
  misc_feature 29..31: note = DNA
  modified_base 31: mod_base = i
catggccccagcagcttcagtccctttctcntcgatgg

SEQ ID NO: 41 - DNA, 38 bp, synthetic construct
  source 1..38: mol_type = other DNA
  misc_feature order(1..27,31..38): note = RNA
  misc_feature 28..30: note = DNA
  modified_base 30: mod_base = i
atggccccagcagcttcagtccctttctcntcgatggt

SEQ ID NO: 42 - DNA, 38 bp, synthetic construct
  source 1..38: mol_type = other DNA
  misc_feature order(1..26,30..38): note = RNA
  misc_feature 27..29: note = DNA
  modified_base 29: mod_base = i
tggccccagcagcttcagtccctttctcntcgatggtc

SEQ ID NO: 43 - DNA, 38 bp, synthetic construct
  source 1..38: mol_type = other DNA
  misc_feature order(1..25,29..38): note = RNA
  misc_feature 26..28: note = DNA
  modified_base 28: mod_base = i
ggccccagcagcttcagtccctttctcntcgatggtca

SEQ ID NO: 44 - DNA, 38 bp, synthetic construct
  source 1..38: mol_type = other DNA
  misc_feature order(1..24,28..38): note = RNA
  misc_feature 25..27: note = DNA
  modified_base 27: mod_base = i
gccccagcagcttcagtccctttctcntcgatggtcag

SEQ ID NO: 45 - DNA, 38 bp, synthetic construct
  source 1..38: mol_type = other DNA
  misc_feature order(1..23,27..38): note = RNA
  misc_feature 24..26: note = DNA
  modified_base 26: mod_base = i
ccccagcagcttcagtccctttctcntcgatggtcagc

SEQ ID NO: 46 - DNA, 38 bp, synthetic construct
  source 1..38: mol_type = other DNA
  misc_feature order(1..22,26..38): note = RNA
  misc_feature 23..25: note = DNA
  modified_base 25: mod_base = i
cccagcagcttcagtccctttctcntcgatggtcagca

SEQ ID NO: 47 - DNA, 38 bp, synthetic construct
  source 1..38: mol_type = other DNA
  misc_feature order(1..21,25..38): note = RNA
  misc_feature 22..24: note = DNA
  modified_base 24: mod_base = i
ccagcagcttcagtccctttctcntcgatggtcagcac

SEQ ID NO: 48 - DNA, 38 bp, synthetic construct
  source 1..38: mol_type = other DNA
  misc_feature order(1..20,24..38): note = RNA
  misc_feature 21..23: note = DNA
  modified_base 23: mod_base = i
cagcagcttcagtccctttctcntcgatggtcagcaca

SEQ ID NO: 49 - DNA, 38 bp, synthetic construct
  source 1..38: mol_type = other DNA
  misc_feature order(1..19,23..38): note = RNA
  misc_feature 20..22: note = DNA
  modified_base 22: mod_base = i
agcagcttcagtccctttctcntcgatggtcagcacag

SEQ ID NO: 50 - DNA, 40 bp, synthetic construct
  source 1..40: mol_type = other DNA
  misc_feature order(1..28,32..40): note = RNA
  misc_feature 29..31: note = DNA
  modified_base 31: mod_base = i
catggccccagcagcttcagtccctttctcntcgatggtc

SEQ ID NO: 51 - DNA, 40 bp, synthetic construct
  source 1..40: mol_type = other DNA
  misc_feature order(1..27,31..40): note = RNA
  misc_feature 28..30: note = DNA
  modified_base 30: mod_base = i
atggccccagcagcttcagtccctttctcntcgatggtca

SEQ ID NO: 52 - DNA, 40 bp, synthetic construct
  source 1..40: mol_type = other DNA
  misc_feature order(1..26,30..40): note = RNA
  misc_feature 27..29: note = DNA
  modified_base 29: mod_base = i
tggccccagcagcttcagtccctttctcntcgatggtcag

SEQ ID NO: 53 - DNA, 40 bp, synthetic construct
  source 1..40: mol_type = other DNA
  misc_feature order(1..25,29..40): note = RNA
  misc_feature 26..28: note = DNA
  modified_base 28: mod_base = i
ggccccagcagcttcagtccctttctcntcgatggtcagc

SEQ ID NO: 54 - DNA, 40 bp, synthetic construct
  source 1..40: mol_type = other DNA
  misc_feature order(1..24,28..40): note = RNA
  misc_feature 25..27: note = DNA
  modified_base 27: mod_base = i
gccccagcagcttcagtccctttctcntcgatggtcagca

SEQ ID NO: 55 - DNA, 40 bp, synthetic construct
  source 1..40: mol_type = other DNA
  misc_feature order(1..23,27..40): note = RNA
  misc_feature 24..26: note = DNA
  modified_base 26: mod_base = i
ccccagcagcttcagtccctttctcntcgatggtcagcac

SEQ ID NO: 56 - DNA, 40 bp, synthetic construct
  source 1..40: mol_type = other DNA
  misc_feature order(1..22,26..40): note = RNA
  misc_feature 23..25: note = DNA
  modified_base 25: mod_base = i
cccagcagcttcagtccctttctcntcgatggtcagcaca

SEQ ID NO: 57 - DNA, 40 bp, synthetic construct
  source 1..40: mol_type = other DNA
  misc_feature order(1..21,25..40): note = RNA
  misc_feature 22..24: note = DNA
  modified_base 24: mod_base = i
ccagcagcttcagtccctttctcntcgatggtcagcacag

SEQ ID NO: 58 - DNA, 40 bp, synthetic construct
  source 1..40: mol_type = other DNA
  misc_feature order(1..20,24..40): note = RNA
  misc_feature 21..23: note = DNA
  modified_base 23: mod_base = i
cagcagcttcagtccctttctcntcgatggtcagcacagc

SEQ ID NO: 59 - DNA, 40 bp, synthetic construct
  source 1..40: mol_type = other DNA
  misc_feature order(1..19,23..40): note = RNA
  misc_feature 20..22: note = DNA
  modified_base 22: mod_base = i
agcagcttcagtccctttctcntcgatggtcagcacagcc

SEQ ID NO: 60 - DNA, 45 bp, synthetic construct; identical to SEQ ID NO: 1
  source 1..45: mol_type = other DNA
  misc_feature order(1..28,32..45): note = RNA
  misc_feature 29..31: note = DNA
  modified_base 31: mod_base = i

SEQ ID NO: 61 - DNA, 30 bp, synthetic construct; identical to SEQ ID NO: 8
  source 1..30: mol_type = other DNA
  misc_feature order(1..23,26..30): note = RNA
  misc_feature 24..25: note = DNA
  modified_base 25: mod_base = i

SEQ ID NO: 62 - DNA, 30 bp, synthetic construct; identical to SEQ ID NO: 8
  source 1..30: mol_type = other DNA
  misc_feature order(1..23,26..30): note = RNA
  misc_feature 24..25: note = DNA
  modified_base 25: mod_base = i

SEQ ID NO: 63 - DNA, 34 bp, synthetic construct; identical to SEQ ID NO: 24
  source 1..34: mol_type = other DNA
  misc_feature order(1..25,28..34): note = RNA
  misc_feature 26..27: note = DNA
  modified_base 27: mod_base = i

SEQ ID NO: 64 - DNA, 34 bp, synthetic construct; identical to SEQ ID NO: 22
  source 1..34: mol_type = other DNA
  misc_feature order(1..27,30..34): note = RNA
  misc_feature 28..29: note = DNA
  modified_base 29: mod_base = i

SEQ ID NO: 65 - DNA, 38 bp, synthetic construct; identical to SEQ ID NO: 40
  source 1..38: mol_type = other DNA
  misc_feature order(1..29,32..38): note = RNA
  misc_feature 30..31: note = DNA
  modified_base 31: mod_base = i

SEQ ID NO: 66 - DNA, 36 bp, synthetic construct; identical to SEQ ID NO: 36
  source 1..36: mol_type = other DNA
  misc_feature order(1..23,26..36): note = RNA
  misc_feature 24..25: note = DNA
  modified_base 25: mod_base = i

SEQ ID NO: 67 - RNA, 30 nt, synthetic construct
  source 1..30: mol_type = other RNA
  misc_feature order(1..23,26..30): note = RNA
  misc_feature 24..25: note = DNA
  modified_base 25: mod_base = i
cccagcagcttcagtccctttctcntcgat

SEQ ID NO: 68 - DNA, 34 bp, synthetic construct; identical to SEQ ID NO: 24
  source 1..34: mol_type = other DNA
  misc_feature order(1..25,28..34): note = RNA
  misc_feature 26..27: note = DNA
  modified_base 27: mod_base = i

SEQ ID NO: 69 - DNA, 34 bp, synthetic construct; identical to SEQ ID NO: 22
  source 1..34: mol_type = other DNA
  misc_feature order(1..27,30..34): note = RNA
  misc_feature 28..29: note = DNA
  modified_base 29: mod_base = i

SEQ ID NO: 70 - DNA, 38 bp, synthetic construct; identical to SEQ ID NO: 40
  source 1..38: mol_type = other DNA
  misc_feature order(1..29,32..38): note = RNA
  misc_feature 30..31: note = DNA
  modified_base 31: mod_base = i

SEQ ID NO: 71 - DNA, 36 bp, synthetic construct; identical to SEQ ID NO: 36
  source 1..36: mol_type = other DNA
  misc_feature order(1..23,26..36): note = RNA
  misc_feature 24..25: note = DNA
  modified_base 25: mod_base = i

SEQ ID NO: 72 - DNA, 30 bp, synthetic construct; identical to SEQ ID NO: 8
  source 1..30: mol_type = other DNA
  misc_feature order(1..23,26..30): note = RNA
  misc_feature 24..25: note = DNA
  modified_base 25: mod_base = i

SEQ ID NO: 73 - DNA, 34 bp, synthetic construct; identical to SEQ ID NO: 24
  source 1..34: mol_type = other DNA
  misc_feature order(1..25,28..34): note = RNA
  misc_feature 26..27: note = DNA
  modified_base 27: mod_base = i

SEQ ID NO: 74 - DNA, 34 bp, synthetic construct; identical to SEQ ID NO: 22
  source 1..34: mol_type = other DNA
  misc_feature order(1..27,30..34): note = RNA
  misc_feature 28..29: note = DNA
  modified_base 29: mod_base = i

SEQ ID NO: 75 - DNA, 38 bp, synthetic construct; identical to SEQ ID NO: 40
  source 1..38: mol_type = other DNA
  misc_feature order(1..29,32..38): note = RNA
  misc_feature 30..31: note = DNA
  modified_base 31: mod_base = i

SEQ ID NO: 76 - DNA, 36 bp, synthetic construct; identical to SEQ ID NO: 36
  source 1..36: mol_type = other DNA
  misc_feature order(1..23,26..36): note = RNA
  misc_feature 24..25: note = DNA
  modified_base 25: mod_base = i

SEQ ID NO: 77 - DNA, 30 bp, synthetic construct; identical to SEQ ID NO: 8
  source 1..30: mol_type = other DNA
  misc_feature order(1..23,26..30): note = RNA
  misc_feature 24..25: note = DNA
  modified_base 25: mod_base = i

SEQ ID NO: 78 - DNA, 34 bp, synthetic construct; identical to SEQ ID NO: 24
  source 1..34: mol_type = other DNA
  misc_feature order(1..25,28..34): note = RNA
  misc_feature 26..27: note = DNA
  modified_base 27: mod_base = i

SEQ ID NO: 79 - DNA, 34 bp, synthetic construct; identical to SEQ ID NO: 22
  source 1..34: mol_type = other DNA
  misc_feature order(1..27,30..34): note = RNA
  misc_feature 28..29: note = DNA
  modified_base 29: mod_base = i

SEQ ID NO: 80 - DNA, 38 bp, synthetic construct; identical to SEQ ID NO: 40
  source 1..38: mol_type = other DNA
  misc_feature order(1..29,32..38): note = RNA
  misc_feature 30..31: note = DNA
  modified_base 31: mod_base = i

SEQ ID NO: 81 - DNA, 36 bp, synthetic construct; identical to SEQ ID NO: 36
  source 1..36: mol_type = other DNA
  misc_feature order(1..23,26..36): note = RNA
  misc_feature 24..25: note = DNA
  modified_base 25: mod_base = i

SEQ ID NO: 82 - DNA, 30 bp, synthetic construct; identical to SEQ ID NO: 8
  source 1..30: mol_type = other DNA
  misc_feature order(1..23,26..30): note = RNA
  misc_feature 24..25: note = DNA
  modified_base 25: mod_base = i

SEQ ID NO: 83 - DNA, 34 bp, synthetic construct; identical to SEQ ID NO: 24
  source 1..34: mol_type = other DNA
  misc_feature order(1..25,28..34): note = RNA
  misc_feature 26..27: note = DNA
  modified_base 27: mod_base = i

SEQ ID NO: 84 - DNA, 34 bp, synthetic construct; identical to SEQ ID NO: 22
  source 1..34: mol_type = other DNA
  misc_feature order(1..27,30..34): note = RNA
  misc_feature 28..29: note = DNA
  modified_base 29: mod_base = i

SEQ ID NO: 85 - DNA, 38 bp, synthetic construct; identical to SEQ ID NO: 40
  source 1..38: mol_type = other DNA
  misc_feature order(1..29,32..38): note = RNA
  misc_feature 30..31: note = DNA
  modified_base 31: mod_base = i

SEQ ID NO: 86 - DNA, 36 bp, synthetic construct; identical to SEQ ID NO: 36
  source 1..36: mol_type = other DNA
  misc_feature order(1..23,26..36): note = RNA
  misc_feature 24..25: note = DNA
  modified_base 25: mod_base = i

SEQ ID NO: 87 - DNA, 30 bp, synthetic construct; identical to SEQ ID NO: 8
  source 1..30: mol_type = other DNA
  misc_feature order(1..23,26..30): note = RNA
  misc_feature 24..25: note = DNA
  modified_base 25: mod_base = i

SEQ ID NO: 88 - DNA, 34 bp, synthetic construct; identical to SEQ ID NO: 24
  source 1..34: mol_type = other DNA
  misc_feature order(1..25,28..34): note = RNA
  misc_feature 26..27: note = DNA
  modified_base 27: mod_base = i

SEQ ID NO: 89 - DNA, 34 bp, synthetic construct; identical to SEQ ID NO: 22
  source 1..34: mol_type = other DNA
  misc_feature order(1..27,30..34): note = RNA
  misc_feature 28..29: note = DNA
  modified_base 29: mod_base = i

SEQ ID NO: 90 - DNA, 38 bp, synthetic construct; identical to SEQ ID NO: 40
  source 1..38: mol_type = other DNA
  misc_feature order(1..29,32..38): note = RNA
  misc_feature 30..31: note = DNA
  modified_base 31: mod_base = i

SEQ ID NO: 91 - DNA, 36 bp, synthetic construct; identical to SEQ ID NO: 36
  source 1..36: mol_type = other DNA
  misc_feature order(1..23,26..36): note = RNA
  misc_feature 24..25: note = DNA
  modified_base 25: mod_base = i

SEQ ID NO: 92 - DNA, 30 bp, synthetic construct; identical to SEQ ID NO: 8
  source 1..30: mol_type = other DNA
  modified_base order(1..2,23,28..30): mod_base = OTHER; note = 2 prime-O-methoxyethyl nucleoside
  modified_base 1..2: mod_base = OTHER; note = 5 prime methyl nucleoside
  misc_feature order(1..23,26..30): note = RNA
  misc_feature 24..25: note = DNA
  modified_base 25: mod_base = i

SEQ ID NO: 93 - DNA, 34 bp, synthetic construct; identical to SEQ ID NO: 24
  source 1..34: mol_type = other DNA
  modified_base order(1..3,25,30,32..34): mod_base = OTHER; note = 2 prime-O-methoxyethyl nucleoside
  modified_base 2..3: mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 94 - DNA, 34 bp, synthetic construct; identical to SEQ ID NO: 22
  source 1..34: mol_type = other DNA
  modified_base order(1..3,27,30,32..34): mod_base = OTHER; note = 2 prime-O-methoxyethyl nucleoside
  misc_feature order(1..27,30..34): note = RNA
  misc_feature 28..29: note = DNA
  modified_base 29: mod_base = i

SEQ ID NO: 95 - DNA, 38 bp, synthetic construct; identical to SEQ ID NO: 40
  source 1..38: mol_type = other DNA
  modified_base order(1..3,29,34,36..38): mod_base = OTHER; note = 2 prime-O-methoxyethyl nucleoside
  modified_base 1: mod_base = OTHER; note = 5 prime methyl nucleoside
  misc_feature order(1..29,32..38): note = RNA
  misc_feature 30..31: note = DNA
  modified_base 31: mod_base = i

SEQ ID NO: 96 - DNA, 36 bp, synthetic construct; identical to SEQ ID NO: 36
  source 1..36: mol_type = other DNA
  modified_base order(1..2,23,28,34..36): mod_base = OTHER; note = 2 prime-O-methoxyethyl nucleoside
  modified_base order(1..2,34): mod_base = OTHER; note = 5 prime methyl nucleoside
  misc_feature order(1..23,26..36): note = RNA
  misc_feature 24..25: note = DNA
  modified_base 25: mod_base = i

SEQ ID NO: 97 - DNA, 30 bp, synthetic construct; identical to SEQ ID NO: 8
  source 1..30: mol_type = other DNA
  misc_feature order(1..23,26..30): note = RNA
  misc_feature 24..25: note = DNA
  modified_base 25: mod_base = i

SEQ ID NO: 98 - DNA, 34 bp, synthetic construct; identical to SEQ ID NO: 24
  source 1..34: mol_type = other DNA
  misc_feature order(1..25,28..34): note = RNA
  misc_feature 26..27: note = DNA
  modified_base 27: mod_base = i

SEQ ID NO: 99 - DNA, 36 bp, synthetic construct; identical to SEQ ID NO: 36
  source 1..36: mol_type = other DNA
  misc_feature order(1..23,26..36): note = RNA
  misc_feature 24..25: note = DNA
  modified_base 25: mod_base = i

SEQ ID NO: 100 - DNA, 30 bp, synthetic construct; identical to SEQ ID NO: 8
  source 1..30: mol_type = other DNA
  misc_feature order(1..23,26..30): note = RNA
  misc_feature 24..25: note = DNA
  modified_base 25: mod_base = i

SEQ ID NO: 101 - DNA, 34 bp, synthetic construct; identical to SEQ ID NO: 24
  source 1..34: mol_type = other DNA
  misc_feature order(1..25,28..34): note = RNA
  misc_feature 26..27: note = DNA
  modified_base 27: mod_base = i

SEQ ID NO: 102 - DNA, 36 bp, synthetic construct; identical to SEQ ID NO: 36
  source 1..36: mol_type = other DNA
  misc_feature order(1..23,26..36): note = RNA
  misc_feature 24..25: note = DNA
  modified_base 25: mod_base = i

SEQ ID NO: 103 - DNA, 30 bp, synthetic construct; identical to SEQ ID NO: 8
  source 1..30: mol_type = other DNA
  misc_feature order(1..23,26..30): note = RNA
  misc_feature 24..25: note = DNA
  modified_base 25: mod_base = i

SEQ ID NO: 104 - DNA, 34 bp, synthetic construct; identical to SEQ ID NO: 24
  source 1..34: mol_type = other DNA
  misc_feature order(1..25,28..34): note = RNA
  misc_feature 26..27: note = DNA
  modified_base 27: mod_base = i

SEQ ID NO: 105 - DNA, 36 bp, synthetic construct; identical to SEQ ID NO: 36
  source 1..36: mol_type = other DNA
  misc_feature order(1..23,26..36): note = RNA
  misc_feature 24..25: note = DNA
  modified_base 25: mod_base = i

SEQ ID NO: 106 - DNA, 30 bp, synthetic construct; identical to SEQ ID NO: 8
  source 1..30: mol_type = other DNA
  misc_feature order(1..23,26..30): note = RNA
  misc_feature 24..25: note = DNA
  modified_base 25: mod_base = i

SEQ ID NO: 107 - DNA, 34 bp, synthetic construct; identical to SEQ ID NO: 24
  source 1..34: mol_type = other DNA
  misc_feature order(1..25,28..34): note = RNA
  misc_feature 26..27: note = DNA
  modified_base 27: mod_base = i

SEQ ID NO: 108 - DNA, 36 bp, synthetic construct; identical to SEQ ID NO: 36
  source 1..36: mol_type = other DNA
  misc_feature order(1..23,26..36): note = RNA
  misc_feature 24..25: note = DNA
  modified_base 25: mod_base = i

SEQ ID NO: 109 - DNA, 34 bp, synthetic construct; identical to SEQ ID NO: 24
  source 1..34: mol_type = other DNA
  misc_feature order(1..25,28..34): note = RNA
  misc_feature 26..27: note = DNA
  modified_base 27: mod_base = i

SEQ ID NO: 110 - DNA, 34 bp, synthetic construct; identical to SEQ ID NO: 24
  source 1..34: mol_type = other DNA
  misc_feature order(1..25,28..34): note = RNA
  misc_feature 26..27: note = DNA
  modified_base 27: mod_base = i

SEQ ID NO: 111 - DNA, 30 bp, synthetic construct; identical to SEQ ID NO: 8
  source 1..30: mol_type = other DNA
  misc_feature order(1..23,26..30): note = RNA
  misc_feature 24..25: note = DNA
  modified_base 25: mod_base = i

SEQ ID NO: 112 - DNA, 34 bp, synthetic construct; identical to SEQ ID NO: 24
  source 1..34: mol_type = other DNA
  misc_feature order(1..25,28..34): note = RNA
  misc_feature 26..27: note = DNA
  modified_base 27: mod_base = i

SEQ ID NO: 113 - DNA, 34 bp, synthetic construct; identical to SEQ ID NO: 24
  source 1..34: mol_type = other DNA
  misc_feature order(1..25,28..34): note = RNA
  misc_feature 26..27: note = DNA
  modified_base 27: mod_base = i

SEQ ID NO: 114 - DNA, 34 bp, synthetic construct; identical to SEQ ID NO: 24
  source 1..34: mol_type = other DNA
  misc_feature order(1..25,28..34): note = RNA
  misc_feature 26..27: note = DNA
  modified_base 27: mod_base = i

SEQ ID NO: 115 - DNA, 36 bp, synthetic construct; identical to SEQ ID NO: 36
  source 1..36: mol_type = other DNA
  misc_feature order(1..23,26..36): note = RNA
  misc_feature 24..25: note = DNA
  modified_base 25: mod_base = i

SEQ ID NO: 116 - DNA, 34 bp, synthetic construct; identical to SEQ ID NO: 24
  source 1..34: mol_type = other DNA
  misc_feature order(1..25,28..34): note = RNA
  misc_feature 26..27: note = DNA
  modified_base 27: mod_base = i

SEQ ID NO: 117 - DNA, 34 bp, synthetic construct; identical to SEQ ID NO: 24
  source 1..34: mol_type = other DNA
  misc_feature order(1..25,28..34): note = RNA
  misc_feature 26..27: note = DNA
  modified_base 27: mod_base = i

SEQ ID NO: 118 - DNA, 30 bp, synthetic construct; identical to SEQ ID NO: 8
  source 1..30: mol_type = other DNA
  modified_base order(1..2,23,28..30): mod_base = OTHER; note = 2 prime-O-methoxyethyl nucleoside
  modified_base 1..2: mod_base = OTHER; note = 5 prime methyl nucleoside
  misc_feature order(1..23,26..30): note = RNA
  misc_feature 24..25: note = DNA
  modified_base 25: mod_base = i

SEQ ID NO: 119 - DNA, 34 bp, synthetic construct; identical to SEQ ID NO: 24
  source 1..34: mol_type = other DNA
  modified_base order(1..3,25,30,32..34): mod_base = OTHER; note = 2 prime-O-methoxyethyl nucleoside
  modified_base 2..3: mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 120 - DNA, 36 bp, synthetic construct; identical to SEQ ID NO: 36
  source 1..36: mol_type = other DNA
  modified_base order(1..2,23,28,34..36): mod_base = OTHER; note = 2 prime-O-methoxyethyl nucleoside
  modified_base order(1..2,34): mod_base = OTHER; note = 5 prime methyl nucleoside
  misc_feature order(1..23,26..36): note = RNA
  misc_feature 24..25: note = DNA
  modified_base 25: mod_base = i

SEQ ID NO: 121 - DNA, 34 bp, synthetic construct; identical to SEQ ID NO: 24
  source 1..34: mol_type = other DNA
  modified_base order(1..3,10,12,18,20,25,30,32..34): mod_base = OTHER; note = 2 prime-O-methoxyethyl nucleoside
  modified_base order(2..3,18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 122 - DNA, 34 bp, synthetic construct; identical to SEQ ID NO: 24
  source 1..34: mol_type = other DNA
  modified_base order(1..3,10,12,18,20,25,30,32..34): mod_base = OTHER; note = 2 prime-O-methoxyethyl nucleoside
  modified_base order(1..2,18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 123 - DNA, 34 bp, synthetic construct; identical to SEQ ID NO: 24
  source 1..34: mol_type = other DNA
  modified_base order(1..3,10,12,18,20,25,30,32..34): mod_base = OTHER; note = 2 prime-O-methoxyethyl nucleoside
  modified_base order(2..3,18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 124 - DNA, 34 bp, synthetic construct; identical to SEQ ID NO: 24
  source 1..34: mol_type = other DNA
  modified_base order(1..3,10,12,18,20,25,30,32..34): mod_base = OTHER; note = 2 prime-O-methoxyethyl nucleoside
  modified_base order(2..3,18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 125 - DNA, 34 bp, synthetic construct; identical to SEQ ID NO: 24
  source 1..34: mol_type = other DNA
  modified_base order(1..3,10,12,18,20,25,30,32..34): mod_base = OTHER; note = 2 prime-O-methoxyethyl nucleoside
  modified_base order(2..3,18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 126 - DNA, 34 bp, synthetic construct; identical to SEQ ID NO: 24
  source 1..34: mol_type = other DNA
  modified_base order(1..3,10,12,18,20,25,30,32..34): mod_base = OTHER; note = 2 prime-O-methoxyethyl nucleoside
  modified_base order(2..3,18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 127 - DNA, 34 bp, synthetic construct; identical to SEQ ID NO: 24
  source 1..34: mol_type = other DNA
  modified_base order(10,12,18,20,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 128 - DNA, 34 bp, synthetic construct; identical to SEQ ID NO: 24
  source 1..34: mol_type = other DNA
  modified_base order(10,12,18,20,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 129 - DNA, 34 bp, synthetic construct; identical to SEQ ID NO: 24
  source 1..34: mol_type = other DNA
  modified_base order(10,18,20,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 130 - DNA, 34 bp, synthetic construct; identical to SEQ ID NO: 24
  source 1..34: mol_type = other DNA
  modified_base order(1..3,10,12,18,20,25,30,32..34): mod_base = OTHER; note = 2 prime-O-methoxyethyl nucleoside
  modified_base order(2..3,18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 131 - DNA, 34 bp, synthetic construct; identical to SEQ ID NO: 24
  source 1..34: mol_type = other DNA
  modified_base order(10,12,18,20,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 132 - DNA, 34 bp, synthetic construct; identical to SEQ ID NO: 24
  source 1..34: mol_type = other DNA
  modified_base order(8..9,18,23): mod_base = OTHER; note = 2 prime-O-methoxyethyl nucleoside
  modified_base order(8,18): mod_base = OTHER; note = 5 prime methyl nucleoside
  misc_feature order(1..24,28..34): note = RNA
  misc_feature 25..27: note = DNA
  modified_base 27: mod_base = i

SEQ ID NO: 133 - DNA, 34 bp, synthetic construct; identical to SEQ ID NO: 24
  source 1..34: mol_type = other DNA
  modified_base order(1..3,8..9,18,23,32..34): mod_base = OTHER; note = 2 prime-O-methoxyethyl nucleoside
  modified_base order(2..3,8,18): mod_base = OTHER; note = 5 prime methyl nucleoside
  misc_feature order(1..24,28..34): note = RNA
  misc_feature 25..27: note = DNA
  modified_base 27: mod_base = i

SEQ ID NO: 134 - DNA, 34 bp, synthetic construct; identical to SEQ ID NO: 24
  source 1..34: mol_type = other DNA
  modified_base order(1..3,11,18,20,30,32..34): mod_base = OTHER; note = 2 prime-O-methoxyethyl nucleoside
  modified_base order(2..3,11,18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 135 - DNA, 34 bp, synthetic construct; identical to SEQ ID NO: 24
  source 1..34: mol_type = other DNA
  modified_base order(1..3,11,18,20,30,32..34): mod_base = OTHER; note = 2 prime-O-methoxyethyl nucleoside
  modified_base order(2..3,11,18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 136 - DNA, 34 bp, synthetic construct; identical to SEQ ID NO: 24
  source 1..34: mol_type = other DNA
  modified_base order(1..3,11,18,20,30,32..34): mod_base = OTHER; note = 2 prime-O-methoxyethyl nucleoside
  modified_base order(2..3,11,18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 137 - DNA, 34 bp, synthetic construct; identical to SEQ ID NO: 24
  source 1..34: mol_type = other DNA
  modified_base order(1..3,11,18,20,30,32..34): mod_base = OTHER; note = 2 prime-O-methoxyethyl nucleoside
  modified_base order(2..3,11,18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 138 - DNA, 34 bp, synthetic construct; identical to SEQ ID NO: 24
  source 1..34: mol_type = other DNA
  modified_base order(1..3,10,11,18,20,25,30,32..34): mod_base = OTHER; note = 2 prime-O-methoxyethyl nucleoside
  modified_base order(2..3,18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 139 - DNA, 34 bp, synthetic construct; identical to SEQ ID NO: 24
  source 1..34: mol_type = other DNA
  modified_base order(1..3,10,12,18,20,25,30,32..34): mod_base = OTHER; note = 2 prime-O-methoxyethyl nucleoside
  modified_base order(2,18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 140 - DNA, 34 bp, synthetic construct; identical to SEQ ID NO: 24
  source 1..34: mol_type = other DNA
  modified_base order(1..3,10,12,18,20,25,30,32..34): mod_base = OTHER; note = 2 prime-O-methoxyethyl nucleoside
  modified_base order(2,18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 141 - DNA, 34 bp, synthetic construct; identical to SEQ ID NO: 24
  source 1..34: mol_type = other DNA
  modified_base order(1..4,6,8,10,12,14,16,18,20,24..25,30..34): mod_base = OTHER; note = 2 prime-O-methoxyethyl nucleoside
  modified_base order(2..4,8,14,18,20,24): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 142 - DNA, 34 bp, synthetic construct; identical to SEQ ID NO: 24
  source 1..34: mol_type = other DNA
  modified_base order(1..2,4,6,8,10,12,14,16,18,20,24..25,30,32..34): mod_base = OTHER; note = 2 prime-O-methoxyethyl nucleoside
  modified_base order(2,4,8,14,18,20,24): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 143 - DNA, 30 bp, synthetic construct; identical to SEQ ID NO: 8
  source 1..30: mol_type = other DNA
  modified_base order(6..7,16,21): mod_base = OTHER; note = 2 prime-O-methoxyethyl nucleoside
  modified_base order(6,16): mod_base = OTHER; note = 5 prime methyl nucleoside
  misc_feature order(1..22,26..30): note = RNA
  misc_feature 23..25: note = DNA
  modified_base 25: mod_base = i

SEQ ID NO: 144 - DNA, 34 bp, synthetic construct; identical to SEQ ID NO: 24
  source 1..34: mol_type = other DNA
  misc_feature order(1..25,28..34): note = RNA
  misc_feature 26..27: note = DNA
  misc_feature 1: note = linked to GalNAc
  modified_base 27: mod_base = i

SEQ ID NO: 145 - DNA, 34 bp, synthetic construct; identical to SEQ ID NO: 24
  source 1..34: mol_type = other DNA
  modified_base order(9,11,18,20,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 146 - DNA, 34 bp, synthetic construct; identical to SEQ ID NO: 24
  source 1..34: mol_type = other DNA
  modified_base order(9,12,18,20,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  misc_feature 1: note = linked to GalNAc
  modified_base 27: mod_base = i

SEQ ID NO: 147 - DNA, 34 bp, synthetic construct; identical to SEQ ID NO: 24
  source 1..34: mol_type = other DNA
  modified_base order(1..3,10,12,18,20,25,29..34): mod_base = OTHER; note = 2 prime-O-methoxyethyl nucleoside
  modified_base order(2..3,18,20): mod_base = OTHER; note = 5 prime methyl nucleoside
  misc_feature order(1..25,28..34): note = RNA
  misc_feature 26..27: note = DNA
  misc_feature 1: note = linked to GalNAc
  modified_base 27: mod_base = i

SEQ ID NO: 148 - DNA, 34 bp, synthetic construct; identical to SEQ ID NO: 24
  source 1..34: mol_type = other DNA
  misc_feature order(1..25,28..34): note = RNA
  misc_feature 26..27: note = DNA
  misc_feature 1: note = linked to GalNAc
  modified_base 27: mod_base = i

SEQ ID NO: 149 - DNA, 30 bp, synthetic construct; identical to SEQ ID NO: 8
  source 1..30: mol_type = other DNA
  misc_feature order(1..23,26..30): note = RNA
  misc_feature 24..25: note = DNA
  misc_feature 1: note = linked to GalNAc
  modified_base 25: mod_base = i

SEQ ID NO: 150 - DNA, 30 bp, synthetic construct; identical to SEQ ID NO: 8
  source 1..30: mol_type = other DNA
  modified_base order(6..7,16,21,23): mod_base = OTHER; note = 2 prime-O-methoxyethyl nucleoside
  modified_base order(6,16): mod_base = OTHER; note = 5 prime methyl nucleoside
  misc_feature order(1..22,26..30): note = RNA
  misc_feature 23..25: note = DNA
  misc_feature 1: note = linked to GalNAc
  modified_base 25: mod_base = i

SEQ ID NO: 151 - DNA, 30 bp, synthetic construct; identical to SEQ ID NO: 8
  source 1..30: mol_type = other DNA
  misc_feature order(1..23,26..30): note = RNA
  misc_feature 24..25: note = DNA
  misc_feature 1: note = linked to GalNAc
  modified_base 25: mod_base = i

SEQ ID NO: 152 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 153 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(28,30): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 154 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(28,30): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 155 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(28,30): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 156 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(28,30): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 157 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 158 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 159 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 160 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 161 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 162 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 163 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 164 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 165 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 166 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 167 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 168 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 169 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 170 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 171 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 172 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 173 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 174 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 175 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 176 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 177 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 178 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 179 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 180 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 181 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 182 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 183 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,27..34): note = RNA
  misc_feature 26: note = DNA
  modified_base 27: mod_base = i

SEQ ID NO: 184 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA

SEQ ID NO: 185 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 186 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,27..34): note = RNA
  misc_feature 26: note = DNA

SEQ ID NO: 187 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,27..34): note = RNA
  misc_feature 26: note = DNA
  modified_base 27: mod_base = i

SEQ ID NO: 188 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 189 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 190 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,27..34): note = RNA
  misc_feature 26: note = DNA
  modified_base 27: mod_base = i

SEQ ID NO: 191 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 192 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 193 - DNA, 34 bp, synthetic construct
  source 1..34: mol_type = other DNA
  modified_base order(9,12,23,30): mod_base = OTHER; note = 2 prime-O-methoxyethyl nucleoside
  modified_base order(18,20): mod_base = OTHER; note = 5 prime methyl nucleoside
  modified_base 26: mod_base = OTHER; note = isouridine
  misc_feature order(1..25,28..34): note = RNA
  misc_feature 26..27: note = DNA
  modified_base 27: mod_base = i
gccccagcagcttcagtccctttctnntcgatgg

SEQ ID NO: 194 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 195 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 196 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 197 - DNA, 34 bp, synthetic construct; identical to SEQ ID NO: 24
  source 1..34: mol_type = other DNA
  modified_base order(1,9,12,23,25,30,32..34): mod_base = OTHER; note = 2 prime-O-methoxyethyl nucleoside
  modified_base order(2..3,18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 198 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 199 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA

SEQ ID NO: 200 - DNA, 30 bp, synthetic construct; identical to SEQ ID NO: 8
  source 1..30: mol_type = other DNA
  modified_base order(7,10,21,22,28): mod_base = OTHER; note = 2 prime-O-methoxyethyl nucleoside
  modified_base order(16,18): mod_base = OTHER; note = 5 prime methyl nucleoside
  misc_feature order(1..23,26..30): note = RNA
  misc_feature 24..25: note = DNA
  modified_base 25: mod_base = i

SEQ ID NO: 201 - DNA, 27 bp, synthetic construct
  source 1..27: mol_type = other DNA
  modified_base order(4,7,10,12,18,20): mod_base = OTHER; note = 2 prime-O-methoxyethyl nucleoside
  modified_base order(13,15): mod_base = OTHER; note = 5 prime methyl nucleoside
  misc_feature order(1..20,23..27): note = RNA
  misc_feature 21..22: note = DNA
  modified_base 22: mod_base = i
agcagcttcagtccctttctcntcgat

SEQ ID NO: 202 - DNA, 27 bp, synthetic construct
  source 1..27: mol_type = other DNA
  modified_base order(4,7,10,12,18,20): mod_base = OTHER; note = 2 prime-O-methoxyethyl nucleoside
  modified_base order(13,15): mod_base = OTHER; note = 5 prime methyl nucleoside
  misc_feature order(1..20,23..27): note = RNA
  misc_feature 21..22: note = DNA
  modified_base 22: mod_base = i
agcagcttcagtccctttctcntcgat

SEQ ID NO: 203 - DNA, 27 bp, synthetic construct
  source 1..27: mol_type = other DNA
  modified_base order(4,7,10,12,18,20): mod_base = OTHER; note = 2 prime-O-methoxyethyl nucleoside
  modified_base order(13,15): mod_base = OTHER; note = 5 prime methyl nucleoside
  misc_feature order(1..20,23..27): note = RNA
  misc_feature 21..22: note = DNA
  modified_base 22: mod_base = i
agcagcttcagtccctttctcntcgat

SEQ ID NO: 204 - DNA, 27 bp, synthetic construct
  source 1..27: mol_type = other DNA
  modified_base order(4,7,10,12,18,20): mod_base = OTHER; note = 2 prime-O-methoxyethyl nucleoside
  modified_base order(13,15): mod_base = OTHER; note = 5 prime methyl nucleoside
  misc_feature order(1..20,23..27): note = RNA
  misc_feature 21..22: note = DNA
  modified_base 22: mod_base = i
agcagcttcagtccctttctcntcgat

SEQ ID NO: 205 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 206 - DNA, 27 bp, synthetic construct
  source 1..27: mol_type = other DNA
  modified_base order(4,7,18,20,25): mod_base = OTHER; note = 2 prime-O-methoxyethyl nucleoside
  modified_base order(13,15): mod_base = OTHER; note = 5 prime methyl nucleoside
  misc_feature order(1..20,23..27): note = RNA
  misc_feature 21..22: note = DNA
  modified_base 22: mod_base = i
agcagcttcagtccctttctcntcgat

SEQ ID NO: 207 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 208 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 209 - DNA, 27 bp, synthetic construct
  source 1..27: mol_type = other DNA
  modified_base order(4,7,18,20,25): mod_base = OTHER; note = 2 prime-O-methoxyethyl nucleoside
  modified_base order(23,25): mod_base = OTHER; note = 5 prime methyl nucleoside
  misc_feature order(1..20,23..27): note = RNA
  misc_feature 21..22: note = DNA
  modified_base 22: mod_base = i
agcagcttcagtccctttctcntcgat

SEQ ID NO: 210 - DNA, 30 bp, synthetic construct; identical to SEQ ID NO: 8
  source 1..30: mol_type = other DNA
  modified_base order(7,10,21,22,28): mod_base = OTHER; note = 2 prime-O-methoxyethyl nucleoside
  modified_base order(16,18): mod_base = OTHER; note = 5 prime methyl nucleoside
  misc_feature order(1..23,26..30): note = RNA
  misc_feature 24..25: note = DNA
  modified_base 25: mod_base = i

SEQ ID NO: 211 - DNA, 30 bp, synthetic construct; identical to SEQ ID NO: 8
  source 1..30: mol_type = other DNA
  modified_base order(7,10,21,23,28): mod_base = OTHER; note = 2 prime-O-methoxyethyl nucleoside
  modified_base 1: mod_base = OTHER; note = 5-methyl-cytosine
  misc_feature order(1..23,26..30): note = RNA
  misc_feature 24..25: note = DNA
  modified_base 25: mod_base = i

SEQ ID NO: 212 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 213 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 214 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 215 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 216 - DNA, 34 bp, synthetic construct; identical to SEQ ID NO: 24
  source 1..34: mol_type = other DNA
  modified_base order(9,12,23,25,30): mod_base = OTHER; note = 2 prime-O-methoxyethyl nucleoside
  modified_base order(18,20): mod_base = OTHER; note = 5 prime methyl nucleoside
  misc_feature order(1..25,28..34): note = RNA
  misc_feature 26..27: note = DNA

SEQ ID NO: 217 - DNA, 34 bp, synthetic construct; identical to SEQ ID NO: 24
  source 1..34: mol_type = other DNA
  modified_base order(1,9,12,15,17,23,25,30,32): mod_base = OTHER; note = 2 prime-O-methoxyethyl nucleoside
  modified_base order(3,12,18,20,23): mod_base = OTHER; note = 5 prime methyl nucleoside
  misc_feature order(1..25,28..34): note = RNA
  misc_feature 26..27: note = DNA
  modified_base 27: mod_base = i

SEQ ID NO: 218 - DNA, 34 bp, synthetic construct
  source 1..34: mol_type = other DNA
  modified_base order(9,16,25,30,34): mod_base = OTHER; note = 2 prime-O-methoxyethyl nucleoside
  modified_base order(3,12,18,20,23): mod_base = OTHER; note = 5 prime methyl nucleoside
  misc_feature order(1..25,28..34): note = RNA
  misc_feature 26..27: note = DNA
  modified_base order(7,11,21,27..28): mod_base = i
gtcagcncagncttatgcacngcctcnnagagct

SEQ ID NO: 219 - DNA, 34 bp, synthetic construct; identical to SEQ ID NO: 218
  source 1..34: mol_type = other DNA
  modified_base order(9,16,25,30,34): mod_base = OTHER; note = 2 prime-O-methoxyethyl nucleoside
  modified_base order(3,12,18,20,23): mod_base = OTHER; note = 5 prime methyl nucleoside
  misc_feature order(1..25,28..34): note = RNA
  misc_feature 26..27: note = DNA
  modified_base order(7,11,21,27..28): mod_base = i

SEQ ID NO: 220 - DNA, 34 bp, synthetic construct; identical to SEQ ID NO: 218
  source 1..34: mol_type = other DNA
  modified_base order(9,25,30): mod_base = OTHER; note = 2 prime-O-methoxyethyl nucleoside
  modified_base order(12,18,20,23): mod_base = OTHER; note = 5 prime methyl nucleoside
  misc_feature order(1..25,28..34): note = RNA
  misc_feature 26..27: note = DNA
  modified_base order(7,11,21,27..28): mod_base = i

SEQ ID NO: 221 - DNA, 34 bp, synthetic construct; identical to SEQ ID NO: 218
  source 1..34: mol_type = other DNA
  modified_base order(9,14,19,25,30): mod_base = OTHER; note = 2 prime-O-methoxyethyl nucleoside
  modified_base order(3,12,18,20,23): mod_base = OTHER; note = 5 prime methyl nucleoside
  misc_feature order(1..25,28..34): note = RNA
  misc_feature 26..27: note = DNA
  modified_base order(7,11,21,27..28): mod_base = i

SEQ ID NO: 222 - DNA, 34 bp, synthetic construct; identical to SEQ ID NO: 218
  source 1..34: mol_type = other DNA
  modified_base order(9,14,19,25,30): mod_base = OTHER; note = 2 prime-O-methoxyethyl nucleoside
  modified_base order(3,12,18,20,23): mod_base = OTHER; note = 5 prime methyl nucleoside
  misc_feature order(1..25,28..34): note = RNA
  misc_feature 26..27: note = DNA
  modified_base order(7,11,21,27..28): mod_base = i

SEQ ID NO: 223 - DNA, 34 bp, synthetic construct; identical to SEQ ID NO: 218
  source 1..34: mol_type = other DNA
  modified_base order(9,14,19,25,30): mod_base = OTHER; note = 2 prime-O-methoxyethyl nucleoside
  modified_base order(3,12,18,20,23): mod_base = OTHER; note = 5 prime methyl nucleoside
  misc_feature order(1..25,28..34): note = RNA
  misc_feature 26..27: note = DNA
  modified_base order(7,11,21,27..28): mod_base = i

SEQ ID NO: 224 - DNA, 34 bp, synthetic construct; identical to SEQ ID NO: 218
  source 1..34: mol_type = other DNA
  modified_base order(9,19,25,30): mod_base = OTHER; note = 2 prime-O-methoxyethyl nucleoside
  modified_base order(3,12,18,20,23): mod_base = OTHER; note = 5 prime methyl nucleoside
  misc_feature order(1..25,28..34): note = RNA
  misc_feature 26..27: note = DNA
  modified_base order(7,11,21,27..28): mod_base = i

SEQ ID NO: 225 - DNA, 34 bp, synthetic construct; identical to SEQ ID NO: 218
  source 1..34: mol_type = other DNA
  modified_base order(9,14,19,25,30): mod_base = OTHER; note = 2 prime-O-methoxyethyl nucleoside
  modified_base order(3,12,18,20,23): mod_base = OTHER; note = 5 prime methyl nucleoside
  misc_feature order(1..25,28..34): note = RNA
  misc_feature 26..27: note = DNA
  modified_base order(7,11,21,27..28): mod_base = i

SEQ ID NO: 226 - DNA, 34 bp, synthetic construct; identical to SEQ ID NO: 218
  source 1..34: mol_type = other DNA
  modified_base order(9,25,30): mod_base = OTHER; note = 2 prime-O-methoxyethyl nucleoside
  modified_base order(12,18,20,23): mod_base = OTHER; note = 5 prime methyl nucleoside
  misc_feature order(1..25,28..34): note = RNA
  misc_feature 26..27: note = DNA
  modified_base order(7,11,21,27..28): mod_base = i

SEQ ID NO: 227 - DNA, 34 bp, synthetic construct; identical to SEQ ID NO: 218
  source 1..34: mol_type = other DNA
  modified_base order(9,14,19,25,30): mod_base = OTHER; note = 2 prime-O-methoxyethyl nucleoside
  modified_base order(3,12,18,20,23): mod_base = OTHER; note = 5 prime methyl nucleoside
  misc_feature order(1..25,28..34): note = RNA
  misc_feature 26..27: note = DNA
  modified_base order(7,11,21,27..28): mod_base = i

SEQ ID NO: 228 - DNA, 34 bp, synthetic construct; identical to SEQ ID NO: 218
  source 1..34: mol_type = other DNA
  modified_base order(9,25,30): mod_base = OTHER; note = 2 prime-O-methoxyethyl nucleoside
  modified_base order(12,18,20,23): mod_base = OTHER; note = 5 prime methyl nucleoside
  misc_feature order(1..26,28..34): note = RNA
  misc_feature 27: note = DNA
  modified_base order(7,11,21,27..28): mod_base = i

SEQ ID NO: 229 - DNA, 34 bp, synthetic construct; identical to SEQ ID NO: 218
  source 1..34: mol_type = other DNA
  modified_base order(9,25,30): mod_base = OTHER; note = 2 prime-O-methoxyethyl nucleoside
  modified_base order(12,18,20,23): mod_base = OTHER; note = 5 prime methyl nucleoside
  misc_feature order(1..25,28..34): note = RNA
  misc_feature 26..27: note = DNA
  modified_base order(7,11,21,27..28): mod_base = i

SEQ ID NO: 230 - DNA, 34 bp, synthetic construct
  source 1..34: mol_type = other DNA
  modified_base order(9,25,30): mod_base = OTHER; note = 2 prime-O-methoxyethyl nucleoside
  modified_base order(12,18,20,23): mod_base = OTHER; note = 5 prime methyl nucleoside
  misc_feature order(1..25,28..34): note = RNA
  misc_feature 26..27: note = DNA
  modified_base 27..28: mod_base = i
gtcagcacagccttatgcacggcctcnnagagct

SEQ ID NO: 231 - DNA, 34 bp, synthetic construct; identical to SEQ ID NO: 218
  source 1..34: mol_type = other DNA
  modified_base order(9,25,30): mod_base = OTHER; note = 2 prime-O-methoxyethyl nucleoside
  modified_base order(12,18,20,23): mod_base = OTHER; note = 5 prime methyl nucleoside
  misc_feature order(1..25,28..34): note = RNA
  misc_feature 26..27: note = DNA
  modified_base order(7,11,21,27..28): mod_base = i

SEQ ID NO: 232 - DNA, 34 bp, synthetic construct; identical to SEQ ID NO: 218
  source 1..34: mol_type = other DNA
  modified_base order(9,25,30): mod_base = OTHER; note = 2 prime-O-methoxyethyl nucleoside
  modified_base order(12,18,20,23): mod_base = OTHER; note = 5 prime methyl nucleoside
  misc_feature order(1..25,28..34): note = RNA
  misc_feature 26..27: note = DNA
  modified_base order(7,11,21,27..28): mod_base = i

SEQ ID NO: 233 - DNA, 34 bp, synthetic construct; identical to SEQ ID NO: 218
  source 1..34: mol_type = other DNA
  modified_base order(9,14,19,25,30,32): mod_base = OTHER; note = 2 prime-O-methoxyethyl nucleoside
  modified_base order(3,22,18,20,23): mod_base = OTHER; note = 5 prime methyl nucleoside
  misc_feature order(1..25,28..34): note = RNA
  misc_feature 26..27: note = DNA
  modified_base order(7,11,21,27..28): mod_base = i

SEQ ID NO: 234 - DNA, 34 bp, synthetic construct; identical to SEQ ID NO: 218
  source 1..34: mol_type = other DNA
  modified_base order(9,14,19,25,30,32): mod_base = OTHER; note = 2 prime-O-methoxyethyl nucleoside
  modified_base order(3,12,18,20,23): mod_base = OTHER; note = 5 prime methyl nucleoside
  misc_feature order(1..25,28..34): note = RNA
  misc_feature 26..27: note = DNA
  modified_base order(7,11,21,27..28): mod_base = i

SEQ ID NO: 235 - DNA, 55 bp, synthetic construct
  source 1..55: mol_type = other DNA
tcgtcggcagcgtcagatgtgtataagagacagctctctaaggagaatggcccag

SEQ ID NO: 236 - DNA, 54 bp, synthetic construct
  source 1..54: mol_type = other DNA
gtctcgtgggctcggagatgtgtataagagacagggcacgaaggctcatcattc

SEQ ID NO: 237 - DNA, 58 bp, synthetic construct
  source 1..58: mol_type = other DNA
cccaagtccacacaggggaggtgatcgcattgctttcgtgtaaattatgtaatgcatt

SEQ ID NO: 238 - DNA, 53 bp, synthetic construct
  source 1..53: mol_type = other DNA
tcgtcggcagcgtcagatgtgtataagagacagcacccctgaagctctccaag

SEQ ID NO: 239 - DNA, 56 bp, synthetic construct; identical to SEQ ID NO: 4
  source 1..56: mol_type = other DNA

SEQ ID NO: 240 - DNA, 57 bp, synthetic construct
  source 1..57: mol_type = other DNA
gcataaggctgtgctgaccatcgacccgaaagggactgaagctgctggggccatgaa